(19) United States
(12) Patent Application Publication
(10) Pub. No.: US 2010/0298138 A1
(43) Pub. Date: Nov. 25, 2010

(54) Aminoazoline and Urea Derivatives for Combating Animal Pests

(75) Inventors: Christopher Koradin, Ludwigshafen (DE); Markus Kordes, Bobenheim-Roxheim (DE); Ernst Baumann, Dudenhofen (DE); Ronan Le Vezouet, Mannheim (DE); Deborah L. Culbertson, Fuquay Varina, NC (US)

Correspondence Address: BRINKS, HOFER, GILSON & LIONE, P.O. BOX 110285, RESEARCH TRIANGLE PARK, NC, 27709, US

(73) Assignee: BASF SE, Ludwigshafen (DE)

(21) Appl. No.: 12/600,852

(22) PCT Filed: May 23, 2008
(86) PCT No.: PCT/EP08/56377

§ 371 Date: Nov. 19, 2009

Related U.S. Application Data
(60) Provisional application No. 60/940,207, filed on May 25, 2007.

(51) Int. Cl. A01C 1/06; A01P 7/04; A01P 7/02; A01P 5/00; A01N 43/78; A61K 31/426; C07D 277/18; A61P 33/00; C07C 335/12; A61K 31/17; A01N 47/28; C07C 69/347; C07C 69/708; C07D 261/18; A61K 31/42; A01N 43/80; C07D 213/80; A61K 31/44; A01N 43/40; C07C 69/736; A61K 31/216; C07C 69/78; C07D 307/68; A61K 31/341; A01N 43/08

(52) U.S. Cl. 504/100; 514/370; 548/193; 564/28; 514/587; 560/180; 560/187; 548/248; 514/378; 546/322; 514/356; 560/61; 514/510; 546/298; 514/350; 560/107; 549/484; 514/471

(57) ABSTRACT

The present invention relates to the use of aminoazoline and urea derivatives for com-bating animal pests. The invention also relates to a method for controlling animal pests by using these compounds, to seed and to an agricultural and veterinary composition comprising said compounds and to specific azoline and urea derivatives.

The present invention relates to the use of aminoazoline and urea derivatives for combating animal pests. The invention also relates to a method for controlling animal pests by using these compounds, to seed and to an agricultural and veterinary composition comprising said compounds and to specific azoline and urea derivatives.

Animal pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating insects, arachnids and nematodes. It is therefore an object of the present invention to provide compounds having a good pesticidal activity and showing a broad activity spectrum against a large number of different animal pests, especially against difficult to control insects, arachnids and nematodes.

2-(Indan-2-ylamino)-oxazoline compounds and 2-(1,2,3,4-tetrahydronapht-2-ylamino)oxazoline, -thiazoline and -imidazoline compounds are described in EP-A-0251453 as having useful α₂-adrenergic properties and thus being useful in the treatment of hyper- or hypoglycaemia. A pesticidal activity of these compounds is not mentioned.

WO 2007/020377 describes the use of 2-(indan-1-ylamino)-thiazoline compounds and 2-(1,2,3,4-tetrahydronapht-1-ylamino)-thiazoline compounds for controlling insects, acarines, nematodes or molluscs.

It is therefore an object of the present invention to provide compounds that have a good pesticidal activity, in particular insecticidal activity, and show a broad activity spectrum against a large number of different animal pests, especially against difficult to control insects.

It has been found that these objectives can be achieved by compounds of the formula I below.

Therefore, in a first aspect, the invention relates to the use of compounds of formula (I) or of a salt thereof for combating animal pests

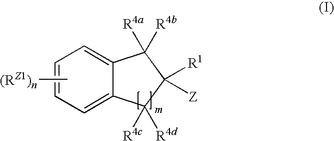

wherein
Z is a group of the formulae (II.A), (II.B) or (II.C)

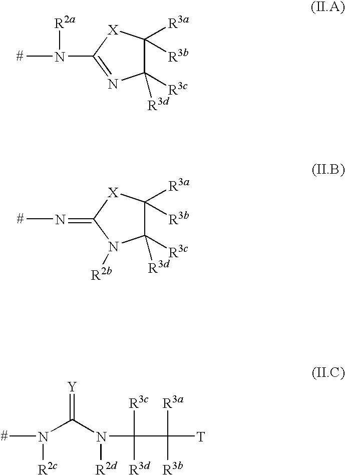

n is 0, 1, 2, 3 or 4;
m is 1 or 2;
# is the binding site to the remainder of the molecule;
X is S, O or NR⁵;
Y is S, O or NR^{5a};
T is —OR^{T1}, —SR^{T2}, —O—C(O)—R^{T3}, —O—C(S)—R^{T4} or —S—C(S)—R¹⁵;
R^{T1}, R^{T2}, R^{T3}, R^{T4} and R^{T5} are selected from hydrogen,
   C₁-C₆-alkyl, C₂-C₆-alkenyl or C₂-C₆-alkynyl, where the three last-mentioned radicals may carry 1, 2 or 3 substituents R^{Ta};
   C₃-C₆-cycloalkyl which may carry 1, 2 or 3 substituents R^{Tb};
   phenyl which may carry 1, 2 or 3 substituents R^{Tc}; where the phenyl ring may also be fused to another phenyl ring or to a 5- or 6-membered saturated, partially unsaturated or aromatic 5- or 6-membered heterocyclic ring containing 1, 2 or 3 heteroatoms selected from O, S and N and optionally 1 or 2 carbonyl groups as ring members, where the fused ring system may carry 1, 2 or 3 substituents R^{Tc}; saturated, partially unsaturated or aromatic 5- or 6-membered heterocyclyl containing 1, 2 or 3 heteroatoms selected from O, S and N and optionally 1 or 2 carbonyl groups as ring members, which may carry 1, 2 or 3 substituents R^{Td}; or
R^{T3}, R^{T4} and R^{T5} are NR^{T31}R^{T32}, where R^{T31} and R^{T32}, independently of each other, are selected from hydrogen, C₁-C₆-alkyl, C₂-C₆-alkenyl, C₂-C₆-alkynyl, where the 3 last-mentioned groups may carry 1, 2 or 3 substituents R^{Ta}, C₃-C₆-cycloalkyl, which may carry 1, 2 or 3 substituents R^{Tb}; aryl and aryl-C₁-C₄-alkyl, where the aryl moiety in the 2 last-mentioned groups may carry 1, 2 or 3 substituents R^{Tc}; or
   R^{T31} and R^{T32} together with the nitrogen atom to which they are bound form a 3-, 4-, 5- or 6-membered saturated, partially unsaturated or aromatic bered heterocyclic containing 1, 2 or 3 heteroatoms selected from O, S and N and optionally 1 or 2 carbonyl groups as ring members, where the heterocyclic ring may carry 1, 2 or 3 substituents R^{Td};
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
   C₃-C₆-cycloalkyl, phenyl or benzyl, phenoxycarbonyl, 5- or 6-membered hetaryl and 5- or 6-membered hetarylmethyl each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
   C(O)NR^{a}R^{b}, C(S)NR^{a}R^{b}, (SO₂)NR^{a}R^{b},
   phenyl, benzoyl, phenoxycarbonyl, benzyl, benzylcarbonyl, benzyloxycarbonyl, a 5- or 6-membered saturated, partially unsaturated or aromatic heterocyclic ring, 5- or 6-membered hetarylmethyl, 5- or 6-membered hetarylcarbonyl, 5- or 6-membered hetarylmethylcarbonyl, wherein each of the 10 last mentioned radicals may be unsubstituted or may carry any combination of 1, 2, 3, 4 or 5 radicals R^{b2}, and wherein the 5- or 6-membered heteroaromatic ring in hetarylmethyl, hetarylcarbonyl and hetarylmethylcarbonyl contains 1, 2, 3 or 4 heteroatoms selected from oxygen, sulfur and nitrogen as ring members, and wherein the 5- or 6-membered heterocyclic ring contains 1, 2 or 3 heteroatoms selected from oxygen, sulfur and nitrogen as ring members; or
R¹ together with R^{2a} may be C₃-C₅-alkandiyl which may carry 1, 2, 3, 4 or 5 radicals R²¹ and where one or two methylene groups of the C₃-C₅-alkandiyl radical may be replaced by 1 or 2 non-adjacent heteroatoms or heteroatom-containing groups selected from O, S and NR²²;
R¹ together with R^{2b} may also be a bridging carbonyl group C(O);
R^{2c} and R^{2d}, independently of each other, are selected from hydrogen, formyl, C₁-C₆-alkyl, C₂-C₆-alkenyl, C₂-C₆-alkynyl, aryl, aryl-C₁-C₄-alkyl, hetaryl, hetaryl-C₁-C₄-alkyl, C₁-C₆-alkylcarbonyl, C₁-C₆-alkylthiocarbonyl, C₂-C₆-alkenylcarbonyl, C₂-C₆-alkynylcarbonyl, C₁-C₆-alkoxycarbonyl, C₁-C₆-alkoxythiocarbonyl, arylcarbonyl, hetarylcarbonyl, aryloxycarbonyl and hetaryloxycarbonyl, where the aliphatic, aromatic or heteroaromatic moieties in the 17 last-mentioned radicals may carry 1, 2, 3, 4 or 5 radicals R^{c2}, and where hetaryl is a 5- or 6-membered heteroaromatic ring containing 1, 2, 3 or 4 heteroatoms selected from O, S and N as ring members;
R^{3a}, R^{3b}, R^{3c}, R^{3d}, independently of each other, are selected from the group consisting of hydrogen, halogen, cyano, nitro, hydroxy, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
   C₃-C₆-cycloalkyl, phenyl or benzyl, each of the last three mentioned radicals may be unsubstituted or may carry any combination of 1, 2, 3, 4 or 5 radicals R^{b3};
R^{4a}, R^{4b}, independently of each other, are selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
   phenyl, benzyl, 5 or 6 membered hetaryl and 5 or 6 membered hetarylmethyl, each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from oxygen, sulfur and nitrogen as ring members; or
R^{4a} together with R^{4b} may also be ═O, ═NR^{c} or ═CR^{d}R^{e}; or
R^{4a} together with R^{4b} may also be C₂-C₅-alkandiyl which may carry 1, 2, 3, 4, 5 or 6 substituents R⁴¹ and where one or two methylene groups of the C₂-C₅-alkandiyl radical may be replaced by 1 or 2 non-adjacent heteroatoms or heteroatom-containing groups selected from O, S and NR⁴²; or
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂O, S(O)—O, C(O)—NH, C(S)—NH, S(O)₂—NH, S(O)—NH;
R^{4c}, R^{4d} are independently defined like R^{4a} and R^{4b};
R⁵, R^{5a}, independently of each other, are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
   C(O)NR^{a}R^{b}, (SO₂)NR^{a}R^{b}, C(S)NR^{a}R^{b}
   phenyl, benzyl, phenoxycarbonyl, 5 or 6 membered hetarylmethyl, 5 or 6 membered hetarylcarbonyl and benzoyl each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from oxygen, sulfur and nitrogen as ring members;
each R^{Z1} is independently selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2 or 3 radicals R^{az},
   C(O)NR^{a}R^{b}, (SO₂)NR^{a}R^{b}, and radicals of the formula L-Cy, wherein
L is a single bond, oxygen, sulfur or C₁-C₆-alkandiyl, wherein one carbon might be replaced by oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from oxygen, sulfur and nitrogen as ring members, wherein Cy is unsubstituted or may carry any combination of 1, 2, 3, 4 or 5 radicals R^{bz};
   and wherein two radicals R^{Z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, or C₂-C₆-alkynyl, wherein the carbon atoms in these groups may carry any combination of 1, 2 or 3 radicals R^{aw};
R^{c} has one of the meanings given for R^{a} and R^{b} or is selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from C₁-C₆-alkoxy or di(C₁-C₆-alkyl)amino.
R^{a2}, R^{a3}, R^{a4}, R^{a5}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b1}, R^{b5}, R^{bc}, R^{bz} and R^{c2} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a}, R^{24b} and R⁴¹ have independently one of the meanings given for R^{b1} or two radicals R²¹ bound to the same carbon atom may together with this carbon atom form a carbonyl group or two radicals R⁴¹ bound to the same carbon atom may together with this carbon atom form a carbonyl group;
R²², R⁴² independently are hydrogen, C₁-C₆-alkyl, C₂-C₆-alkenyl, C₁-C₆-alkynyl, where the 3 last-mentioned groups may carry 1, 2 or 3 substituents R^{a42}, C₃-C₆-cycloalkyl, which may carry 1, 2 or 3 substituents R^{b42}; aryl and aryl-C₁-C₄-alkyl, where aryl moiety in the 2 last-mentioned groups may carry 1, 2 or 3 substituents R^{c42};
each R^{Ta} is independently selected from the group consisting of halogen, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy;
each R^{Tb} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₃-C₆-cycloalkoxy, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy;
each R^{Tc} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy;
each R^{Td} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy;
R^{a42} is independently defined like R^{Ta};
R^{b42} is independently defined like R^{Tb}; and
R^{c42} is independently defined like R^{Tc}.

Another object of the present invention is an agricultural composition containing at least one compound of the formula (I) as defined above and/or an agriculturally acceptable salt thereof and at least one liquid or solid carrier.

Another object of the present invention is a veterinary composition containing at least one compound of the formula (I) as defined above and/or a veterinarily acceptable salt thereof and at least one veterinarily acceptable liquid or solid carrier.

The present invention also provides a method for controlling animal pests which method comprises treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of a compound of the formula I or a salt thereof or a composition as defined above.

The invention further related to a method for treating or protecting an animal from infestation or infection by parasites which comprises bringing the animal in contact with a parasiticidally effective amount of a compound of the formula (I) or a veterinally acceptable salt thereof as defined above. Bringing the animal in contact with the compound (I), its salt or the veterinary composition of the invention means applying or administering it to the animal.

A further object of the present invention is seed, comprising at least one compound of formula (I) and/or an agriculturally acceptable salt thereof.

Object of the invention are furthermore azoline compounds of the general formula I as defined above and salts thereof, except for compounds wherein n is 0, or n is 1 or 2 with R^{Z1} being C₁-C₆-alkyl, C₁-C₆-alkoxy or halogen, Z is a group of formula (II.A) or (II.B) and R^{2a}, R^{2b}, R^{4a}, R^{4b}, R^{4c} and R^{4d} are all hydrogen.

Object of the invention are also azoline compounds of the general formula I as defined above and salts thereof, except for compounds wherein n is 0 or n is 1 or 2 with R^{Z1} being C₁-C₆-alkyl, C₁-C₆-alkoxy, halogen, phenyl or naphthyl, and simultaneously Z is a group of formula (II.A) or (II.B), R^{2a}, R^{2b}, R^{3a}, R^{3b}, R^{3c} and R^{3d} are all hydrogen and one of R^{4a} and R^{4b} is hydrogen and the other is hydrogen or C₁-C₆-alkyl and one of R^{4c} and R^{4d} is hydrogen and the other is hydrogen or C₁-C₆-alkyl.

In one embodiment, the present invention relates to azoline compounds of the general formula I as defined above and salts thereof, except compounds wherein R^{2a} and R^{2b} are both hydrogen.

In another embodiment the present invention also relates to azoline compounds of the general formula I as defined above and salts thereof, except compounds wherein R^{4a}, R^{4b}, R^{4c} and R^{4d} are all hydrogen.

In yet another embodiment the present invention relates to azoline compounds of the general formula I as defined above and salts thereof, except compounds wherein n is 0 or n is 1 or 2 and R^{Z1} is C₁-C₆-alkyl, C₁-C₆-alkoxy or halogen.

In the compounds of formula (I), the carbon atom which carries the radical R¹ creates a center of chirality. If there is no mirror plane σ through this atom, which is for example the case when m is 2 and/or at least one of R^{4a}, R^{4b}, R^{4c} and R^{4d} is not hydrogen, the saturated cycle however not being symmetrically substituted, and/or n is not 0, the phenyl ring however not being symmetrically substituted, the compound (I) can be present in the form of different enantiomers or diastereomers, depending on the substituents. In case Z is of the formula (II.B), the compound (I) may also exist as a cis- or trans-isomer with respect to the N═C axis. The present invention relates to every possible stereoisomer of the compounds of general formula (I), i.e. to single enantiomers or diastereomers, as well as to mixtures thereof.

Salts of the compounds of the formula (I) are preferably agriculturally and veterinarily acceptable salts. They can be formed in a customary method, e.g. by reacting the compound with an acid of the anion in question if the compound of formula I has a basic functionality or by reacting an acidic compound of formula (I) with a suitable base.

Suitable agriculturally useful salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH₄⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyltriethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula ( )I containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

Examples of other meanings are:

The term “C₁-C₆-alkyl” as used herein and in the alkyl moieties of C₁-C₆-alkoxy, C₁-C₆-alkylamino, di(C₁-C₆-alkyl)amino, C₁-C₆-alkylthio, C₁-C₆-alkylsulfonyl, C₁-C₆-alkylsulfoxyl, C₁-C₆-alkylcarbonyl, C₁-C₆-alkoxycarbonyl, C₁-C₆-alkylthiocarbonyl, C₁-C₆-alkoxythiocarbonyl, and C₁-C₆-alkylcarbonyloxy refer to a saturated straight-chain or branched hydrocarbon group having 1 to 6 carbon atoms, especially 1 to 4 carbon groups (═C₁-C₄-alkyl). Examples for C₁-C₄-alkyl are methyl, ethyl, propyl, 1-methylethyl (isopropyl), butyl, 1-methylpropyl (sec-butyl, 2-butyl), 2-methylpropyl (isobutyl), and 1,1-dimethylethyl (tert-butyl). Examples for C₁-C₆-alkyl further encompass pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl.

The term “C₁-C₆-haloalkyl” as used herein refers to a straight-chain or branched saturated alkyl group having 1 to 6 carbon atoms (as mentioned above), where some or all of the hydrogen atoms in these groups may be replaced by halogen atoms as mentioned above, for example C₁-C₄-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and the like.

The term “C₁-C₆-alkoxy” as used herein refers to a straight-chain or branched saturated alkyl group having 1 to 6 carbon atoms, preferably 1 to 4 carbon atoms (═C₁-C₄-alkoxy) (as mentioned above) which is attached via an oxygen atom. Examples for C₁-C₄-alkoxy include methoxy, ethoxy, OCH₂—C₂H₅ (propoxy), OCH(CH₃)₂ (isopropoxy), n-butoxy, OCH(CH₃)C₂H₅ (sec-butoxy), OCH₂CH(CH₃)₂ (isobutoxy) and OC(CH₃)₃ (tertbutoxy). Examples for C₁-C₆-alkoxy further encompass n-pentoxy, 1-methylbutoxy, 2-methylbutoxy, 3-methylbutoxy, 1,1-dimethylpropoxy, 1,2-dimethylpropoxy, 2,2-dimethyl-propoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, 1-ethyl-2-methylpropoxy and the like.

The term “C₁-C₆-haloalkoxy” as used herein refers to a C₁-C₆-alkoxy group as mentioned above wherein the hydrogen atoms are partially or fully substituted by fluorine, chlorine, bromine and/or iodine. Preferred are C₁-C₄-haloalkoxy groups, i.e. C₁-C₄-alkoxy groups as mentioned above wherein the hydrogen atoms are partially or fully substituted by fluorine, chlorine, bromine and/or iodine, for example chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, nonafluorobutoxy. Examples for C₁₀₆-haloalkoxy further encompass 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy and dodecafluorohexoxy. Particularly preferred are chloromethoxy, fluoromethoxy, difluoromethoxy, trifluoromethoxy, 2-fluoroethoxy, 2-chloroethoxy and 2,2,2-trifluoroethoxy.

The term “C₁-C₆-alkoxy-C₁-C₆-alkyl” as used herein refers to C₁-C₆-alkyl wherein 1 carbon atom carries a C₁-C₆-alkoxy radical as mentioned above. Examples are CH₂OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)-ethyl, 2-(1-methylethoxy)-ethyl, 2-(n-butoxy)ethyl, 2-(1-methylpropoxy)ethyl, 2-(2-methylpropoxy)-ethyl, 2-(1,1-dimethylethoxy)-ethyl, 2-(methoxy)-propyl, 2-(ethoxy)-propyl, 2-(n-propoxy)-propyl, 2-(1-methylethoxy)-propyl, 2-(n-butoxy)-propyl, 2-(1-methylpropoxy)-propyl, 2-(2-methylpropoxy)-propyl, 2-(1,1-dimethylethoxy)-propyl, 3-(methoxy)-propyl, 3-(ethoxy)-propyl, 3-(n-propoxy)-propyl, 3-(1-methylethoxy)-propyl, 3-(n-butoxy)-propyl, 3-(1-methylpropoxy)-propyl, 3-(2-methylpropoxy)-propyl, 3-(1,1-dimethylethoxy)-propyl, 2-(methoxy)-butyl, 2-(ethoxy)-butyl, 2-(n-propoxy)-butyl, 2-(1-methylethoxy)-butyl, 2-(n-butoxy)-butyl, 2-(1-methylpropoxy)-butyl, 2-(2-methylpropoxy)-butyl, 2-(1,1-dimethylethoxy)-butyl, 3-(methoxy)-butyl, 3-(ethoxy)-butyl, 3-(n-propoxy)-butyl, 3-(1-methylethoxy)-butyl, 3-(n-butoxy)-butyl, 3-(1-methylpropoxy)butyl, 3-(2-methylpropoxy)-butyl, 3-(1,1-dimethylethoxy)-butyl, 4-(methoxy)-butyl, 4-(ethoxy)-butyl, 4-(n-propoxy)-butyl, 4-(1-methylethoxy)-butyl, 4-(n-butoxy)-butyl, 4-(1-methylpropoxy)-butyl, 4-(2-methylpropoxy)-butyl, 4-(1,1-dimethylethoxy)-butyl and the like.

The term “(C₁-C₆-alkyl)carbonyl” as used herein refers to a straight-chain or branched saturated alkyl group having 1 to 6 carbon atoms, preferably 1 to 4 carbon atoms (=C₁-C₄-alkylcarbonyl) (as mentioned above) bonded via the carbon atom of the carbonyl group at any bond in the alkyl group. Examples for C₁-C₄-alkylcarbonyl include C(O)CH₃, C(O)C₂H₅, n-propylcarbonyl, 1-methylethylcarbonyl, n-butylcarbonyl, 1-methylpropylcarbonyl, 2-methylpropylcarbonyl and 1,1-dimethylethylcarbonyl. Examples for C₁-C₆-alkylcarbonyl further encompass n-pentylcarbonyl, 1-methylbutylcarbonyl, 2-methylbutylcarbonyl, 3-methylbutylcarbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl and the like.

The term “(C₁-C₆-haloalkyl)carbonyl” as used herein refers to a straight-chain or branched saturated haloalkyl group having 1 to 6 carbon atoms, preferably 1 to 4 carbon atoms (═C₁-C₄-haloalkylcarbonyl) (as mentioned above) bonded via the carbon atom of the carbonyl group at any bond in the haloalkyl group. Examples include chloromethylcarbonyl, bromomethylcarbonyl, dichloromethylcarbonyl, trichloromethylcarbonyl, fluoromethylcarbonyl, difluoromethylcarbonyl, trifluoromethylcarbonyl, chlorofluoromethylcarbonyl, dichlorofluoromethylcarbonyl, chlorodifluoromethylcarbonyl, 1-chloroethylcarbonyl, 1-bromoethylcarbonyl, 1-fluoroethylcarbonyl, 2-fluoroethylcarbonyl, 2,2-difluoroethylcarbonyl, 2,2,2-trifluoroethylcarbonyl, 2-chloro-2-fluoroethylcarbonyl, 2-chloro-2,2-difluoroethylcarbonyl, 2,2-dichloro-2-fluoroethylcarbonyl, 2,2,2-trichloroethylcarbonyl, pentafluoroethylcarbonyl and the like.

The term “(C₁-C₆-alkoxy)carbonyl” as used herein refers to a straight-chain or branched alkoxy group (as mentioned above) having 1 to 6 carbon atoms, preferably 1 to 4 carbon atoms (═C₁-C₄-alkoxycarbonyl) attached via the carbon atom of the carbonyl group (R—O—C(O)—; R=alkyl). Examples for C₁-C₄-alkoxycarbonyl include C(O)OCH₃, C(O)OC₂H₅, C(O)O—CH₂—C₂H₅, C(O)OCH(CH₃)₂, n-butoxycarbonyl, C(O)OCH(CH₃)—C₂H₅, C(O)OCH₂CH(CH₃)₂ and C(O)OC(CH₃)₃. Examples for C₁-C₆-alkoxycarbonyl further encompass n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl.

The term “(C₁-C₆-haloalkoxy)carbonyl” as used herein refers to a straight-chain or branched saturated haloalkoxy group having 1 to 6 carbon atoms, preferably 1 to 4 carbon atoms (═C₁-C₄-haloalkoxycarbonyl) (as mentioned above) bonded via the carbon atom of the carbonyl group (R—O—C(O)—; R=haloalkyl). Examples include chloromethoxycarbonyl, bromomethoxycarbonyl, dichloromethoxycarbonyl, trichloromethoxycarbonyl, fluoromethoxycarbonyl, difluoromethoxycarbonyl, trifluoromethoxycarbonyl, chlorofluoromethoxycarbonyl, dichlorofluoromethoxycarbonyl, chlorodifluoromethoxycarbonyl, 1-chloroethoxycarbonyl, 1-bromoethoxycarbonyl, 1-fluoroethoxycarbonyl, 2-fluoroethoxycarbonyl, 2,2-diffluoroethoxycarbonyl, 2,2,2-trifluoroethoxycarbonyl, 2-chloro-2-fluoroethoxycarbonyl, 2-chloro-2,2-difluoroethoxycarbonyl, 2,2-dichloro-2-fluoroethoxycarbonyl, 2,2,2-trichloroethoxycarbonyl, pentafluoroethoxycarbonyl and the like.

The term “(C₁-C₆-alkyl)carbonyloxy” as used herein refers to a straight-chain or branched saturated alkyl group having 1 to 6 carbon atoms, preferably 1 to 4 carbon atoms (═C₁-C₄-alkylcarbonyloxy) (as mentioned above) bonded via the carbon atom of the carbonyloxy group at any bond in the alkyl group. Examples for C₁-C₄-alkylcarbonyloxy include O—CO—CH₃, O—CO—C₂H₅, n-propylcarbonyloxy, 1-methylethylcarbonyloxy, n-butylcarbonyloxy, 1-methylpropylcarbonyloxy, 2-methylpropylcarbonyloxy and 1,1-dimethylethylcarbonyloxy. Examples for C₁-C₆-alkylcarbonyloxy further encompass n-pentylcarbonyloxy, 1-methylbutylcarbonyloxy, 2-methylbutylcarbonyloxy, 3-methylbutylcarbonyloxy, 1,1-dimethylpropylcarbonyloxy or 1,2-dimethylpropylcarbonyloxy.

The term “(C₁-C₆-haloalkyl)carbonyloxy” as used herein refers to a straight-chain or branched saturated haloalkyl group having 1 to 6 carbon atoms, preferably 1 to 4 carbon atoms (═C₁-C₄-haloalkylcarbonyloxy) (as mentioned above) bonded via the carbon atom of the carbonyloxy group at any bond in the haloalkyl group (R—C(O)—O—; R=haloalkyl). Examples for C₁-C₄-haloalkylcarbonyloxy include chloromethylcarbonyloxy, bromomethylcarbonyloxy, dichloromethylcarbonyloxy, trichloromethylcarbonyloxy, fluoromethylcarbonyloxy, difluoromethylcarbonyloxy, trifluoromethylcarbonyloxy, chlorofluoromethylcarbonyloxy, dichlorofluoromethylcarbonyoxyl, chlorodifluoromethylcarbonyloxy, 1-chloroethylcarbonyloxy, 1-bromoethylcarbonyloxy, 1-fluoroethylcarbonyloxy, 2-fluoroethylcarbonyloxy, 2,2-difluoroethylcarbonyloxy, 2,2,2-trifluoroethylcarbonyloxy, 2-chloro-2-fluoroethylcarbonyloxy, 2-chloro-2,2-difluoroethylcarbonyloxy, 2,2-dichloro-2-fluoroethylcarbonyloxy, 2,2,2-trichloroethylcarbonyloxy, pentafluoroethylcarbonyloxy and the like

The term “C₁-C₆-alkylthio “(C₁-C₆-alkylsulfanyl: C₁-C₆-alkyl-S—)” as used herein refers to a straight-chain or branched saturated alkyl group having 1 to 6 carbon atoms, preferably 1 to 4 carbon atoms (═C₁-C₄-alkylthio) (as mentioned above) which is attached via a sulfur atom. Examples for C₁-C₄-alkylthio include methylthio, ethylthio, propylthio, 1-methylethylthio, butylthio, 1-methylpropylthio, 2-methylpropylthio and 1,1-dimethylethylthio. Examples for C₁-C₆-alkylthio further encompass n-pentylthio, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylhio, 2,2-dimethylbutylthio, 2,3-dimethylbutylthio, 3,3-dimethylbutylthio, 1-ethylbutithio, 2-ethylbutylthio, 1,1,2-trimethylpropylthio, 1,2,2-trimethylpropylthio, 1-ethyl-1-methylpropylthio and 1-ethyl-2-methylpropylthio.

The term “C₁-C₆-haloalkylthio” as used herein refers to a C₁-C₆-alkylthio group as mentioned above wherein the hydrogen atoms are partially or fully substituted by fluorine, chlorine, bromine and/or iodine. Preferred are C₁-C₄-haloalkylthio groups, i.e. C₁-C₄-alkylthio groups as mentioned above wherein the hydrogen atoms are partially or fully substituted by fluorine, chlorine, bromine and/or iodine, for example chloromethylthio, dichloromethylthio, trichloromethylthio, fluoromethylthio, difluoromethylthio, trifluoromethylthio, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, pentafluoroethylthio, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio, 4-chlorobutylthio, 4-bromobutylthio, nonafluorobutylthio. Examples for C₁-C₆-haloalkylthio further encompass 5-fluoro-1-pentylthio, 5-chloro-1-pentylthio, 5-bromo-1-pentylthio, 5-iodo-1-pentylthio, 5,5,5-trichloro-1-pentylthio, undecafluoropentylthio, 6-fluoro-1-hexylthio, 6-chloro-1-hexylthio, 6-bromo-1-hexylthio, 6-iodo-1-hexylthio, 6,6,6-trichloro-1-hexylthio and dodecafluorohexylthio. Particularly preferred are chloromethylthio, fluoromethylthio, difluoromethylthio, trifluoromethylthio, 2-fluoroethylthio, 2-chloroethylthio and 2,2,2-trifluoroethylthio.

The term “(C₁-C₆-alkylthio)carbonyl” as used herein refers to a straight-chain or branched alkthio group (as mentioned above) having 1 to 6 carbon, preferably 1 to 4 carbon atoms (═C₁-C₄-alkylthiocarbonyl) atoms attached via the carbon atom of the carbonyl group. Examples for C₁-C₄-alkylthiocarbonyl include C(O)SCH₃, C(OSC₂H₅, C(O)—SCH₂—C₂H₅, C(O)SCH(CH₃)₂, n-butylthiocarbonyl, C(O)SCH(CH₃)—C₂H₅, C(O)SCH₂CH(CH₃)₂ and C(O)SC(CH₃)₃. Examples for C₁-C₆-alkylthiocarbonyl further encompass n-pentylthiocarbonyl, 1-methylbutylthiocarbonyl, 2-methylbutylthiocarbonyl, 3-methylbutylthiocarbonyl, 2,2-dimethylpropylthiocarbonyl, 1-ethylpropylthiocarbonyl, nhexylthiocarbonyl, 1,1-dimethylpropylthiocarbonyl, 1,2-dimethylpropylthiocarbonyl, 1-methylpentylthiocarbonyl, 2-methylpentylthiocarbonyl, 3-methylpentylthiocarbonyl, 4-methylpentylthiocarbonyl, 1,1-dimethylbutylthiocarbonyl, 1,2-dimethylbutylthiocarbonyl, 1,3-dimethylbutylhiocarbonyl, 2,2-dimethylbutylthiocarbonyl, 2,3-dimethylbutylthiocarbonyl, 3,3-dimethylbutylthiocarbonyl, 1-ethylbutithioycarbonyl, 2-ethylbutylthiocarbonyl, 1,1,2-trimethylpropylthiocarbonyl, 1,2,2-trimethylpropylthiocarbonyl, 1-ethyl-1-methylpropylthiocarbonyl and 1-ethyl-2-methylpropylthiocarbonyl.

The term “(C₁-C₆-alkoxy)thiocarbonyl” as used herein refers to a straight-chain or branched alkoxy group (as mentioned above) having 1 to 6 carbon atoms, preferably 1 to 4 carbon atoms (═C₁-C₄-alkoxythiocarbonyl) attached via the carbon atom of the carbonyl group. Examples for C₁-C₄-alkoxythiocarbonyl include C(S)OCH₃, C(S)OC₂H₅, C(S)O—CH₂—C₂H₅, C(S)OCH(CH₃)₂, n-butoxythiocarbonyl, C(S)OCH(CH₃)—C₂H₅, C(S)OCH₂CH(CH₃)₂ and C(S)OC(CH₃)₃. Examples for C₁-C₆-alkoxythiocarbonyl further encompass n-pentoxythiocarbonyl, 1-methylbutoxythiocarbonyl, 2-methylbutoxythiocarbonyl, 3-methylbutoxythiocarbonyl, 2,2-dimethylpropoxythiocarbonyl, 1-ethylpropoxythiocarbonyl, n-hexoxythiocarbonyl, 1,1-dimethylpropoxythiocarbonyl, 1,2-dimethylpropoxythiocarbonyl, 1-methylpentoxythiocarbonyl, 2-methylpentoxythiocarbonyl, 3-methylpentoxythiocarbonyl, 4-methylpentoxythiocarbonyl, 1,1-dimethylbutoxythiocarbonyl, 1,2-dimethylbutoxythiocarbonyl, 1,3-dimethylbutoxythiocarbonyl, 2,2-dimethylbutoxythiocarbonyl, 2,3-dimethylbutoxythiocarbonyl, 3,3-dimethylbutoxythiocarbonyl, 1-ethylbutoxythiocarbonyl, 2-ethylbutoxythiocarbonyl, 1,1,2-trimethylpropoxythiocarbonyl, 1,2,2-trimethylpropoxythiocarbonyl, 1-ethyl-1-methylpropoxythiocarbonyl or 1-ethyl-2-methylpropoxythiocarbonyl.

The term “C₁-C₆-alkylsulfinyl” (C₁-C₆-alkylsulfoxyl: C₁-C₆-alkyl-S(═O)—), as used herein refers to a straight-chain or branched saturated alkyl group (as mentioned above) having 1 to 6 carbon atoms, preferably 1 to 4 carbon atoms (═C₁-C₄-alkylsulfinyl) bonded through the sulfur atom of the sulfinyl group at any position in the alkyl group. Examples for C₁-C₄-alkylsulfinyl include S(O)CH₃, S(O)C₂H₅, n-propylsulfinyl, 1-methylethylsulfinyl, n-butylsulfinyl, 1-methylpropylsulfinyl, 2-methylpropylsulfinyl and 1,1-dimethylethylsulfinyl. Examples for C₁-C₆-alkylsulfinyl further encompass n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl and 1-ethyl-2-methylpropylsulfinyl.

The term “C₁-C₆-alkylsulfonyl” (C₁-C₆-alkyl-S(═O)₂—) as used herein refers to a straight-chain or branched saturated alkyl group having 1 to 6 carbon atoms, preferably 1 to 4 carbon atoms (═C₁-C₄-alkylsulfonyl) (as mentioned above) which is bonded via the sulfur atom of the sulfonyl group at any position in the alkyl group. Examples for C₁-C₄-alkylsulfonyl include SO₂—CH₃, SO₂—C₂H₅, n-propylsulfonyl, SO₂—CH(CH₃)₂, n-butylsulfonyl, 1-methylpropylsulfonyl, 2-methylpropylsulfonyl and SO₂—C(CH₃)₃. Examples for C₁-C₆-alkylsulfonyl further encompass n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, nhexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl and 1-ethyl-2-methylpropylsulfonyl.

The term “C₁-C₆-haloalkylsulfonyl” (C₁-C₆-haloalkyl-S(═O)₂—) as used herein refers to a straight-chain or branched saturated haloalkyl group having 1 to 6 carbon atoms, preferably 1 to 4 carbon atoms (═C₁-C₄-haloalkylsulfonyl) (as mentioned above) which is bonded via the sulfur atom of the sulfonyl group at any position in the alkyl group. Examples for C₁-C₄-haloalkylsulfonyl include chloromethylsulfonyl, dichloromethylsulfonyl, trichloromethylsulfonyl, fluoromethylsulfonyl, difluoromethylsulfonyl, trifluoromethylsulfonyl, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, pentafluoroethylsulfonyl, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, 2,2,3,3,3-pentafluoropropylsulfonyl, heptafluoropropylsulfonyl, 1-(fluoromethyl)-2-fluoroethylsulfonyl, 1-(chloromethyl)-2-chloroethylsulfonyl, 1-(bromomethyl)-2-bromoethylsulfonyl, 4-fluorobutylsulfonyl, 4-chlorobutylsulfonyl, 4-bromobutylsulfonyl, nonafluorobutylsulfonyl. Examples for C₁-C₆-haloalkylsulfonyl further encompass 5-fluoro-1-pentylsulfonyl, 5-chloro-1-pentylsulfonyl, 5-bromo-1-pentylsulfonyl, 5-iodo-1-pentylsulfonyl, 5,5,5-trichloro-1-pentylsulfonyl, undecafluoropentylsulfonyl, 6-fluoro-1-hexylsulfonyl, 6-chloro-1-hexylsulfonyl, 6-bromo-1-hexylsulfonyl, 6-iodo-1-hexylsulfonyl, 6,6,6-trichloro-1-hexylsulfonyl and dodecafluorohexylsulfonyl. Particularly preferred are chloromethylsulfonyl, fluoromethylsulfonyl, difluoromethylsulfonyl, trifluoromethylsulfonyl, 2-fluoroethylsulfonyl, 2-chloroethylsulfonyl and 2,2,2-trifluoroethylsulfonyl.

The term “C₁-C₆-alkylamino” refers to a secondary amino group carrying one alkyl group as defined above, e.g. methylamino, ethylamino, propylamino, 1-methylethylamino, butylamino, 1-methylpropylamino, 2-methylpropylamino, 1,1-dimethylethylamino, pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino, 3,3-dimethylbutylamino, 1-ethylbutylamino, 2-ethylbutylamino, 1,1,2-trimethylpropylamino, 1,2,2-trimethylpropylamino, 1-ethyl-1-methylpropylamino or 1-ethyl-2-methylpropylamino.

The term “di(C₁-C₆-alkyl)amino)” refers to a tertiary amino group carrying two alkyl radicals as defined above, e.g. dimethylamino, diethylamino, di-n-propylamino, diisopropylamino, N-ethyl-N-methylamino, N-(n-propyl)-N-methylamino, N-(isopropyl)N-methylamino, N-(n-butyl)-N-methylamino, N-(n-pentyl)-N-methylamino, N-(2-butyl)N-methylamino, N-(isobutyl)-N-methylamino, N-(n-pentyl)-N-methylamino, N-(n-propyl)N-ethylamino, N-(isopropyl)-N-ethylamino, N-(n-butyl)-N-ethylamino, N-(n-pentyl)N-ethylamino, N-(2-butyl)-N-ethylamino, N-(isobutyl)-N-ethylamino or N-(n-pentyl)N-ethylamino.

The term “C₂-C₆-alkenyl” as used herein and in the alkenyl moieties of C₂-C₆-alkenyloxy, C₂-C₆-alkenylamino, C₂-C₆-alkenylthio, C₂-C₆-alkenylsulfonyl, (C₂-C₆-alkenyl)carbonyl, (C₂-C₆-alkenyloxy)carbonyl and (C₂-C₆-alkenyl)carbonyloxy refers to a straight-chain or branched unsaturated hydrocarbon group having 2 to 6 carbon atoms and a double bond in any position, such as ethenyl, 1-propenyl, 2-propenyl, 1-methyl-ethenyl, 1-butenyl, 2-butenyl, 3-butenyl, 1-methyl-1-propenyl, 2-methyl-1-propenyl, 1-methyl-2-propenyl, 2-methyl-2-propenyl; 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term, “C₂-C₆-alkenyloxy” as used herein refers to a straight-chain or branched alkenyl group having 2 to 6 carbon atoms (as mentioned above) which is attached via an oxygen atom, such as vinyloxy, allyloxy (propen-3-yloxy), methallyloxy, buten-4-yloxy, etc.

The term “C₂-C₆-alkenylthio” as used herein refers to a straight-chain or branched alkenyl group having 2 to 6 carbon atoms (as mentioned above) which is attached via a sulfur atom, for example vinylsulfanyl, allylsulfanyl (propen-3-ylthio), methallylsufanyl, buten-4-ylsulfanyl, etc.

The term “C₂-C₆-alkenylcarbonyl” as used herein refers to a straight-chain or branched alkenyl group having 2 to 6 carbon atoms (as mentioned above) which is bonded via the carbon atom of the carbonyl group at any bond in the alkenyl group, for example vinylcarbonyl, allylcarbonyl (propen-3-ylcarbonyl), methallylcarbonyl, buten-4-ylcarbonyl, etc.

The term “C₂-C₆-alkenyloxycarbonyl” as used herein refers to a straight-chain or branched alkenyl group having 2 to 6 carbon atoms (as mentioned above) which is bonded via the carbon atom of the oxycarbonyl group (RO—C(O)—; R═C₂-C₆-alkenyl), for example vinyloxycarbonyl, allyloxycarbonyl (propen-3-yloxycarbonyl), methallyloxycarbonyl, buten-4-yloxycarbonyl, etc.

The term “C₂-C₆-alkenylcarbonyloxy” as used herein refers to a straight-chain or branched alkenyl group having 2 to 6 carbon atoms (as mentioned above) which is bonded via the oxygen atom of the carbonyloxy group (R—C(O)—O—; R═C₂-C₆-alkenyl), for example vinylcarbonyloxy, allylcarbonyloxy (propen-3-ylcarbonyloxy), methallylcarbonyloxy, buten-4-ylcarbonyloxy, etc.

The term “C₂-C₆-alkenylamino” as used herein refers to a straight-chain or branched alkenyl group having 2 to 6 carbon atoms (as mentioned above) which is attached via a sulfur atom, for example vinylamino, allylamino (propen-3-ylamino), methallylamino, buten-4-ylamino, etc.

The term “C₂-C₆-alkenylsulfonyl” as used herein refers to a straight-chain or branched alkenyl group having 2 to 6 carbon atoms (as mentioned above) which is attached via a sulfonyl (SO₂) group, for example vinylsulfonyl, allylsulfonyl (propen-3-ylsulfonyl), methallylsulfonyl, buten-4-ylsulfonyl, etc.

The term “C₂-C₆-alkynyl” as used herein and in the alkynyl moieties of C₂-C₆-alkynyloxy, C₂-C₆-alkynylamino, C₂-C₆-alkynylthio, C₂-C₆-alkynylsulfonyl, C₂-C₆-alkynylcarbonyl, C₂-C₆-alkynyloxycarbonyl and C₁-C₆-alkynylcarbonyloxy refers to a straight-chain or branched unsaturated hydrocarbon group having 2 to 6 carbon atoms and containing at least one triple bond, such as ethynyl, prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl and the like.

The term, “C₂-C₆-alkynyloxy” as used herein refers to a straight-chain or branched alkynyl group having 2 to 6 carbon atoms (as mentioned above) which is attached via an oxygen atom, such as propargyloxy (propyn-3-yloxy), butyn-3-yloxy, and butyn-4-yloxy.

The term “C₂-C₆-alkynylthio” as used herein refers to a straight-chain or branched alkynyl group having 2 to 6 carbon atoms (as mentioned above) which is attached via a sulfur atom, such as propargylsulfanyl (propyn-3-ylthio), butyn-3-ylsufanyl and butyn-4-ylsulfanyl.

The term “C₂-C₆-alkynylcarbonyl” as used herein refers to a straight-chain or branched alkynyl group having 2 to 6 carbon atoms (as mentioned above) which is bonded via the carbon atom of the carbonyl group at any bond in the alkynyl group, for example propargylcarbonyl (propyn-3-ylcarbonyl), butyn-3-ylcarbonyl, and butyn-4-ylcarbonyl.

The term “C₂-C₆-alkynyloxycarbonyl” as used herein refers to a straight-chain or branched alkynyl group having 2 to 6 carbon atoms (as mentioned above) which is bonded via the carbon atom of the oxycarbonyl group (RO—C(O)—; R═C₂-C₆-alkynyl), for example propargyloxycarbonyl (propyn-3-yloxycarbonyl), butyn-3-yloxycarbonyl, and butyn-4-yloxycarbonyl.

The term “C₂-C₆-alkynylcarbonyloxy” as used herein refers to a straight-chain or branched alkynyl group having 2 to 6 carbon atoms (as mentioned above) which is bonded via the oxygen atom of the carbonyloxy group (R—C(O)—O—; R═C₂-C₆-alkynyl), for example propargylcarbonyloxy (propyn-3-ylcarbonyloxy), butyn-3-ylcarbonyloxy, and butyn-4-ylcarbonyloxy.

The term “C₂-C₆-alkynylamino” as used herein refers to a straight-chain or branched alkynyl group having 2 to 6 carbon atoms (as mentioned above) which is attached via a sulfur atom, such as propargylamino (propyn-3-ylamino), butyn-3-amino, and butyn-4-ylamino.

The term “C₂-C₆-alkynylsulfonyl” as used herein refers to a straight-chain or branched alkynyl group having 2 to 6 carbon atoms (as mentioned above) which is attached via a sulfonyl (SO₂) group, such as propargylsulfonyl (propin-3-yltsulfonyl), butin-3-ylsulfonyl and butin-4-ylsulfonyl.

The term “C₃-C₁₂-cycloalkyl” as used herein refers to a mono- or bi- or polycyclic hydrocarbon radical having 3 to 8 carbon atoms (═C₃-C₈-cycloalkyl), in particular 3 to 6 carbon atoms (═C₃-C₆-cycloalkyl). Examples of monocyclic radicals comprise cyclopropyl, cyclobutyl, cyclopentyl, cyclohexyl, cycloheptyl, cyclooctyl, cyclononyl and cyclodecyl. Examples of bicyclic radicals comprise bicyclo[2.2.1]heptyl, bicyclo[3.1.1]heptyl, bicyclo[2.2.2]octyl and bicyclo[3.2.1]octyl.

The term “C₃-C₆-halocycloalkyl” as used herein refers to a monocyclic hydrocarbon radical having 3 to 6 carbon atoms, wherein the hydrogen atoms are partially or fully substituted by fluorine, chlorine, bromine and/or iodine. Examples are 1- and 2-fluorocyclopropyl, 1,2-, 2,2- and 2,3-difluorocyclopropyl, 1,2,2-trifluorocyclopropyl, 2,2,3,3-tetrafluorocyclpropyl, 1- and 2-chlorocyclopropyl, 1,2-, 2,2- and 2,3-dichlorocyclopropyl, 1,2,2-trichlorocyclopropyl, 2,2,3,3-tetrachlorocyclpropyl, 1-, 2- and 3-fluorocyclopentyl, 1,2-, 2,2-, 2,3-, 3,3-, 3,4-, 2,5-difluorocyclopentyl, 1-, 2- and 3-chlorocyclopentyl, 1,2-, 2,2-, 2,3-, 3,3-, 3,4-, 2,5-dichlorocyclopentyl and the like.

The term “C₃-C₆-cycloalkoxy” as used herein refers to a monocyclic hydrocarbon radical having 3 to 6 carbon atoms which is bound via an oxygen atom. Examples include cyclopropyloxy, cyclobutyloxy, cyclopentyloxy and cyclohexyloxy.

The term “aryl” as used herein refers to a 06-014 carboaromatic group, such as phenyl, Naphthyl, anthracenyl and phenanthrenyl. Preferably, aryl is phenyl.

The term “aryl-C₁-C₄-alkyl” as used herein refers to aryl as defined above which is bound via a C₁-C₄-alkyl group, in particular a methyl or an ethyl group, to the remainder of the molecule. Examples are benzyl, 2-phenylethyl, naphthylmethyl and the like.

The term “aryl-C₁-C₄-alkoxy” as used herein refers to aryl-C₁-C₄-alkyl as defined above which is bound via an oxygen atom to the remainder of the molecule (aryl-C₁-C₄-alkyl-O—). One example is benzoxy.

The term “aryloxy” refers to aryl as defined above which is bound via an oxygen atom to the remainder of the molecule. Examples are phenoxy and naphthoxy.

The term “arylcarbonyl” refers to aryl as defined above which is bound via the C atom of a carbonyl group to the remainder of the molecule. Examples are benzoyl and naphthylcarbonyl.

The term “aryloxycarbonyl” refers to aryl as defined above which is bound via the C atom of an oxycarbonyl group to the remainder of the molecule (Ar—O—C(O)—; Ar=hetaryl).

Phenyl fused to phenyl is naphthyl.

Phenyl fused to a 5- or 6-membered non-aromatic (i.e. saturated or partially unsaturated) heterocyclic ring is for example 2,3-dihydrobenzofuranyl, benzoxolanyl, 2,3-dihydrobenzothienyl, indolinyl, chromanyl, chromenyl, benzodioxanyl and the like. Examples for phenyl fused to a 5- or 6-membered aromatic heterocyclic ring (=fused to a 5- or 6-membered heteroaromatic ring) are given below.

The term “hetaryl/heteroaromatic ring” as used herein refers to a monocyclic heteroaromatic radical which has 5 or 6 ring members, which may be fused to a carbocyclic or heterocyclic 5, 6 or 7 membered ring thus having a total number of ring members from 8 to 10, wherein in each case 1, 2, 3 or 4, preferably 1, 2 or 3, of these ring members are heteroatoms selected, independently from each other, from the group consisting of oxygen, nitrogen and sulfur. The heterocyclic radical may be attached to the remainder of the molecule via a carbon ring member or via a nitrogen ring member. The carbocyclic or heterocyclic fused ring is selected from C₅-C₇-cycloalkyl, C₅-C₇-cycloalkenyl, 5 to 7 membered heterocyclyl and phenyl.

Examples for monocyclic 5- to 6-membered heteroaromatic rings include triazinyl, pyrazinyl, pyrimidyl, pyridazinyl, pyridyl, thienyl, furyl, pyrrolyl, pyrazolyl, imidazolyl, triazolyl, tetrazolyl, thiazolyl, oxazolyl, thiadiazolyl, oxadiazolyl, isothiazolyl and isoxazolyl.

Examples for 5- to 6-membered heteroaromatic rings being fused to a phenyl ring (or for a phenyl ring fused to a 5- to 6-membered heteroaromatic ring) are quinolinyl, isoquinolinyl, indolyl, indolizinyl, isoindolyl, indazolyl, benzofuryl, benzthienyl, benzo[b]thiazolyl, benzoxazolyl, benzthiazolyl, benzoxazolyl, and benzimidazolyl. Examples for 5- to 6-membered heteroaromatic rings being fused to a cycloalkenyl ring are dihydroindolyl, dihydroindolizinyl, dihydroisoindolyl, dihydrochinolinyl, dihydroisochinolinyl, chromenyl, chromanyl and the like.

The term “hetaryl-C₁-C₄-alkyl” refers to hetaryl as defined above which is bound via a C₁-C₄-alkyl group, in particular a methyl group (=hetarylmethyl), to the remainder of the molecule.

The term “hetaryloxy” refers to hetaryl as defined above which is bound via an oxygen atom to the remainder of the molecule.

The term “hetarylcarbonyl” refers to hetaryl as defined above which is bound via the C atom of a carbonyl group to the remainder of the molecule.

The term “hetaryloxycarbonyl” refers to hetaryl as defined above which is bound via the C atom of an oxycarbonyl group to the remainder of the molecule (Het-O—C(O)—; Het=hetaryl).

The term “hetarylmethylcarbonyl” refers to hetarylmethyl as defined above which is bound via the C atom of a carbonyl group to the remainder of the molecule (Het-CH₂—C(O)—; Het=hetaryl).

The term “(saturated, partially unsaturated or aromatic) 5 or 6-membered heterocyclic ring/heterocyclyl” comprises heteroaromatic rings as defined above and nonaromatic saturated or partially unsaturated heterocyclic rings having 5 or 6 ring members and 1, 2, 3 or 4, preferably 1, 2 or 3 heteroatoms as ring members. The heterocyclic radical may be attached to the remainder of the molecule via a carbon ring member or via a nitrogen ring member. Examples for non-aromatic rings include pyrrolidinyl, pyrazolinyl, imidazolinyl, pyrrolinyl, pyrazolinyl, imidazolinyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperazinyl, pyranyl, dihydropyranyl, tetrahydropyranyl, 1,3- and 1,4-dioxanyl, thiopyranyl, dihydrothiopyranyl, tetrahydrothiopyranyl, morpholinyl, thiazinyl and the like. Examples for heterocyclic ring also comprising 1 or 2 carbonyl groups as ring members comprise pyrrolidin-2-only, pyrrolidin-2,5-dionyl, imidazolidin-2-only, oxazolidin-2-only, thiazolidin-2-only and the like.

The term “5-, 6- or 7-membered carbocycle” comprises monocyclic aromatic rings and nonaromatic saturated or partially unsaturated carbocyclic rings having 5, 6 or 7 ring members. Examples for non-aromatic rings include cyclopentyl, cyclopentenyl, cyclopentadienyl, cyclohexyl, cyclohexenyl, cyclohexadienyl, cycloheptyl, cycloheptenyl, cycloheptadienyl and the like.

The term “linear (C₁-C₆)-alkandiyl” as used herein refers to methylendiyl, ethane-1,2-diyl, propane-1,3-diyl, butane-1,4-diyl, pentane-1,5-diyl, hexane-1,6-diyl.

The term “(C₂-C₅)-alkandiyl” as used herein refers to alkylene groups such as ethane-1,2-diyl, propane-1,3-diyl, butane-1,4-diylor pentane-1,5-diyl, where a part or all hydrogen atoms of the alkylene groups may be substituted as defined above. If one or two of the methylene groups are replaced by O, S or NR, the term also encompasses for example —O—CH₂—, —S—CH₂—, —NR—CH₂—, —O—CH₂—CH₂—, —S—CH₂—CH₂—, —NR—CH₂—CH₂—, —CH₂—O—CH₂—, —CH₂—S—CH₂—, —CH₂—NR—CH₂—, —O—CH₂—O—, —S—CH₂—S—, —NR—CH₂—NR—, —O—CH₂—CH₂—CH₂—, —S—CH₂—CH₂—CH₂—, —NR—CH₂—CH₂—CH₂—, —CH₂—O—CH₂—CH₂—, —CH₂—S—CH₂—CH₂—, —CH₂—NR—CH₂—CH₂—, —O—CH₂—CH₂—O—, —S—CH₂—CH₂—S—, —NR—CH₂—CH₂—NR—, —O—CH₂—CH₂—CH₂—CH₂—, —S—CH₂—CH₂—CH₂—CH₂—, —NR—CH₂—CH₂—CH₂—CH₂—, —CH₂—O—CH₂—CH₂—CH₂—, —CH₂—S—CH₂—CH₂—CH₂—, —CH₂—NR—CH₂—CH₂—CH₂—, —CH₂—CH₂—O—CH₂—CH₂—, —CH₂—CH₂—S—CH₂—CH₂—, —CH₂—CH₂—NR—CH₂—CH₂—, —O—CH₂—CH₂—CH₂—O—, —S—CH₂—CH₂—CH₂—S—, —NR—CH₂—CH₂—CH₂—NR— and the like. Here, too, a part or all hydrogen atoms of the methylene groups may be substituted as defined above.

The remarks made below as to preferred embodiments of the variables of the compounds (I), of the features of the use and method according to the invention and of the composition of the invention are valid on their own as well as—preferably—in combination with each other.

R¹ is preferably selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₃-C₆-cycloalkyl, phenyl, benzyl, phenoxycarbonyl, 5- or 6-membered hetaryl and 5- or 6-membered hetarylmethyl each of the six last mentioned radicals may be unsubstituted or may carry any combination of 1, 2, 3, 4 or 5 radicals R^{b1}.

More preferably, R¹ is selected from the group consisting of hydrogen, C₁-C₆-alkyl, C₁-C₆-haloalkyl, C₂-C₆-alkenyl, C₂-C₆-alkynyl, phenyl, benzyl, 5- or 6-membered hetaryl and 5- or 6-membered hetarylmethyl, where each of the last four mentioned radicals may be unsubstituted or may carry any combination of 1, 2, 3, 4 or 5, preferably 1, 2 or 3, more preferably 1 or 2 radicals R^{b1}.

Even more preferably, R¹ is selected from the group consisting of hydrogen, C₁-C₆-alkyl, phenyl and benzyl, where the phenyl moiety in the 2 last-mentioned radicals may carry 1, 2, 3, 4 or 5, preferably 1, 2 or 3, more preferably 1 or 2 radicals R^{b1}.

In a particularly preferred embodiment, R¹ is hydrogen.

In an alternatively preferred embodiment, R¹ is different from hydrogen. Particularly, R¹ is in this case selected from C₁-C₆-alkyl, C₁-C₆-haloalkyl, C₂-C₆-alkenyl, C₂-C₆-alkynyl, phenyl, benzyl, 5- or 6-membered hetaryl and 5- or 6-membered hetarylmethyl, where each of the last four mentioned radicals may be unsubstituted or may carry any combination of 1, 2, 3, 4 or 5, preferably 1, 2 or 3, more preferably 1 or 2 radicals R^{b1}. More particularly, R¹ is in this case selected from C₁-C₆-alkyl, phenyl and benzyl, where the phenyl moiety in the 2 last-mentioned radicals may carry 1, 2, 3, 4 or 5, preferably 1, 2 or 3, more preferably 1 or 2 radicals R^{b1}.

R^{b1} is preferably selected from halogen, hydroxy, amino, C₁-C₆-alkyl, C₁-C₆-haloalkyl, C₁-C₆-alkoxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylcarbonyl, C₁-C₆-alkoxycarbonyl and C₁-C₆-alkylcarbonyloxy and more preferably from halogen, C₁-C₆-alkyl, C₁-C₆-haloalkyl, C₁-C₆-alkoxy and C₁-C₆-haloalkoxy.

The variables R^{2a} or R^{2b} are preferably selected from the group consisting of hydrogen, C₁-C₄-alkyl, formyl, CN, C(S)NR^{a}R^{b}, C₁-C₆-alkylcarbonyl, C₁-C₄-haloalkylcarbonyl, C₁-C₆-alkoxycarbonyl, C₁-C₄-alkoxy-C₁-C₄-alkoxycarbonyl, C₁-C₆-alkylthiocarbonyl, benzoyl, 5 or 6 membered hetarylcarbonyl, each of the last two mentioned radicals may be unsubstituted or may carry any combination of 1, 2 or 3 R^{b2}. More preferably R^{2a} or R^{2b} is hydrogen. In another preferred embodiment R^{2a} and R^{2b} are selected from the group consisting of oxazolyl, thiazolyl and imidazolyl.

Independently of each other, the variables R^{2a} and R^{2b} are preferably selected from hydrogen, C₁-C₆-alkyl, formyl, CN, C(S)NR^{a}R^{b}, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl, C₁-C₆-alkylthiocarbonyl, phenyl, benzoyl, benzyl, benzylcarbonyl, a 5- or 6-membered saturated, partially unsaturated or aromatic heterocyclic ring containing 1, 2 or 3 heteroatoms selected from O, S and N as ring members, hetarylmethyl, hetarylcarbonyl and hetarylmethylcarbonyl, wherein each of the 8 last mentioned radicals may be unsubstituted or may carry any combination of 1, 2 or 3 radicals R^{b2}, and wherein the heteroaromatic ring in hetarylmethyl, hetarylcarbonyl and hetarylmethylcarbonyl is 5- or 6-membered and contains 1, 2, 3 or 4, preferably 1, 2 or 3 heteroatoms selected from oxygen, sulfur and nitrogen as ring members.

More preferably, R^{2a} and R^{2b} are selected from hydrogen, C₁-C₆-alkyl, formyl, CN, C(S)NR^{a}R^{b}, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl, C₁-C₆-alkylthiocarbonyl, benzoyl, benzylcarbonyl, a 5- or 6-membered saturated, partially unsaturated or aromatic heterocyclic ring containing 1, 2 or 3 heteroatoms selected from O, S and N as ring members, hetarylcarbonyl and hetarylmethylcarbonyl, wherein each of the 5 last mentioned radicals may be unsubstituted or may carry any combination of 1, 2 or 3 radicals R^{b2}, and wherein the heteroaromatic ring in hetarylcarbonyl and hetarylmethylcarbonyl is 5- or 6-membered and contains 1, 2, 3 or 4, preferably 1, 2 or 3 heteroatoms selected from oxygen, sulfur and nitrogen as ring members.

In an even more preferred embodiment, R^{2a} is selected from hydrogen, C₁-C₆-alkyl, formyl, CN, C(S)NR^{a}R^{b}, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl, C₁-C₆-alkylthiocarbonyl, benzoyl, benzylcarbonyl, hetarylcarbonyl and hetarylmethylcarbonyl, wherein each of the 4 last mentioned radicals may be unsubstituted or may carry any combination of 1, 2 or 3 radicals R^{b2}, and wherein the heteroaromatic ring in hetarylcarbonyl and hetarylmethylcarbonyl is 5- or 6-membered and contains 1, 2, 3 or 4, preferably 1, 2 or 3 heteroatoms selected from oxygen, sulfur and nitrogen as ring members.

Particularly, R^{2a} is selected from hydrogen, C₁-C₆-alkyl, CN, C₁-C₆-alkylcarbonyl, C₁-C₆-haloalkylcarbonyl, benzoyl and hetarylcarbonyl, wherein the 2 last mentioned radicals may be unsubstituted or may carry any combination of 1, 2 or 3 radicals R^{b2}, and wherein the heteroaromatic ring in hetarylcarbonyl is 5- or 6-membered and contains 1, 2, 3 or 4, preferably 1, 2 or 3 heteroatoms selected from oxygen, sulfur and nitrogen as ring members. Specifically, R^{2a} is hydrogen or C₁-C₆-alkyl, and more specifically hydrogen.

In an even more preferred embodiment, R^{2b} is selected from hydrogen, C₁-C₆-alkyl, formyl, CN, C(S)NR^{a}R^{b}, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl, C₁-C₆-alkylthiocarbonyl, benzoyl, benzylcarbonyl, a 5- or 6-membered saturated, partially unsaturated or aromatic heterocyclic ring containing 1, 2 or 3 heteroatoms selected from O, S and N as ring members, hetarylcarbonyl and hetarylmethylcarbonyl, wherein each of the 5 last mentioned radicals may be unsubstituted or may carry any combination of 1, 2 or 3 radicals R^{b2}, and wherein the heteroaromatic ring in hetarylcarbonyl and hetarylmethylcarbonyl is 5- or 6-membered and contains 1, 2, 3 or 4, preferably 1, 2 or 3 heteroatoms selected from oxygen, sulfur and nitrogen as ring members.

Particularly, R^{2b} is selected from hydrogen, C₁-C₆-alkyl, CN, C₁-C₆-alkylcarbonyl, C₁-C₆-haloalkylcarbonyl, benzoyl, hetarylcarbonyl, wherein the 2 last mentioned radicals may be unsubstituted or may carry any combination of 1, 2 or 3 radicals R^{b2}, and wherein the heteroaromatic ring in hetarylcarbonyl is 5- or 6-membered and contains 1, 2, 3 or 4, preferably 1, 2 or 3 heteroatoms selected from oxygen, sulfur and nitrogen as ring members; and a 5- or 6-membered saturated, partially unsaturated or aromatic heterocyclic ring containing 1, 2 or 3 heteroatoms selected from O, S and N as ring members which may carry any combination of 1, 2 or 3 radicals R^{b2}. More particularly, R^{2b} is selected from hydrogen, C₁-C₆-alkyl and a 5- or 6-membered saturated, partially unsaturated or aromatic heterocyclic ring containing 1, 2 or 3 heteroatoms selected from O, S and N as ring members which may carry any combination of 1, 2 or 3 radicals R^{b2}. Even more particularly, R^{2b} is H or a 5- or 6-membered saturated, partially unsaturated or aromatic heterocyclic ring containing 1, 2 or 3 heteroatoms selected from O, S and N as ring members which may carry any combination of 1, 2 or 3 radicals R^{b2}. Specifically, R^{2b} is a 5- or 6-membered saturated, partially unsaturated or aromatic heterocyclic ring containing 1, 2 or 3 heteroatoms selected from O, S and N as ring members which may carry any combination of 1, 2 or 3 radicals R^{b2}.

The 5- or 6-membered heterocyclic ring R^{2b} is preferably non-aromatic, i.e. saturated or partially unsaturated, and more preferably partially unsaturated. Preferably, the 5- or 6-membered heterocyclic ring R^{2b} is unsubstituted. Preferably, the 5- or 6-membered heterocyclic ring R^{2b} is 5-membered and contains two heteroatoms. More preferably, the 5- or 6-membered heterocyclic ring R^{2b} is selected from oxazolinyl and thiazolinyl, more preferably oxazolinyl and thiazolinyl which are bound in the 2-position (with respect to the 1-position of O and S, respectively, and to the 3-position of N), and specifically R^{2b} is thiazolinyl which is preferably bound in the 2-position.

R^{b2} is preferably selected from halogen, hydroxy, amino, C₁-C₆-alkyl, C₁-C₆-haloalkyl, C₁-C₆-alkoxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylcarbonyl, C₁-C₆-alkoxycarbonyl and C₁-C₆-alkylcarbonyloxy and more preferably from halogen, C₁-C₆-alkyl, C₁-C₆-haloalkyl, C₁-C₆-alkoxy and C₁-C₆-haloalkoxy.

In another preferred embodiment of the invention the radicals R¹ and R^{2b} together form a bridging bivalent carbonyl group C(O).

R^{2c} and R^{2d}, independently of each other, are preferably selected from hydrogen, formyl, C₁-C₆-alkyl, C₁-C₆-alkylcarbonyl, C₁-C₆-alkylthiocarbonyl, C₁-C₆-alkoxycarbonyl, C₁-C₆-alkoxythiocarbonyl, arylcarbonyl, hetarylcarbonyl, aryloxycarbonyl and hetaryloxycarbonyl, where the aliphatic, aromatic or heteroaromatic moieties in the 9 last-mentioned radicals may carry 1, 2, 3, 4 or 5, preferably 1, 2 or 3 radicals R^{c2}, and where hetaryl is a 5- or 6-membered heteroaromatic ring containing 1, 2, 3 or 4 heteroatoms selected from O, S and N as ring members. More preferably, R^{2c} and R^{2d} are hydrogen.

Among compounds of general formula (I) preference is given to compounds wherein each of the radicals R^{3a}, R^{3b}, R^{3c} and R^{3d} is hydrogen.

Preference is given to compounds of general formula (I) wherein the radicals R^{4a}, R^{4b}, R^{4c} and R^{4d} are independently selected from the group consisting of hydrogen, halogen, C₁-C₆-alkyl, C₁-C₆-haloalkyl, C₂-C₆-alkenyl, C₂-C₆-alkynyl, phenyl, 5- or 6-membered hetaryl, 5- or 6-membered hetarylmethyl and benzyl wherein the four last mentioned radicals may be unsubstituted or may carry any combination of 1, 2, 3, 4 or 5 radicals R^{b4}.

More preferably, R^{4a}, R^{4b}, R^{4c} and R^{4d} are independently selected from the group consisting of hydrogen, halogen, C₁-C₆-alkyl, C₁-C₆-haloalkyl, phenyl and benzyl wherein the phenyl moiety in the 2 last mentioned radicals may be unsubstituted or may carry any combination of 1, 2, 3, 4 or 5, preferably 1, 2 or 3 radicals R^{b4}.

In a particular embodiment of the invention, all radicals R^{4a}, R^{4b}, R^{4c} and R^{4d} are hydrogen.

In an alternative particular embodiment, at least one of the radicals R^{4a}, R^{4b}, R^{4c} and R^{4d} is different from hydrogen.

In this particular embodiment, R^{4a} and/or R^{4c} is/are preferably different from hydrogen. Particular preference is given to compounds (I) wherein R^{4a} and/or R^{4c} is/are selected from the group consisting of halogen, C₁-C₆-alkyl, C₁-C₆-haloalkyl, phenyl and benzyl which may be unsubstituted or may carry any combination of 1, 2, 3, 4 or 5, preferably 1, 2 or 3 radicals R^{b4}. In this particular embodiment, R^{4b} and R^{4d} and optionally one of R^{4a} and R^{4c} are preferably hydrogen or C₁-C₆-alkyl and more preferably hydrogen.

In another preferred embodiment of the invention, the radicals R^{2a} and R^{4a} together form a bridging bivalent radical. Preferred examples of such bridging radicals are C(O)—CH₂, C(S)—CH₂, CH₂—CH₂, S(O)₂—CH₂, S(O)—CH₂, C(O)—O, C(S)—O, S(O)₂—O, S(O)—O, C(O)—NH, C(S)—NH, S(O)₂—NH, S(O)—NH, wherein the last eight mentioned radicals are attached to the nitrogen atom via the carbonyl group, the thiocarbonyl group or the sulphur atom respectively. More preferred examples of such radicals are C(O)—CH₂, C(S)—CH₂, CH₂—CH₂, S(O)₂—CH₂, S(O)—CH₂.

Likewise preferred are compounds of general formula (I), wherein R^{4a} together with R^{4b} and/or R^{4c} together with R^{4d} form a radical ═O, ═NR^{c} or ═CR^{d}R^{e}. Preferably, the radicals R^{c}, R^{d} and R^{e} are selected from the group consisting of hydrogen, C₁-C₆-alkyl, phenyl, hydroxy, C₁-C₆-alkoxy, C₁-C₆-alkylamino and di-(C₁-C₆-alkyl)amino.

R^{b4} is preferably selected from halogen, hydroxy, amino, C₁-C₆-alkyl, C₁-C₆-haloalkyl, C₁-C₆-alkoxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylcarbonyl, C₁-C₆-alkoxycarbonyl and C₁-C₆-alkylcarbonyloxy and more preferably from halogen, C₁-C₆-alkyl, C₁-C₆-haloalkyl, C₁-C₆-alkoxy and C₁-C₆-haloalkoxy.

Specifically, all radicals R^{4a}, R^{4b}, R^{4c} and R^{4d} are hydrogen.

Preferably, each R²¹ is independently selected from the group consisting of halogen, OH, SH, C₁-C₆-alkyl, C₁-C₆-haloalkyl, C₁-C₆-alkoxy, C₁-C₆-haloalkoxy, C₁-C₆-alkylthio and C₁-C₆-haloalkylthio, more preferably from halogen, C₁-C₆-alkyl, C₁-C₆-haloalkyl, C₁-C₆-alkoxy, C₁-C₆-haloalkoxy, C₁-C₆-alkylthio and C₁-C₆-haloalkylthio and specifically from halogen C₁-C₆-alkoxy and C₁-C₆-haloalkoxy.

In one preferred embodiment, n is 0 or 1.

In an alternatively preferred embodiment, n is 2.

In one preferred embodiment of the present invention the variable X in compounds of the general formula (I) is sulfur. In another preferred embodiment X is O. In yet another preferred embodiment X is NR⁵. Particularly preferably, X is S.

In one preferred embodiment of the present invention the variable Y in compounds of the general formula (I) is sulfur. In another preferred embodiment Y is O. In yet another preferred embodiment Y is NR^{5a}. Particularly preferably, Y is S.

R⁵ and R^{5a} are preferably selected from hydrogen, C₁-C₆-alkyl, formyl, CN, C(O)NR^{a}R^{b}, C(S)NR^{a}R^{b}, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl, C₁-C₆-alkylthiocarbonyl, benzoyl, and hetarylcarbonyl, wherein each of the 2 last mentioned radicals may be unsubstituted or may carry any combination of 1, 2 or 3 radicals R^{b5}, and wherein the heteroaromatic ring in hetarylcarbonyl is 5- or 6-membered and contains 1, 2, 3 or 4, preferably 1, 2 or 3 heteroatoms selected from oxygen, sulfur and nitrogen as ring members. More preferably, R⁵ and R^{5a} are hydrogen or C₁-C₆-alkyl.

R^{b5} is preferably selected from halogen, hydroxy, amino, C₁-C₆-alkyl, C₁-C₆-haloalkyl, C₁-C₆-alkoxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylcarbonyl, C₁-C₆-alkoxycarbonyl and C₁-C₆-alkylcarbonyloxy and more preferably from halogen, C₁-C₆-alkyl, C₁-C₆-haloalkyl, C₁-C₆-alkoxy and C₁-C₆-haloalkoxy.

T is preferably selected from —OR^{T1} and —O—C(O)—R^{T3}.

R^{T1} is preferably selected from hydrogen and C₁-C₆-alkyl. More preferably, R^{T1} is hydrogen.

R^{T3} is preferably selected from C₁-C₆-alkyl which may carry 1, 2 or 3 substituents R^{Ta}; C₃-C₆-cycloalkyl which may carry 1, 2 or 3 substituents R^{Tb}; phenyl which may carry 1, 2 or 3 substituents R^{Tc}; naphthyl which may carry 1, 2 or 3 substituents R^{Tc}; benzo-1,3-dioxolanyl which may carry 1, 2 or 3 substituents R^{Tc}; and 5- or 6-membered hetaryl containing 1, 2 or 3 heteroatoms selected from O, S and N as ring members, which may carry 1, 2 or 3 substituents R^{Td}.

Preferably, each R^{Ta} is independently selected from the group consisting of halogen, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, and 5- or 6-membered hetaryl, where the phenyl moiety and the hetaryl ring in the 3 last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy. More preferably, each R^{Ta} is independently selected from the group consisting of C₁-C₄-alkoxy, C₁-C₄-alkylthio, C₁-C₄-alkylcarbonyl, C₁-C₄-alkoxycarbonyl, phenyl, phenyloxy, and 5- or 6-membered hetaryl.

Preferably, each R^{Tc} is independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, and 5- or 6-membered hetaryl, where the phenyl moiety and the hetaryl ring in the 3 last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy.

Preferably, each R^{Tc} is independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, and C₁-C₄-haloalkylcarbonyloxy and 5- or 6-membered hetaryl, where the hetaryl ring may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy.

Preferably, each R^{Td} is independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, and 5- or 6-membered hetaryl, where the phenyl moiety and the hetaryl ring in the 3 last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy. More preferably, each R^{Td} is independently selected from the group consisting of halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy and phenyl, where the phenyl moiety may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy.

In one preferred embodiment, Z is a group of formula (II.A) or (II.B). Preferred meanings of the variables in theses groups are listed above.

Compounds (I) wherein Z is a group of formula (II.A) or (II.B) represent tautomers in case R^{2a} and R^{2b} are hydrogen.

In an alternatively preferred embodiment, Z is a group of formula (II.C). Preferred meanings of the variables in theses groups are listed above.

Compounds (I) wherein Z is a group of formula (II.C) can serve as intermediates in the preparation of compounds (I) wherein Z is a group of formula (II.A) or (II.B). For example, in compounds (I) wherein T is a group —O—R^{T1}, the group T can be converted into a leaving group, especially if R^{T1} is H, e.g. by converting it into the corresponding tosylate or halogenide. Compounds (I) wherein T is a group —O—C(O)—R^{T3} can be reacted directly to the corresponding compound (I) wherein Z is a group (II.A) or (II.B). However, compounds (I) wherein Z is a group of formula (II.C) have themselves a good activity profile against harmful animal pests and can thus be used without further conversion.

Particularly preferred compounds (I) are those of the formulae (I.A.1), (I.A.2), (I.B.1), (I.B.2), (I.C.1), (I.C.2), (I.C.3) and (I.C.4) below:

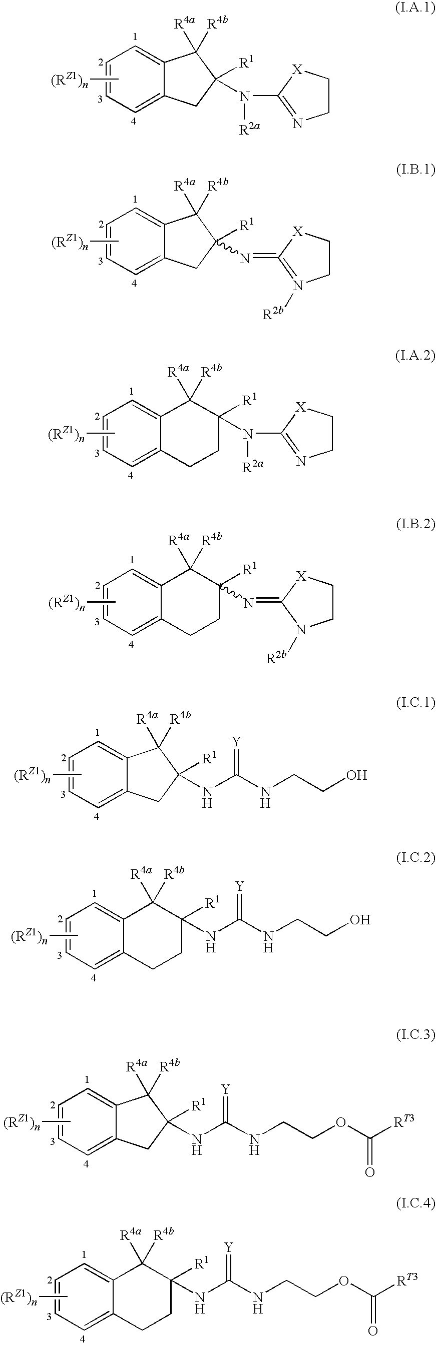

in which the variables R¹, R^{2a}, R^{2b}, R^{4a}, R^{4b}, R^{Z1}, R^{T3}, X, Y and n have the general or preferred meanings given above.

Examples of particularly preferred compounds of the general formula I are the compounds I compiled in Tables 1 to 176 below. Moreover, the meanings mentioned for the individual variables in the tables are per se, independently of the combination in which they are mentioned, a particularly preferred embodiment of the substituents in question.

### Table 1

Compounds of the formulae (I.A.1) or (I.B.1) and their mixtures, wherein X is O, R¹ is hydrogen, R^{2a} or R^{2b} is hydrogen, and the combination of (R^{Z1})ₙ, R^{4a} and R^{4b} for a compound corresponds in each case to one row of table A.

### Table 2

Compounds of the formulae (I.A.1) or (I.B.1) and their mixtures, wherein X is S, R¹ is hydrogen, R^{2a} or R^{2b} is hydrogen, and the combination of (R^{Z1})ₙ, R^{4a} and R^{4b} for a compound corresponds in each case to one row of table A.

### Table 3

Compounds of the formulae (I.A.1) or (I.B.1) and their mixtures, wherein X is NH, R¹ is hydrogen, R^{2a} or R^{2b} is hydrogen, and the combination of (R^{Z1})ₙ, R^{4a} and R^{4b} for a compound corresponds in each case to one row of table A.

### Table 4

Compounds of the formulae (I.A.1) or (I.B.1) and their mixtures, wherein X is NCH₃, R¹ is hydrogen, R^{2a} or R^{2b} is hydrogen, and the combination of (R^{Z1})ₙ, R^{4a} and R^{4b} for a compound corresponds in each case to one row of table A.

### Table 5

Compounds of the formulae (I.A.1) or (I.B.1) and their mixtures, wherein X is O, R¹ is CH₃, R^{2a} or R^{2b} is hydrogen, and the combination of (R^{Z1})ₙ, R^{4a} and R^{4b} for a compound corresponds in each case to one row of table A.

### Table 6

Compounds of the formulae (I.A.1) or (I.B.1) and their mixtures, wherein X is S, R¹ is CH₃, R^{2a} or R^{2b} is hydrogen, and the combination of (R^{Z1})ₙ, R^{4a} and R^{4b} for a compound corresponds in each case to one row of table A.

### Table 7

Compounds of the formulae (I.A.1) or (I.B.1) and their mixtures, wherein X is NH, R¹ is CH₃, R^{2a} or R^{2b} is hydrogen, and the combination of (R^{Z1})ₙ, R^{4a} and R^{4b} for a compound corresponds in each case to one row of table A.

### Table 8

Compounds of the formulae (I.A.1) or (I.B.1) and their mixtures, wherein X is NCH₃, R¹ is CH₃, R^{2a} or R^{2b} is hydrogen, and the combination 1\ₙ nation of (R^{Z1})ₙ, R^{4a} and R^{4b} for a compound corresponds in each case to one row of table A.

### Table 9

Compounds of the formulae (I.A.1) or (I.B.1) and their mixtures, wherein X is O, R¹ is phenyl, R^{2a} or R^{2b} is hydrogen, and the combination of (R^{Z1})ₙ, R^{4a} and R^{4b} for a compound corresponds in each case to one row of table A.

### Table 10

Compounds of the formulae (I.A.1) or (I.B.1) and their mixtures, wherein X is S, R¹ is phenyl, R^{2a} or R^{2b} is hydrogen, and the combination of (R^{Z1})ₙ, R^{4a} and R^{4b} for a compound corresponds in each case to one row of table A.

### Table 11

Compounds of the formulae (I.A.1) or (I.B.1) and their mixtures, wherein X is NH, R¹ is phenyl, R^{2a} or R^{2b} is hydrogen, and the combination of (R^{Z1})ₙ, R^{4a} and R^{4b} for a compound corresponds in each case to one row of table A.

### Table 12

Compounds of the formulae (I.A.1) or (I.B.1) and their mixtures, wherein X is NCH₃, R¹ is phenyl, R^{2a} or R^{2b} is hydrogen, and the combination of (R^{Z1})ₙ, R^{4a} and R^{4b} for a compound corresponds in each case to one row of table A.

### Table 13

Compounds of the formulae (I.A.1) or (I.B.1) and their mixtures, wherein X is O, R¹ is benzyl, R^{2a} or R^{2b} is hydrogen, and the combination of (R^{Z1})ₙ, R^{4a} and R^{4b} for a compound pound corresponds in each case to one row of table A.

### Table 14

Compounds of the formulae (I.A.1) or (I.B.1) and their mixtures, wherein X is S, R¹ is benzyl, R^{2a} or R^{2b} is hydrogen, and the combination of (R^{Z1})ₙ, R^{4a} and R^{4b} for a compound corresponds in each case to one row of table A.

### Table 15

Compounds of the formulae (I.A.1) or (I.B.1) and their mixtures, wherein X is NH, R¹ is benzyl, R^{2a} or R^{2b} is hydrogen, and the combination of (R^{Z1})ₙ, R^{4a} and R^{4b} for a compound corresponds in each case to one row of table A.

### Table 16

Compounds of the formulae (I.A.1) or (I.B.1) and their mixtures, wherein X is NCH₃, R¹ is benzyl, R^{2a} or R^{2b} is hydrogen, and the combination of (R^{Z1})ₙ, R^{4a} and R^{4b} for a compound corresponds in each case to one row of table A.

### Tables 17 to 32

Compounds of the formulae (I.A.1) or (I.B.1) and their mixtures, wherein the combination of X and R¹ is as defined in any of Tables 1 to 16, the combination of (R^{Z1})ₙ, R^{4a} and R^{4b} for a compound corresponds in each case to one row of Table A and R^{2a} or R^{2b} is CH₃ instead of hydrogen.

### Tables 33 to 48

Compounds of the formulae (I.A.1) or (I.B.1) and their mixtures, wherein the combination of X and R¹ is as defined in any of Tables 1 to 16, the combination of (R^{Z1})ₙ, R^{4a} and R^{4b} for a compound corresponds in each case to one row of Table A and R^{2a} or R^{2b} is —C(O)CH₃ instead of hydrogen.

### Tables 49 to 64

Compounds of the formulae (I.A.1) or (I.B.1) and their mixtures, wherein the combination of X and R¹ is as defined in any of Tables 1 to 16, the combination of (R^{Z1})ₙ, R^{4a} and R^{4b} for a compound corresponds in each case to one row of Table A and R^{2a} or R^{2b} is benzoyl instead of hydrogen.

### Tables 65 to 80

Compounds of the formulae (I.A.1) or (I.B.1) and their mixtures, wherein the combination of X and R¹ is as defined in any of Tables 1 to 16, the combination of (R^{Z1})ₙ, R^{4a} and R^{4b} for a compound corresponds in each case to one row of Table A and R^{2a} or R^{2b} is thiazolin-2-yl instead of hydrogen.

### Tables 81 to 96

Compounds of the formulae (I.A.1) or (I.B.1) and their mixtures, wherein the combination of X and R¹ is as defined in any of Tables 1 to 16, the combination of (R^{Z1})ₙ, R^{4a} and R^{4b} for a compound corresponds in each case to one row of Table A and R^{2a} or R^{2b} is oxazolin-2-yl instead of hydrogen.

### Tables 97 to 112

Compounds of the formulae (I.A.1) or (I.B.1) and their mixtures, wherein the combination of X and R¹ is as defined in any of Tables 1 to 16, the combination of (R^{Z1})ₙ, R^{4a} and R^{4b} for a compound corresponds in each case to one row of Table A and R^{2a} or R^{2b} is imidazolin-2-yl instead of hydrogen.

### Table 113

Compounds of the formula (I.C.1), wherein Y is O, R¹ is hydrogen and the combination of (R^{Z1})ⁿ, R^{4a} and R^{4b} for a compound corresponds in each case to one row of table A.

### Table 114

Compounds of the formula (I.C.1), wherein Y is S, R¹ is hydrogen and the combination of (R^{Z1})ₙ, R^{4a} and R^{4b} for a compound corresponds in each case to one row of table A.

### Table 115

Compounds of the formula (I.C.1), wherein Y is NH, R¹ is hydrogen and the combination of (R^{Z1})ₙ, R^{4a} and R^{4b} for a compound corresponds in each case to one row of table A.

### Table 116

Compounds of the formula (I.C.1), wherein Y is NCH₃, R¹ is hydrogen and the combination of (R^{Z1})ₙ, R^{4a} and R^{4b} for a compound corresponds in each case to one row of table A.

### Table 117

Compounds of the formula (I.C.1), wherein Y is O, R¹ is CH₃ and the combination of (R^{Z1})ₙ, R^{4a} and R^{4b} for a compound corresponds in each case to one row of table A.

### Table 118

Compounds of the formula (I.C.1), wherein Y is S, R¹ is CH₃ and the combination of (R^{Z1})ₙ, R^{4a} and R^{4b} for a compound corresponds in each case to one row of table A.

### Table 119

Compounds of the formula (I.C.1), wherein Y is NH, R¹ is CH₃ and the combination of (R^{Z1})ₙ, R^{4a} and R^{4b} for a compound corresponds in each case to one row of table A.

### Table 120

Compounds of the formula (I.C.1), wherein Y is NCH₃, R¹ is CH₃ and the combination of (R^{Z1})ₙ, R^{4a} and R^{4b} for a compound corresponds in each case to one row of table A.

### Table 121

Compounds of the formula (I.C.1), wherein Y is O, R¹ is phenyl and the combination of (R^{Z1})ₙ, R^{4a} and R^{4b} for a compound corresponds in each case to one row of table A.

### Table 122

Compounds of the formula (I.C.1), wherein Y is S, R¹ is phenyl and the combination of (R^{Z1})ₙ, R^{4a} and R^{4b} for a compound corresponds in each case to one row of table A.

### Table 123

Compounds of the formula (I.C.1), wherein Y is NH, R¹ is phenyl and the combination of (R^{Z1})ₙ, R^{4a} and R^{4b} for a compound corresponds in each case to one row of table A.

### Table 124

Compounds of the formula (I.C.1), wherein Y is NCH₃, R¹ is phenyl and the combination of (R^{Z1})ₙ, R^{4a} and R^{4b} for a compound corresponds in each case to one row of table A.

### Table 125

Compounds of the formula (I.C.1), wherein Y is O, R¹ is benzyl and the combination of (R^{Z1})ₙ, R^{4a} and R^{4b} for a compound corresponds in each case to one row of table A.

### Table 126

Compounds of the formula (I.C.1), wherein Y is S, R¹ is benzyl and the combination of (R^{Z1})ₙ, R^{4a} and R^{4b} for a compound corresponds in each case to one row of table A.

### Table 127

Compounds of the formula (I.C.1), wherein Y is NH, R¹ is benzyl and the combination of (R^{Z1})ₙ, R^{4a} and R^{4b} for a compound corresponds in each case to one row of table A.

### Table 128

Compounds of the formula (I.C.1), wherein Y is NCH₃, R¹ is benzyl and the combination of (R^{Z1})ₙ, R^{4a} and R^{4b} for a compound corresponds in each case to one row of table A.

### Table 129

Compounds of the formula (I.C.2), wherein Y is O, R¹ is hydrogen and the combination of (R^{Z1})ₙ, R^{4a} and R^{4b} for a compound corresponds in each case to one row of table A.

### Table 130

Compounds of the formula (I.C.2), wherein Y is S, R¹ is hydrogen and the combination of (R^{Z1})ₙ, R^{4a} and R^{4b} for a compound corresponds in each case to one row of table A.

### Table 131

Compounds of the formula (I.C.2), wherein Y is NH, R¹ is hydrogen and the combination of (R^{Z1})ₙ, R^{4a} and R^{4b} for a compound corresponds in each case to one row of table A.

### Table 132

Compounds of the formula (I.C.2), wherein Y is NCH₃, R¹ is hydrogen and the combination of (R^{Z1})ₙ, R^{4a} and R^{4b} for a compound corresponds in each case to one row of table A.

### Table 133

Compounds of the formula (I.C.2), wherein Y is O, R¹ is CH₃ and the combination of (R^{Z1})ₙ, R^{4a} and R^{4b} for a compound corresponds in each case to one row of table A.

### Table 134

Compounds of the formula (I.C.2), wherein Y is S, R¹ is CH₃ and the combination of (R^{Z1})ₙ, R^{4a} and R^{4b} for a compound corresponds in each case to one row of table A.

### Table 135

Compounds of the formula (I.C.2), wherein Y is NH, R¹ is CH₃ and the combination of (R^{Z1})ₙ, R^{4a} and R^{4b} for a compound corresponds in each case to one row of table A.

### Table 136

Compounds of the formula (I.C.2), wherein Y is NCH₃, R¹ is CH₃ and the combination of (R^{Z1})ₙ, R^{4a} and R^{4b} for a compound corresponds in each case to one row of table A.

### Table 137

Compounds of the formula (I.C.2), wherein Y is O, R¹ is phenyl and the combination of (R^{Z1})ₙ, R^{4a} and R^{4b} for a compound corresponds in each case to one row of table A.

### Table 138

Compounds of the formula (I.C.2), wherein Y is S, R¹ is phenyl and the combination of) R^{4a} and R^{4b} for a compound corresponds in each case to one row of table A.

### Table 139

Compounds of the formula (I.C.2), wherein Y is NH, R¹ is phenyl and the combination of (R^{Z1})ₙ, R^{4a} and R^{4b} for a compound corresponds in each case to one row of table A.

### Table 140

Compounds of the formula (I.C.2), wherein Y is NCH₃, R¹ is phenyl and the combination of (R^{Z1})ⁿ, R^{4a} and R^{4b} for a compound corresponds in each case to one row of table A.

### Table 141

Compounds of the formula (I.C.2), wherein Y is O, R¹ is benzyl and the combination of (R^{Z1})ₙ, R^{4a} and R^{4b} for a compound corresponds in each case to one row of table A.

### Table 142

Compounds of the formula (I.C.2), wherein Y is S, R¹ is benzyl and the combination of (R^{Z1})ₙ, R^{4a} and R^{4b} for a compound corresponds in each case to one row of table A.

### Table 143

Compounds of the formula (I.C.2), wherein Y is NH, R¹ is benzyl and the combination of (R^{Z1})ₙ, R^{4a} and R^{4b} for a compound corresponds in each case to one row of table A.

### Table 144

Compounds of the formula (I.C.2), wherein Y is NCH₃, R¹ is benzyl and the combination of (R^{Z1})ₙ, R^{4a} and R^{4b} for a compound corresponds in each case to one row of table A.

### Table 145

Compounds of the formula (I.C.3), wherein Y is O, R¹ is hydrogen, R^{T3} has in each case one of the meanings given in one row of table B and the combination of (R^{Z1})ₙ, R^{4a} and R^{4b} for a compound corresponds in each case to one row of table A.

### Table 146

Compounds of the formula (I.C.3), wherein Y is S, R¹ is hydrogen, R^{T3} has in each case one of the meanings given in one row of table B and the combination of (R^{Z1})ₙ, R^{4a} and R^{4b} for a compound corresponds in each case to one row of table A.

### Table 147

Compounds of the formula (I.C.3), wherein Y is NH, R¹ is hydrogen, R^{T3} has in each case one of the meanings given in one row of table B and the combination of (R^{Z1})ₙ, R^{4a} and R^{4b} for a compound corresponds in each case to one row of table A.

### Table 148

Compounds of the formula (I.C.3), wherein Y is NCH₃, R¹ is hydrogen, R^{T3} has in each case one of the meanings given in one row of table B and the combination of (R^{Z1})ₙ, R^{4a} and R^{4b} for a compound corresponds in each case to one row of table A.

### Table 149

Compounds of the formula (I.C.3), wherein Y is O, R¹ is CH₃, R^{T3} has in each case one of the meanings given in one row of table B and the combination of (R^{Z1})ₙ, R^{4a} and R^{4b} for a compound corresponds in each case to one row of table A.

### Table 150

Compounds of the formula (I.C.3), wherein Y is S, R¹ is CH₃, R^{T3} has in each case one of the meanings given in one row of table B and the combination of (R^{Z1})ₙ, R^{4a} and R^{4b} for a compound corresponds in each case to one row of table A.

### Table 151

Compounds of the formula (I.C.3), wherein Y is NH, R¹ is CH₃, R^{T3} has in each case one of the meanings given in one row of table B and the combination of (R^{Z1})ₙ, R^{4a} and R^{4b} for a compound corresponds in each case to one row of table A.

### Table 152

Compounds of the formula (I.C.3), wherein Y is NCH₃, R¹ is CH₃, R^{T3} has in each case one of the meanings given in one row of table B and the combination of (R^{Z1})ₙ, R^{4a} and R^{4b} for a compound corresponds in each case to one row of table A.

### Table 153

Compounds of the formula (I.C.3), wherein Y is O, R¹ is phenyl, R^{T3} has in each case one of the meanings given in one row of table B and the combination of (R^{Z1})ₙ, R^{4a} and R^{4b} for a compound corresponds in each case to one row of table A.

### Table 154

Compounds of the formula (I.C.3), wherein Y is S, R¹ is phenyl, R^{T3} has in each case one of the meanings given in one row of table B and the combination of (R^{Z1})ₙ, R^{4a} and R^{4b} for a compound corresponds in each case to one row of table A.

### Table 155

Compounds of the formula (I.C.3), wherein Y is NH, R¹ is phenyl, R^{T3} has in each case one of the meanings given in one row of table B and the combination of (R^{Z1})ₙ, R^{4a} and R^{4b} for a compound corresponds in each case to one row of table A.

### Table 156

Compounds of the formula (I.C.3), wherein Y is NCH₃, R¹ is phenyl, R^{T3} has in each case one of the meanings given in one row of table B and the combination of (R^{Z1})ₙ, R^{4a} and R^{4b} for a compound corresponds in each case to one row of table A.

### Table 157

Compounds of the formula (I.C.3), wherein Y is O, R¹ is benzyl, R^{T3} has in each case one of the meanings given in one row of table B and the combination of (R^{Z1})ₙ, R^{4a} and R^{4b} for a compound corresponds in each case to one row of table A.

### Table 158

Compounds of the formula (I.C.3), wherein Y is S, R¹ is benzyl, R^{T3} has in each case one of the meanings given in one row of table B and the combination of (R^{Z1})ₙ, R^{4a} and R^{4b} for a compound corresponds in each case to one row of table A.

### Table 159

Compounds of the formula (I.C.3), wherein Y is NH, R¹ is benzyl, R^{T3} has in each case one of the meanings given in one row of table B and the combination of (R^{Z1})ₙ, R^{4a} and R^{4b} for a compound corresponds in each case to one row of table A.

### Table 160

Compounds of the formula (I.C.3), wherein Y is NCH₃, R¹ is benzyl, R^{T3} has in each case one of the meanings given in one row of table B and the combination of (R^{Z1})ₙ, R^{4a} and R^{4b} for a compound corresponds in each case to one row of table A.

### Table 161

Compounds of the formula (I.C.4), wherein Y is O, R¹ is hydrogen, R^{T3} has in each case one of the meanings given in one row of table B and the combination of (R^{Z1})ₙ, R^{4a} and R^{4b} for a compound corresponds in each case to one row of table A.

### Table 162

Compounds of the formula (I.C.4), wherein Y is S, R¹ is hydrogen, R^{T3} has in each case one of the meanings given in one row of table B and the combination of (R^{Z1})ₙ, R^{4a} and R^{4b} for a compound corresponds in each case to one row of table A.

### Table 163

Compounds of the formula (I.C.4), wherein Y is NH, R¹ is hydrogen, R^{T3} has in each case one of the meanings given in one row of table B and the combination of (R^{Z1})ₙ, R^{4a} and R^{4b} for a compound corresponds in each case to one row of table A.

### Table 164

Compounds of the formula (I.C.4), wherein Y is NCH₃, R¹ is hydrogen, R^{T3} has in each case one of the meanings given in one row of table B and the combination of (R^{Z1})ₙ, R^{4a} and R^{4b} for a compound corresponds in each case to one row of table A.

### Table 165

Compounds of the formula (I.C.4), wherein Y is O, R¹ is CH₃, R^{T3} has in each case one of the meanings given in one row of table B and the combination of (R^{Z1})ₙ, R^{4a} and R^{4b} for a compound corresponds in each case to one row of table A.

### Table 166

Compounds of the formula (I.C.4), wherein Y is S, R¹ is CH₃, R^{T3} has in each case one of the meanings given in one row of table B and the combination of (R^{Z1})ₙ, R^{4a} and R^{4b} for a compound corresponds in each case to one row of table A.

### Table 167

Compounds of the formula (I.C.4), wherein Y is NH, R¹ is CH₃, R^{T3} has in each case one of the meanings given in one row of table B and the combination of (R^{Z1})ₙ, R^{4a} and R^{4b} for a compound corresponds in each case to one row of table A.

### Table 168

Compounds of the formula (I.C.4), wherein Y is NCH₃, R¹ is CH₃, R^{T3} has in each case one of the meanings given in one row of table B and the combination of (R^{Z1})ₙ, R^{4a} and R^{4b} for a compound corresponds in each case to one row of table A.

### Table 169

Compounds of the formula (I.C.4), wherein Y is O, R¹ is phenyl, R^{T3} has in each case one of the meanings given in one row of table B and the combination of (R^{Z1})ₙ, R^{4a} and R^{4b} for a compound corresponds in each case to one row of table A.

### Table 170

Compounds of the formula (I.C.4), wherein Y is S, R¹ is phenyl, R^{T3} has in each case one of the meanings given in one row of table B and the combination of (R^{Z1})ₙ, R^{4a} and R^{4b} for a compound corresponds in each case to one row of table A.

### Table 171

Compounds of the formula (I.C.4), wherein Y is NH, R¹ is phenyl, R^{T3} has in each case one of the meanings given in one row of table B and the combination of (R^{Z1})ₙ, R^{4a} and R^{4b} for a compound corresponds in each case to one row of table A.

### Table 172

Compounds of the formula (I.C.4), wherein Y is NCH₃, R¹ is phenyl, R^{T3} has in each case one of the meanings given in one row of table B and the combination of (R^{Z1})ₙ, R^{4a} and R^{4b} for a compound corresponds in each case to one row of table A.

### Table 173

Compounds of the formula (I.C.4), wherein Y is O, R¹ is benzyl, R^{T3} has in each case one of the meanings given in one row of table B and the combination of (R^{Z1})ₙ, R^{4a} and R^{4b} for a compound corresponds in each case to one row of table A.

### Table 174

Compounds of the formula (I.C.4), wherein Y is S, R¹ is benzyl, R^{T3} has in each case one of the meanings given in one row of table B and the combination of (R^{Z1})ₙ, R^{4a} and R^{4b} for a compound corresponds in each case to one row of table A.

### Table 175

Compounds of the formula (I.C.4), wherein Y is NH, R¹ is benzyl, R^{T3} has in each case one of the meanings given in one row of table B and the combination of (R^{Z1})ₙ, R^{4a} and R^{4b} for a compound corresponds in each case to one row of table A.

### Table 176

Compounds of the formula (I.C.4), wherein Y is NCH₃, R¹ is benzyl, R^{T3} has in each case one of the meanings given in one row of table B and the combination of (R^{Z1})ₙ, R^{4a} and R^{4b} for a compound corresponds in each case to one row of table A.

TABLE A

| | (R^{Z1})ₙ | R^{4a} | R^{4b} |
|---|---|---|---|
| A-1 | 1-CH₃ | H | H |
| A-2 | 2-CH₃ | H | H |
| A-3 | 3-CH₃ | H | H |
| A-4 | 4-CH₃ | H | H |
| A-5 | 1-CH₃, 3-CH₃ | H | H |
| A-6 | 1-CH₃, 3-CH₃, 4-CH₃ | H | H |
| A-7 | 3-CH₃, 4-CH₃ | H | H |
| A-8 | 1-Cl | H | H |
| A-9 | 2-Cl | H | H |
| A-10 | 3-Cl | H | H |
| A-11 | 4-Cl | H | H |
| A-12 | 1-Cl, 3-Cl | H | H |
| A-13 | 1-Cl, 3-Cl, 4-Cl | H | H |
| A-14 | 3-Cl, 4-Cl | H | H |
| A-15 | 1-F | H | H |
| A-16 | 2-F | H | H |
| A-17 | 3-F | H | H |
| A-18 | 4-F | H | H |
| A-19 | 1-F, 3-F | H | H |
| A-20 | 1-F, 3-F, 4-F | H | H |
| A-21 | 3-F, 4-F | H | H |
| A-22 | 1-Br | H | H |
| A-23 | 2-Br | H | H |
| A-24 | 3-Br | H | H |
| A-25 | 4-Br | H | H |
| A-26 | 1-Br, 3-Br | H | H |
| A-27 | 1-Br, 3-Br, 4-Br | H | H |
| A-28 | 3-Br, 4-Br | H | H |
| A-29 | 1-CF₃ | H | H |
| A-30 | 2-CF₃ | H | H |
| A-31 | 3-CF₃ | H | H |
| A-32 | 4-CF₃ | H | H |
| A-33 | 1CF₃, 3-CF₃ | H | H |
| A-34 | 1-CF₃, 3-CF₃, 4-CF₃ | H | H |
| A-35 | 3-CF₃, 4-CF3 | H | H |
| A-36 | 1-OCH₃ | H | H |
| A-37 | 2-OCH₃ | H | H |
| A-38 | 3-OCH₃ | H | H |
| A-39 | 4-OCH₃ | H | H |
| A-40 | 1-OCH₃, 3-OCH₃ | H | H |
| A-41 | 1-OCH₃, 3-OCH₃, 4-OCH₃ | H | H |
| A-42 | 3,4-OCH₃, OCH₃ | H | H |
| A-43 | 1-CH₃, 3-Cl | H | H |
| A-44 | 1-Cl, 3-CH₃ | H | H |
| A-45 | 1-CH₃, 3-F | H | H |
| A-46 | 1-F, 3-CH₃ | H | H |
| A-47 | 1-CH₃, 3-Br | H | H |
| A-48 | 1-Br, 3-CH₃ | H | H |
| A-49 | 1-CH₃, 3-CF₃ | H | H |
| A-50 | 1-CF₃, 3-CH₃ | H | H |
| A-51 | 1-CH₃, 3 OCH₃ | H | H |
| A-52 | 1-OCH₃, 3-CH₃ | H | H |
| A-53 | 1-Cl, 3-F | H | H |
| A-54 | 1-F, 3-Cl | H | H |
| A-55 | 1-Cl, 3-Br | H | H |
| A-56 | 1-Br, 3-Cl | H | H |
| A-57 | 1-Cl, 3-CF3 | H | H |
| A-58 | 11-CF₃, 3-Cl | H | H |
| A-59 | 1-Cl, 3-OCH₃ | H | H |
| A-60 | 1-OCH₃, 3-Cl | H | H |
| A-61 | 1-F, 3-Br | H | H |
| A-62 | 1-Br, 3-F | H | H |
| A-63 | 1-F, 3-CF₃ | H | H |
| A-64 | 1-CF₃, 3-F | H | H |
| A-65 | 1-F, 3-OCH₃ | H | H |
| A-66 | 1-OCH₃, 3-F | H | H |
| A-67 | 1-Br, 3-CF3 | H | H |
| A-68 | 1-CF₃, 3-Br | H | H |
| A-69 | 1-Br, 3-OCH₃ | H | H |
| A-70 | 1-OCH₃, 3-Br | H | H |
| A-71 | 1-CF₃, 3-OCH₃ | H | H |
| A-72 | 1-OCH₃, 3-CF₃ | H | H |
| A-73 | 1-CH₃ | CH₃ | H |
| A-74 | 2-CH₃ | CH₃ | H |
| A-75 | 3-CH₃ | CH₃ | H |
| A-76 | 4-CH₃ | CH₃ | H |
| A-77 | 1-CH₃, 3-CH₃ | CH₃ | H |
| A-78 | 1-CH₃, 3-CH₃, 4-CH₃ | CH₃ | H |
| A-79 | 3-CH₃, 4-CH₃ | CH₃ | H |
| A-80 | 1-Cl | CH₃ | H |
| A-81 | 2-Cl | CH₃ | H |
| A-82 | 3-Cl | CH₃ | H |
| A-83 | 4-Cl | CH₃ | H |
| A-84 | 1-Cl, 3-Cl | CH₃ | H |
| A-85 | 1-Cl, 3-Cl, 4-Cl | CH₃ | H |
| A-86 | 3-Cl, 4-Cl | CH₃ | H |
| A-87 | 1-F | CH₃ | H |
| A-88 | 2-F | CH₃ | H |
| A-89 | 3-F | CH₃ | H |
| A-90 | 4-F | CH₃ | H |
| A-91 | 1-F, 3-F | CH₃ | H |
| A-92 | 1-F, 3-F, 4-F | CH₃ | H |
| A-93 | 3-F, 4-F | CH₃ | H |
| A-94 | 1-Br | CH₃ | H |
| A-95 | 2-Br | CH₃ | H |
| A-96 | 3-Br | CH₃ | H |
| A-97 | 4-Br | CH₃ | H |
| A-98 | 1-Br, 3-Br | CH₃ | H |
| A-99 | 1-Br, 3-Br, 4-Br | CH₃ | H |
| A-100 | 3-Br, 4-Br | CH₃ | H |
| A-101 | 1-CF₃ | CH₃ | H |
| A-102 | 2-CF₃ | CH₃ | H |
| A-103 | 3-CF₃ | CH₃ | H |
| A-104 | 4-CF₃ | CH₃ | H |
| A-105 | 1CF₃, 3-CF₃ | CH₃ | H |
| A-106 | 1-CF₃, 3-CF₃, 4-CF₃ | CH₃ | H |
| A-107 | 3-CF₃, 4-CF3 | CH₃ | H |
| A-108 | 1-OCH₃ | CH₃ | H |
| A-109 | 2-OCH₃ | CH₃ | H |
| A-110 | 3-OCH₃ | CH₃ | H |
| A-111 | 4-OCH₃ | CH₃ | H |
| A-112 | 1-OCH₃, 3-OCH₃ | CH₃ | H |
| A-113 | 1-OCH₃, 3-OCH₃, 4-OCH₃ | CH₃ | H |
| A-114 | 3,4-OCH₃, OCH₃ | CH₃ | H |
| A-115 | 1-CH₃, 3-Cl | CH₃ | H |
| A-116 | 1-Cl, 3-CH₃ | CH₃ | H |
| A-117 | 1-CH₃, 3-F | CH₃ | H |
| A-118 | 1-F, 3-CH₃ | CH₃ | H |
| A-119 | 1-CH₃, 3-Br | CH₃ | H |
| A-120 | 1-Br, 3-CH₃ | CH₃ | H |
| A-121 | 1-CH₃, 3-CF₃ | CH₃ | H |
| A-122 | 1-CF₃, 3-CH₃ | CH₃ | H |
| A-123 | 1-CH₃, 3 OCH₃ | CH₃ | H |
| A-124 | 1-OCH₃, 3-CH₃ | CH₃ | H |
| A-125 | 1-Cl, 3-F | CH₃ | H |
| A-126 | 1-F, 3-Cl | CH₃ | H |
| A-127 | 1-Cl, 3-Br | CH₃ | H |
| A-128 | 1-Br, 3-Cl | CH₃ | H |
| A-129 | 1-Cl, 3-CF3 | CH₃ | H |
| A-130 | 11-CF₃, 3-Cl | CH₃ | H |
| A-131 | 1-Cl, 3-OCH₃ | CH₃ | H |
| A-132 | 1-OCH₃, 3-Cl | CH₃ | H |
| A-133 | 1-F, 3-Br | CH₃ | H |
| A-134 | 1-Br, 3-F | CH₃ | H |
| A-135 | 1-F, 3-CF₃ | CH₃ | H |
| A-136 | 1-CF₃, 3-F | CH₃ | H |
| A-137 | 1-F, 3-OCH₃ | CH₃ | H |
| A-138 | 1-OCH₃, 3-F | CH₃ | H |
| A-139 | 1-Br, 3-CF3 | CH₃ | H |
| A-140 | 1-CF₃, 3-Br | CH₃ | H |
| A-141 | 1-Br, 3-OCH₃ | CH₃ | H |
| A-142 | 1-OCH₃, 3-Br | CH₃ | H |
| A-143 | 1-CF₃, 3-OCH₃ | CH₃ | H |
| A-144 | 1-OCH₃, 3-CF₃ | CH₃ | H |
| A-145 | 1-CH₃ | phenyl | H |
| A-146 | 2-CH₃ | phenyl | H |
| A-147 | 3-CH₃ | phenyl | H |
| A-148 | 4-CH₃ | phenyl | H |
| A-149 | 1-CH₃, 3-CH₃ | phenyl | H |
| A-150 | 1-CH₃, 3-CH₃, 4-CH₃ | phenyl | H |
| A-151 | 3-CH₃, 4-CH₃ | phenyl | H |
| A-152 | 1-Cl | phenyl | H |
| A-153 | 2-Cl | phenyl | H |
| A-154 | 3-Cl | phenyl | H |
| A-155 | 4-Cl | phenyl | H |
| A-156 | 1-Cl, 3-Cl | phenyl | H |
| A-157 | 1-Cl, 3-Cl, 4-Cl | phenyl | H |
| A-158 | 3-Cl, 4-Cl | phenyl | H |
| A-159 | 1-F | phenyl | H |
| A-160 | 2-F | phenyl | H |
| A-161 | 3-F | phenyl | H |
| A-162 | 4-F | phenyl | H |
| A-163 | 1-F, 3-F | phenyl | H |
| A-164 | 1-F, 3-F, 4-F | phenyl | H |
| A-165 | 3-F, 4-F | phenyl | H |
| A-166 | 1-Br | phenyl | H |
| A-167 | 2-Br | phenyl | H |
| A-168 | 3-Br | phenyl | H |
| A-169 | 4-Br | phenyl | H |
| A-170 | 1-Br, 3-Br | phenyl | H |
| A-171 | 1-Br, 3-Br, 4-Br | phenyl | H |
| A-172 | 3-Br, 4-Br | phenyl | H |
| A-173 | 1-CF₃ | phenyl | H |
| A-174 | 2-CF₃ | phenyl | H |
| A-175 | 3-CF₃ | phenyl | H |
| A-176 | 4-CF₃ | phenyl | H |
| A-177 | 1CF₃, 3-CF₃ | phenyl | H |
| A-178 | 1-CF₃, 3-CF₃, 4-CF₃ | phenyl | H |
| A-179 | 3-CF₃, 4-CF₃ | phenyl | H |
| A-180 | 1-OCH₃ | phenyl | H |
| A-181 | 2-OCH₃ | phenyl | H |
| A-182 | 3-OCH₃ | phenyl | H |
| A-183 | 4-OCH₃ | phenyl | H |
| A-184 | 1-OCH₃, 3-OCH₃ | phenyl | H |
| A-185 | 1-OCH₃, 3-OCH₃, 4-OCH₃ | phenyl | H |
| A-186 | 3,4-OCH₃, OCH₃ | phenyl | H |
| A-187 | 1-CH₃, 3-Cl | phenyl | H |
| A-188 | 1-Cl, 3-CH₃ | phenyl | H |
| A-189 | 1-CH₃, 3-F | phenyl | H |
| A-190 | 1-F, 3-CH₃ | phenyl | H |
| A-191 | 1-CH₃, 3-Br | phenyl | H |
| A-192 | 1-Br, 3-CH₃ | phenyl | H |
| A-193 | 1-CH₃, 3-CF₃ | phenyl | H |
| A-194 | 1-CF₃, 3-CH₃ | phenyl | H |
| A-195 | 1-CH₃, 3 OCH₃ | phenyl | H |
| A-196 | 1-OCH₃, 3-CH₃ | phenyl | H |
| A-197 | 1-Cl, 3-F | phenyl | H |
| A-198 | 1-F, 3-Cl | phenyl | H |
| A-199 | 1-Cl, 3-Br | phenyl | H |
| A-200 | 1-Br, 3-Cl | phenyl | H |
| A-201 | 1-Cl, 3-CF₃ | phenyl | H |
| A-202 | 11-CF₃, 3-Cl | phenyl | H |
| A-203 | 1-Cl, 3-OCH₃ | phenyl | H |
| A-204 | 1-OCH₃, 3-Cl | phenyl | H |
| A-205 | 1-F, 3-Br | phenyl | H |
| A-206 | 1-Br, 3-F | phenyl | H |
| A-207 | 1-F, 3-CF₃ | phenyl | H |
| A-208 | 1-CF₃, 3-F | phenyl | H |
| A-209 | 1-F, 3-OCH₃ | phenyl | H |
| A-210 | 1-OCH₃, 3-F | phenyl | H |
| A-211 | 1-Br, 3-CF₃ | phenyl | H |
| A-212 | 1-CF₃, 3-Br | phenyl | H |
| A-213 | 1-Br, 3-OCH₃ | phenyl | H |
| A-214 | 1-OCH₃, 3-Br | phenyl | H |
| A-215 | 1-CF₃, 3-OCH₃ | phenyl | H |
| A-216 | 1-OCH₃, 3-CF₃ | phenyl | H |
| A-217 | 1-CH₃ | benzyl | H |
| A-218 | 2-CH₃ | benzyl | H |
| A-219 | 3-CH₃ | benzyl | H |
| A-220 | 4-CH₃ | benzyl | H |
| A-221 | 1-CH₃, 3-CH₃ | benzyl | H |
| A-222 | 1-CH₃, 3-CH₃, 4-CH₃ | benzyl | H |
| A-223 | 3-CH₃, 4-CH₃ | benzyl | H |
| A-224 | 1-Cl | benzyl | H |
| A-225 | 2-Cl | benzyl | H |
| A-226 | 3-Cl | benzyl | H |
| A-227 | 4-Cl | benzyl | H |
| A-228 | 1-Cl, 3-Cl | benzyl | H |
| A-229 | 1-Cl, 3-Cl, 4-Cl | benzyl | H |
| A-230 | 3-Cl, 4-Cl | benzyl | H |
| A-231 | 1-F | benzyl | H |
| A-232 | 2-F | benzyl | H |
| A-233 | 3-F | benzyl | H |
| A-234 | 4-F | benzyl | H |
| A-235 | 1-F, 3-F | benzyl | H |
| A-236 | 1-F, 3-F, 4-F | benzyl | H |
| A-237 | 3-F, 4-F | benzyl | H |
| A-238 | 1-Br | benzyl | H |
| A-239 | 2-Br | benzyl | H |
| A-240 | 3-Br | benzyl | H |
| A-241 | 4-Br | benzyl | H |
| A-242 | 1-Br, 3-Br | benzyl | H |
| A-243 | 1-Br, 3-Br, 4-Br | benzyl | H |
| A-244 | 3-Br, 4-Br | benzyl | H |
| A-245 | 1-CF₃ | benzyl | H |
| A-246 | 2-CF₃ | benzyl | H |
| A-247 | 3-CF₃ | benzyl | H |
| A-248 | 4-CF₃ | benzyl | H |
| A-249 | 1CF₃, 3-CF₃ | benzyl | H |
| A-250 | 1-CF₃, 3-CF₃, 4-CF₃ | benzyl | H |
| A-251 | 3-CF₃, 4-CF₃ | benzyl | H |
| A-252 | 1-OCH₃ | benzyl | H |
| A-253 | 2-OCH₃ | benzyl | H |
| A-254 | 3-OCH₃ | benzyl | H |
| A-255 | 4-OCH₃ | benzyl | H |
| A-256 | 1-OCH₃, 3-OCH₃ | benzyl | H |
| A-257 | 1-OCH₃, 3-OCH₃, 4-OCH₃ | benzyl | H |
| A-258 | 3,4-OCH₃, OCH₃ | benzyl | H |
| A-259 | 1-CH₃, 3-Cl | benzyl | H |
| A-260 | 1-Cl, 3-CH₃ | benzyl | H |
| A-261 | 1-CH₃, 3-F | benzyl | H |
| A-262 | 1-F, 3-CH₃ | benzyl | H |
| A-263 | 1-CH₃, 3-Br | benzyl | H |
| A-264 | 1-Br, 3-CH₃ | benzyl | H |
| A-265 | 1-CH₃, 3-CF₃ | benzyl | H |
| A-266 | 1-CF₃, 3-CH₃ | benzyl | H |
| A-267 | 1-CH₃, 3 OCH₃ | benzyl | H |
| A-268 | 1-OCH₃, 3-CH₃ | benzyl | H |
| A-269 | 1-Cl, 3-F | benzyl | H |
| A-270 | 1-F, 3-Cl | benzyl | H |
| A-271 | 1-Cl, 3-Br | benzyl | H |
| A-272 | 1-Br, 3-Cl | benzyl | H |
| A-273 | 1-Cl, 3-CF₃ | benzyl | H |
| A-274 | 11-CF₃, 3-Cl | benzyl | H |
| A-275 | 1-Cl, 3-OCH₃ | benzyl | H |
| A-276 | 1-OCH₃, 3-Cl | benzyl | H |
| A-277 | 1-F, 3-Br | benzyl | H |
| A-278 | 1-Br, 3-F | benzyl | H |
| A-279 | 1-F, 3-CF₃ | benzyl | H |
| A-280 | 1-CF₃, 3-F | benzyl | H |
| A-281 | 1-F, 3-OCH₃ | benzyl | H |
| A-282 | 1-OCH₃, 3-F | benzyl | H |
| A-283 | 1-Br, 3-CF₃ | benzyl | H |
| A-284 | 1-CF₃, 3-Br | benzyl | H |
| A-285 | 1-Br, 3-OCH₃ | benzyl | H |
| A-286 | 1-OCH₃, 3-Br | benzyl | H |
| A-287 | 1-CF₃, 3-OCH₃ | benzyl | H |
| A-288 | 1-OCH₃, 3-CF₃ | benzyl | H |
| A-289 | 1-CH₃ | CHF₂ | H |
| A-290 | 2-CH₃ | CHF₂ | H |
| A-291 | 3-CH₃ | CHF₂ | H |
| A-292 | 4-CH₃ | CHF₂ | H |
| A-293 | 1-CH₃, 3-CH₃ | CHF₂ | H |
| A-294 | 1-CH₃, 3-CH₃, 4-CH₃ | CHF₂ | H |
| A-295 | 3-CH₃, 4-CH₃ | CHF₂ | H |
| A-296 | 1-Cl | CHF₂ | H |
| A-297 | 2-Cl | CHF₂ | H |
| A-298 | 3-Cl | CHF₂ | H |
| A-299 | 4-Cl | CHF₂ | H |
| A-300 | 1-Cl, 3-Cl | CHF₂ | H |
| A-301 | 1-Cl, 3-Cl, 4-Cl | CHF₂ | H |
| A-302 | 3-Cl, 4-Cl | CHF₂ | H |
| A-303 | 1-F | CHF₂ | H |
| A-304 | 2-F | CHF₂ | H |
| A-305 | 3-F | CHF₂ | H |
| A-306 | 4-F | CHF₂ | H |
| A-307 | 1-F, 3-F | CHF₂ | H |
| A-308 | 1-F, 3-F, 4-F | CHF₂ | H |
| A-309 | 3-F, 4-F | CHF₂ | H |
| A-310 | 1-Br | CHF₂ | H |
| A-311 | 2-Br | CHF₂ | H |
| A-312 | 3-Br | CHF₂ | H |
| A-313 | 4-Br | CHF₂ | H |
| A-314 | 1-Br, 3-Br | CHF₂ | H |
| A-315 | 1-Br, 3-Br, 4-Br | CHF₂ | H |
| A-316 | 3-Br, 4-Br | CHF₂ | H |
| A-317 | 1-CF₃ | CHF₂ | H |
| A-318 | 2-CF₃ | CHF₂ | H |
| A-319 | 3-CF₃ | CHF₂ | H |
| A-320 | 4-CF₃ | CHF₂ | H |
| A-321 | 1CF₃, 3-CF₃ | CHF₂ | H |
| A-322 | 1-CF₃, 3-CF₃, 4-CF₃ | CHF₂ | H |
| A-323 | 3-CF₃, 4-CF₃ | CHF₂ | H |
| A-324 | 1-OCH₃ | CHF₂ | H |
| A-325 | 2-OCH₃ | CHF₂ | H |
| A-326 | 3-OCH₃ | CHF₂ | H |
| A-327 | 4-OCH₃ | CHF₂ | H |
| A-328 | 1-OCH₃, 3-OCH₃ | CHF₂ | H |
| A-329 | 1-OCH₃, 3-OCH₃, 4-OCH₃ | CHF₂ | H |
| A-330 | 3,4-OCH₃, OCH₃ | CHF₂ | H |
| A-331 | 1-CH₃, 3-Cl | CHF₂ | H |
| A-332 | 1-Cl, 3-CH₃ | CHF₂ | H |
| A-333 | 1-CH₃, 3-F | CHF₂ | H |
| A-334 | 1-F, 3-CH₃ | CHF₂ | H |
| A-335 | 1-CH₃, 3-Br | CHF₂ | H |
| A-336 | 1-Br, 3-CH₃ | CHF₂ | H |
| A-337 | 1-CH₃, 3-CF₃ | CHF₂ | H |
| A-338 | 1-CF₃, 3-CH₃ | CHF₂ | H |
| A-339 | 1-CH₃, 3 OCH₃ | CHF₂ | H |
| A-340 | 1-OCH₃, 3-CH₃ | CHF₂ | H |
| A-341 | 1-Cl, 3-F | CHF₂ | H |
| A-342 | 1-F, 3-Cl | CHF₂ | H |
| A-343 | 1-Cl, 3-Br | CHF₂ | H |
| A-344 | 1-Br, 3-Cl | CHF₂ | H |
| A-345 | 1-Cl, 3-CF₃ | CHF₂ | H |
| A-346 | 11-CF₃, 3-Cl | CHF₂ | H |
| A-347 | 1-Cl, 3-OCH₃ | CHF₂ | H |
| A-348 | 1-OCH₃, 3-Cl | CHF₂ | H |
| A-349 | 1-F, 3-Br | CHF₂ | H |
| A-350 | 1-Br, 3-F | CHF₂ | H |
| A-351 | 1-F, 3-CF₃ | CHF₂ | H |
| A-352 | 1-CF₃, 3-F | CHF₂ | H |
| A-353 | 1-F, 3-OCH₃ | CHF₂ | H |
| A-354 | 1-OCH₃, 3-F | CHF₂ | H |
| A-355 | 1-Br, 3-CF₃ | CHF₂ | H |
| A-356 | 1-CF₃, 3-Br | CHF₂ | H |
| A-357 | 1-Br, 3-OCH₃ | CHF₂ | H |
| A-358 | 1-OCH₃, 3-Br | CHF₂ | H |
| A-359 | 1-CF₃, 3-OCH₃ | CHF₂ | H |
| A-360 | 1-OCH₃, 3-CF₃ | CHF₂ | H |
| A-361 | 1-CH₃ | CF₃ | H |
| A-362 | 2-CH₃ | CF₃ | H |
| A-363 | 3-CH₃ | CF₃ | H |
| A-364 | 4-CH₃ | CF₃ | H |
| A-365 | 1-CH₃, 3-CH₃ | CF₃ | H |
| A-366 | 1-CH₃, 3-CH₃, 4-CH₃ | CF₃ | H |
| A-367 | 3-CH₃, 4-CH₃ | CF₃ | H |
| A-368 | 1-Cl | CF₃ | H |
| A-369 | 2-Cl | CF₃ | H |
| A-370 | 3-Cl | CF₃ | H |
| A-371 | 4-Cl | CF₃ | H |
| A-372 | 1-Cl, 3-Cl | CF₃ | H |
| A-373 | 1-Cl, 3-Cl, 4-Cl | CF₃ | H |
| A-374 | 3-Cl, 4-Cl | CF₃ | H |
| A-375 | 1-F | CF₃ | H |
| A-376 | 2-F | CF₃ | H |
| A-377 | 3-F | CF₃ | H |
| A-378 | 4-F | CF₃ | H |
| A-379 | 1-F, 3-F | CF₃ | H |
| A-380 | 1-F, 3-F, 4-F | CF₃ | H |
| A-381 | 3-F, 4-F | CF₃ | H |
| A-382 | 1-Br | CF₃ | H |
| A-383 | 2-Br | CF₃ | H |
| A-384 | 3-Br | CF₃ | H |
| A-385 | 4-Br | CF₃ | H |
| A-386 | 1-Br, 3-Br | CF₃ | H |
| A-387 | 1-Br, 3-Br, 4-Br | CF₃ | H |
| A-388 | 3-Br, 4-Br | CF₃ | H |
| A-389 | 1-CF₃ | CF₃ | H |
| A-390 | 2-CF₃ | CF₃ | H |
| A-391 | 3-CF₃ | CF₃ | H |
| A-392 | 4-CF₃ | CF₃ | H |
| A-393 | 1CF₃, 3-CF₃ | CF₃ | H |
| A-394 | 1-CF₃, 3-CF₃, 4-CF₃ | CF₃ | H |
| A-395 | 3-CF₃, 4-CF3 | CF3 | H |
| A-396 | 1-OCH₃ | CF₃ | H |
| A-397 | 2-OCH₃ | CF₃ | H |
| A-398 | 3-OCH₃ | CF₃ | H |
| A-399 | 4-OCH₃ | CF₃ | H |
| A-400 | 1-OCH₃, 3-OCH₃ | CF₃ | H |
| A-401 | 1-OCH₃, 3-OCH₃, 4-OCH₃ | CF₃ | H |
| A-402 | 3,4-OCH₃, OCH₃ | CF₃ | H |
| A-403 | 1-CH₃, 3-Cl | CF₃ | H |
| A-404 | 1-Cl, 3-CH₃ | CF₃ | H |
| A-405 | 1-CH₃, 3-F | CF₃ | H |
| A-406 | 1-F, 3-CH₃ | CF₃ | H |
| A-407 | 1-CH₃, 3-Br | CF₃ | H |
| A-408 | 1-Br, 3-CH₃ | CF₃ | H |
| A-409 | 1-CH₃, 3-CF₃ | CF₃ | H |
| A-410 | 1-CF₃, 3-CH₃ | CF₃ | H |
| A-411 | 1-CH₃, 3 OCH₃ | CF₃ | H |
| A-412 | 1-OCH₃, 3-CH₃ | CF₃ | H |
| A-413 | 1-Cl, 3-F | CF₃ | H |
| A-414 | 1-F, 3-Cl | CF₃ | H |
| A-415 | 1-Cl, 3-Br | CF₃ | H |
| A-416 | 1-Br, 3-Cl | CF₃ | H |
| A-417 | 1-Cl, 3-CF₃ | CF₃ | H |
| A-418 | 11-CF₃, 3-Cl | CF₃ | H |
| A-419 | 1-Cl, 3-OCH₃ | CF₃ | H |
| A-420 | 1-OCH₃, 3-Cl | CF₃ | H |
| A-421 | 1-F, 3-Br | CF₃ | H |
| A-422 | 1-Br, 3-F | CF₃ | H |
| A-423 | 1-F, 3-CF₃ | CF₃ | H |
| A-424 | 1-CF₃, 3-F | CF₃ | H |
| A-425 | 1-F, 3-OCH₃ | CF₃ | H |
| A-426 | 1-OCH₃, 3-F | CF₃ | H |
| A-427 | 1-Br, 3-CF₃ | CF₃ | H |
| A-428 | 1-CF₃, 3-Br | CF₃ | H |
| A-429 | 1-Br, 3-OCH₃ | CF₃ | H |
| A-430 | 1-OCH₃, 3-Br | CF₃ | H |
| A-431 | 1-CF₃, 3-OCH₃ | CF₃ | H |
| A-432 | 1-OCH₃, 3-CF₃ | CF₃ | H |
| A-433 | 1-CH₃ | CH₃ | CH₃ |
| A-434 | 2-CH₃ | CH₃ | CH₃ |
| A-435 | 3-CH₃ | CH₃ | CH₃ |
| A-436 | 4-CH₃ | CH₃ | CH₃ |
| A-437 | 1-CH₃, 3-CH₃ | CH₃ | CH₃ |
| A-438 | 1-CH₃, 3-CH₃, 4-CH₃ | CH₃ | CH₃ |
| A-439 | 3-CH₃, 4-CH₃ | CH₃ | CH₃ |
| A-440 | 1-Cl | CH₃ | CH₃ |
| A-441 | 2-Cl | CH₃ | CH₃ |
| A-442 | 3-Cl | CH₃ | CH₃ |
| A-443 | 4-Cl | CH₃ | CH₃ |
| A-444 | 1-Cl, 3-Cl | CH₃ | CH₃ |
| A-445 | 1-Cl, 3-Cl, 4-Cl | CH₃ | CH₃ |
| A-446 | 3-Cl, 4-Cl | CH₃ | CH₃ |
| A-447 | 1-F | CH₃ | CH₃ |
| A-448 | 2-F | CH₃ | CH₃ |
| A-449 | 3-F | CH₃ | CH₃ |
| A-450 | 4-F | CH₃ | CH₃ |
| A-451 | 1-F, 3-F | CH₃ | CH₃ |
| A-452 | 1-F, 3-F, 4-F | CH₃ | CH₃ |
| A-453 | 3-F, 4-F | CH₃ | CH₃ |
| A-454 | 1-Br | CH₃ | CH₃ |
| A-455 | 2-Br | CH₃ | CH₃ |
| A-456 | 3-Br | CH₃ | CH₃ |
| A-457 | 4-Br | CH₃ | CH₃ |
| A-458 | 1-Br, 3-Br | CH₃ | CH₃ |
| A-459 | 1-Br, 3-Br, 4-Br | CH₃ | CH₃ |
| A-460 | 3-Br, 4-Br | CH₃ | CH₃ |
| A-461 | 1-CF₃ | CH₃ | CH₃ |
| A-462 | 2-CF₃ | CH₃ | CH₃ |
| A-463 | 3-CF₃ | CH₃ | CH₃ |
| A-464 | 4-CF₃ | CH₃ | CH₃ |
| A-465 | 1CF₃, 3-CF₃ | CH₃ | CH₃ |
| A-466 | 1-CF₃, 3-CF₃, 4-CF₃ | CH₃ | CH₃ |
| A-467 | 3-CF₃, 4-CF3 | CH₃ | CH₃ |
| A-468 | 1-OCH₃ | CH₃ | CH₃ |
| A-469 | 2-OCH₃ | CH₃ | CH₃ |
| A-470 | 3-OCH₃ | CH₃ | CH₃ |
| A-471 | 4-OCH₃ | CH₃ | CH₃ |
| A-472 | 1-OCH₃, 3-OCH₃ | CH₃ | CH₃ |
| A-473 | 1-OCH₃, 3-OCH₃, 4-OCH₃ | CH₃ | CH₃ |
| A-474 | 3,4-OCH₃, OCH₃ | CH₃ | CH₃ |
| A-475 | 1-CH₃, 3-Cl | CH₃ | CH₃ |
| A-476 | 1-Cl, 3-CH₃ | CH₃ | CH₃ |
| A-477 | 1-CH₃, 3-F | CH₃ | CH₃ |
| A-478 | 1-F, 3-CH₃ | CH₃ | CH₃ |
| A-479 | 1-CH₃, 3-Br | CH₃ | CH₃ |
| A-480 | 1-Br, 3-CH₃ | CH₃ | CH₃ |
| A-481 | 1-CH₃, 3-CF₃ | CH₃ | CH₃ |
| A-482 | 1-CF₃, 3-CH₃ | CH₃ | CH₃ |
| A-483 | 1-CH₃, 3 OCH₃ | CH₃ | CH₃ |
| A-484 | 1-OCH₃, 3-CH₃ | CH₃ | CH₃ |
| A-485 | 1-Cl, 3-F | CH₃ | CH₃ |
| A-486 | 1-F, 3-Cl | CH₃ | CH₃ |
| A-487 | 1-Cl, 3-Br | CH₃ | CH₃ |
| A-488 | 1-Br, 3-Cl | CH₃ | CH₃ |
| A-489 | 1-Cl, 3-CF3 | CH₃ | CH₃ |
| A-490 | 11-CF₃, 3-Cl | CH₃ | CH₃ |
| A-491 | 1-Cl, 3-OCH₃ | CH₃ | CH₃ |
| A-492 | 1-OCH₃, 3-Cl | CH₃ | CH₃ |
| A-493 | 1-F, 3-Br | CH₃ | CH₃ |
| A-494 | 1-Br, 3-F | CH₃ | CH₃ |
| A-495 | 1-F, 3-CF₃ | CH₃ | CH₃ |
| A-496 | 1-CF₃, 3-F | CH₃ | CH₃ |
| A-497 | 1-F, 3-OCH₃ | CH₃ | CH₃ |
| A-498 | 1-OCH₃, 3-F | CH₃ | CH₃ |
| A-499 | 1-Br, 3-CF3 | CH₃ | CH₃ |
| A-500 | 1-CF₃, 3-Br | CH₃ | CH₃ |
| A-501 | 1-Br, 3-OCH₃ | CH₃ | CH₃ |
| A-502 | 1-OCH₃, 3-Br | CH₃ | CH₃ |
| A-503 | 1-CF₃, 3-OCH₃ | CH₃ | CH₃ |
| A-504 | 1-OCH₃, 3-CF₃ | CH₃ | CH₃ |
| A-505 | 1-CH₃ | phenyl | CH₃ |
| A-506 | 2-CH₃ | phenyl | CH₃ |
| A-507 | 3-CH₃ | phenyl | CH₃ |
| A-508 | 4-CH₃ | phenyl | CH₃ |
| A-509 | 1-CH₃, 3-CH₃ | phenyl | CH₃ |
| A-510 | 1-CH₃, 3-CH₃, 4-CH₃ | phenyl | CH₃ |
| A-511 | 3-CH₃, 4-CH₃ | phenyl | CH₃ |
| A-512 | 1-Cl | phenyl | CH₃ |
| A-513 | 2-Cl | phenyl | CH₃ |
| A-514 | 3-Cl | phenyl | CH₃ |
| A-515 | 4-Cl | phenyl | CH₃ |
| A-516 | 1-Cl, 3-Cl | phenyl | CH₃ |
| A-517 | 1-Cl, 3-Cl, 4-Cl | phenyl | CH₃ |
| A-518 | 3-Cl, 4-Cl | phenyl | CH₃ |
| A-519 | 1-F | phenyl | CH₃ |
| A-520 | 2-F | phenyl | CH₃ |
| A-521 | 3-F | phenyl | CH₃ |
| A-522 | 4-F | phenyl | CH₃ |
| A-523 | 1-F, 3-F | phenyl | CH₃ |
| A-524 | 1-F, 3-F, 4-F | phenyl | CH₃ |
| A-525 | 3-F, 4-F | phenyl | CH₃ |
| A-526 | 1-Br | phenyl | CH₃ |
| A-527 | 2-Br | phenyl | CH₃ |
| A-528 | 3-Br | phenyl | CH₃ |
| A-529 | 4-Br | phenyl | CH₃ |
| A-530 | 1-Br, 3-Br | phenyl | CH₃ |
| A-531 | 1-Br, 3-Br, 4-Br | phenyl | CH₃ |
| A-532 | 3-Br, 4-Br | phenyl | CH₃ |
| A-533 | 1-CF₃ | phenyl | CH₃ |
| A-534 | 2-CF₃ | phenyl | CH₃ |
| A-535 | 3-CF₃ | phenyl | CH₃ |
| A-536 | 4-CF₃ | phenyl | CH₃ |
| A-537 | 1CF₃, 3-CF₃ | phenyl | CH₃ |
| A-538 | 1-CF₃, 3-CF₃, 4-CF₃ | phenyl | CH₃ |
| A-539 | 3-CF₃, 4-CF3 | phenyl | CH₃ |
| A-540 | 1-OCH₃ | phenyl | CH₃ |
| A-541 | 2-OCH₃ | phenyl | CH₃ |
| A-542 | 3-OCH₃ | phenyl | CH₃ |
| A-543 | 4-OCH₃ | phenyl | CH₃ |
| A-544 | 1-OCH₃, 3-OCH₃ | phenyl | CH₃ |
| A-545 | 1-OCH₃, 3-OCH₃, 4-OCH₃ | phenyl | CH₃ |
| A-546 | 3,4-OCH₃, OCH₃ | phenyl | CH₃ |
| A-547 | 1-CH₃, 3-Cl | phenyl | CH₃ |
| A-548 | 1-Cl, 3-CH₃ | phenyl | CH₃ |
| A-549 | 1-CH₃, 3-F | phenyl | CH₃ |
| A-550 | 1-F, 3-CH₃ | phenyl | CH₃ |
| A-551 | 1-CH₃, 3-Br | phenyl | CH₃ |
| A-552 | 1-Br, 3-CH₃ | phenyl | CH₃ |
| A-553 | 1-CH₃, 3-CF₃ | phenyl | CH₃ |
| A-554 | 1-CF₃, 3-CH₃ | phenyl | CH₃ |
| A-555 | 1-CH₃, 3 OCH₃ | phenyl | CH₃ |
| A-556 | 1-OCH₃, 3-CH₃ | phenyl | CH₃ |
| A-557 | 1-Cl, 3-F | phenyl | CH₃ |
| A-558 | 1-F, 3-Cl | phenyl | CH₃ |
| A-559 | 1-Cl, 3-Br | phenyl | CH₃ |
| A-560 | 1-Br, 3-Cl | phenyl | CH₃ |
| A-561 | 1-Cl, 3-CF3 | phenyl | CH₃ |
| A-562 | 11-CF₃, 3-Cl | phenyl | CH₃ |
| A-563 | 1-Cl, 3-OCH₃ | phenyl | CH₃ |
| A-564 | 1-OCH₃, 3-Cl | phenyl | CH₃ |
| A-565 | 1-F, 3-Br | phenyl | CH₃ |
| A-566 | 1-Br, 3-F | phenyl | CH₃ |
| A-567 | 1-F, 3-CF₃ | phenyl | CH₃ |
| A-568 | 1-CF₃, 3-F | phenyl | CH₃ |
| A-569 | 1-F, 3-OCH₃ | phenyl | CH₃ |
| A-570 | 1-OCH₃, 3-F | phenyl | CH₃ |
| A-571 | 1-Br, 3-CF3 | phenyl | CH₃ |
| A-572 | 1-CF₃, 3-Br | phenyl | CH₃ |
| A-573 | 1-Br, 3-OCH₃ | phenyl | CH₃ |
| A-574 | 1-OCH₃, 3-Br | phenyl | CH₃ |
| A-575 | 1-CF₃, 3-OCH₃ | phenyl | CH₃ |
| A-576 | 1-OCH₃, 3-CF₃ | phenyl | CH₃ |
| A-577 | 1-CH₃ | benzyl | CH₃ |
| A-578 | 2-CH₃ | benzyl | CH₃ |
| A-579 | 3-CH₃ | benzyl | CH₃ |
| A-580 | 4-CH₃ | benzyl | CH₃ |
| A-581 | 1-CH₃, 3-CH₃ | benzyl | CH₃ |
| A-582 | 1-CH₃, 3-CH₃, 4-CH₃ | benzyl | CH₃ |
| A-583 | 3-CH₃, 4-CH₃ | benzyl | CH₃ |
| A-584 | 1-Cl | benzyl | CH₃ |
| A-585 | 2-Cl | benzyl | CH₃ |
| A-586 | 3-Cl | benzyl | CH₃ |
| A-587 | 4-Cl | benzyl | CH₃ |
| A-588 | 1-Cl, 3-Cl | benzyl | CH₃ |
| A-589 | 1-Cl, 3-Cl, 4-Cl | benzyl | CH₃ |
| A-590 | 3-Cl, 4-Cl | benzyl | CH₃ |
| A-591 | 1-F | benzyl | CH₃ |
| A-592 | 2-F | benzyl | CH₃ |
| A-593 | 3-F | benzyl | CH₃ |
| A-594 | 4-F | benzyl | CH₃ |
| A-595 | 1-F, 3-F | benzyl | CH₃ |
| A-596 | 1-F, 3-F, 4-F | benzyl | CH₃ |
| A-597 | 3-F, 4-F | benzyl | CH₃ |
| A-598 | 1-Br | benzyl | CH₃ |
| A-599 | 2-Br | benzyl | CH₃ |
| A-600 | 3-Br | benzyl | CH₃ |
| A-601 | 4-Br | benzyl | CH₃ |
| A-602 | 1-Br, 3-Br | benzyl | CH₃ |
| A-603 | 1-Br, 3-Br, 4-Br | benzyl | CH₃ |
| A-604 | 3-Br, 4-Br | benzyl | CH₃ |
| A-605 | 1-CF₃ | benzyl | CH₃ |
| A-606 | 2-CF₃ | benzyl | CH₃ |
| A-607 | 3-CF₃ | benzyl | CH₃ |
| A-608 | 4-CF₃ | benzyl | CH₃ |
| A-609 | 1CF₃, 3-CF₃ | benzyl | CH₃ |
| A-610 | 1-CF₃, 3-CF₃, 4-CF₃ | benzyl | CH₃ |
| A-611 | 3-CF₃, 4-CF3 | benzyl | CH₃ |
| A-612 | 1-OCH₃ | benzyl | CH₃ |
| A-613 | 2-OCH₃ | benzyl | CH₃ |
| A-614 | 3-OCH₃ | benzyl | CH₃ |
| A-615 | 4-OCH₃ | benzyl | CH₃ |
| A-616 | 1-OCH₃, 3-OCH₃ | benzyl | CH₃ |
| A-617 | 1-OCH₃, 3-OCH₃, 4-OCH₃ | benzyl | CH₃ |
| A-618 | 3,4-OCH₃, OCH₃ | benzyl | CH₃ |
| A-619 | 1-CH₃, 3-Cl | benzyl | CH₃ |
| A-620 | 1-Cl, 3-CH₃ | benzyl | CH₃ |
| A-621 | 1-CH₃, 3-F | benzyl | CH₃ |
| A-622 | 1-F, 3-CH₃ | benzyl | CH₃ |
| A-623 | 1-CH₃, 3-Br | benzyl | CH₃ |
| A-624 | 1-Br, 3-CH₃ | benzyl | CH₃ |
| A-625 | 1-CH₃, 3-CF₃ | benzyl | CH₃ |
| A-626 | 1-CF₃, 3-CH₃ | benzyl | CH₃ |
| A-627 | 1-CH₃, 3 OCH₃ | benzyl | CH₃ |
| A-628 | 1-OCH₃, 3-CH₃ | benzyl | CH₃ |
| A-629 | 1-Cl, 3-F | benzyl | CH₃ |
| A-630 | 1-F, 3-Cl | benzyl | CH₃ |
| A-631 | 1-Cl, 3-Br | benzyl | CH₃ |
| A-632 | 1-Br, 3-Cl | benzyl | CH₃ |
| A-633 | 1-Cl, 3-CF3 | benzyl | CH₃ |
| A-634 | 11-CF₃, 3-Cl | benzyl | CH₃ |
| A-635 | 1-Cl, 3-OCH₃ | benzyl | CH₃ |
| A-636 | 1-OCH₃, 3-Cl | benzyl | CH₃ |
| A-637 | 1-F, 3-Br | benzyl | CH₃ |
| A-638 | 1-Br, 3-F | benzyl | CH₃ |
| A-639 | 1-F, 3-CF₃ | benzyl | CH₃ |
| A-640 | 1-CF₃, 3-F | benzyl | CH₃ |
| A-641 | 1-F, 3-OCH₃ | benzyl | CH₃ |
| A-642 | 1-OCH₃, 3-F | benzyl | CH₃ |
| A-643 | 1-Br, 3-CF3 | benzyl | CH₃ |
| A-644 | 1-CF₃, 3-Br | benzyl | CH₃ |
| A-645 | 1-Br, 3-OCH₃ | benzyl | CH₃ |
| A-646 | 1-OCH₃, 3-Br | benzyl | CH₃ |
| A-647 | 1-CF₃, 3-OCH₃ | benzyl | CH₃ |
| A-648 | 1-OCH₃, 3-CF₃ | benzyl | CH₃ |

TABLE B

| | R^{T3} |
|---|---|
| B-1 | methyl |
| B-2 | ethyl |
| B-3 | n-propyl |
| B-4 | isopropyl |
| B-5 | n-butyl |
| B-6 | sec-btyl |
| B-7 | isobutyl |
| B-8 | tert-butyl |
| B-9 | benzyl |
| B-10 | diphenylmethyl |
| B-11 | 1-phenylethyl |
| B-12 | 2-phenylethyl |
| B-13 | 1-phenylpropyl |
| B-14 | 2-phenylpropyl |
| B-15 | 3-phenylpropyl |
| B-16 | furan-2-ylmethyl |
| B-17 | thien-2-ylmethyl |
| B-18 | pyrrol-2-ylmethyl |
| B-19 | pyrazol-3-ylmethyl |
| B-20 | imidazol-2-ylmethyl |
| B-21 | oxazol-2-ylmethyl |
| B-22 | thiazol-2-ylmethyl |
| B-23 | pyrid-2-ylmethyl |
| B-24 | pyrid-3-ylmethyl |
| B-25 | pyrid-4-ylmethyl |
| B-26 | pyrimidin-2-ylmethyl |
| B-27 | pyrimidin-4-ylmethyl |
| B-28 | methoxymethyl |
| B-29 | 1-methoxyethyl |
| B-30 | 2-methoxyethyl |
| B-31 | phenoxymethyl |
| B-32 | 1-phenoxyethyl |
| B-33 | 2-phenoxyethyl |
| B-34 | methylthiomethyl |
| B-35 | 1-methylthioethyl |
| B-36 | 2-methylthioethyl |
| B-37 | acetylmethyl |
| B-38 | acetyloxymethyl |
| B-39 | 1-acetylethyl |
| B-40 | 2-acetylethyl |
| B-41 | 1-acetyloxyethyl |
| B-42 | 2-acetyloxyethyl |
| B-43 | 2-acetylprop-1-yl |
| B-44 | 2-acetylprop-2-yl |
| B-45 | 2-acetyloxyprop-1-yl |
| B-46 | 2-acetyloxyprop-2-yl |
| B-47 | cyclopentyl |
| B-48 | cyclohexyl |
| B-49 | phenyl |
| B-50 | 2-methylphenyl |
| B-51 | 3-methylphenyl |
| B-52 | 4-methylphenyl |
| B-53 | 2-(trifluoromethyl)-phenyl |
| B-54 | 3-(trifluoromethyl)-phenyl |
| B-55 | 4-(trifluoromethyl)-phenyl |
| B-56 | 2-chlorophenyl |
| B-57 | 3-chlorophenyl |
| B-58 | 4-chlorophenyl |
| B-59 | 2-fluorophenyl |
| B-60 | 3-fluorophenyl |
| B-61 | 4-fluorophenyl |
| B-62 | 2-acetylphenyl |
| B-63 | 3-acetylphenyl |
| B-64 | 4-acetylphenyl |
| B-65 | 2-acetyloxyphenyl |
| B-66 | 3-acetyloxyphenyl |
| B-67 | 4-acetyloxyphenyl |
| B-68 | pyrid-2-yl |
| B-69 | pyrid-3-yl |
| B-70 | pyrid-4-yl |
| B-71 | 2-fluoropyrid-3-yl |
| B-72 | 4-fluoropyrid-3-yl |
| B-73 | 5-fluoropyrid-3-yl |
| B-74 | 6-fluoropyrid-3-yl |
| B-75 | 2-chloropyrid-3-yl |
| B-76 | 4-chloropyrid-3-yl |
| B-77 | 5-chloropyrid-3-yl |
| B-78 | 6-chloropyrid-3-yl |
| B-79 | 2-bromopyrid-3-yl |
| B-80 | 4-bromopyrid-3-yl |
| B-81 | 5-bromopyrid-3-yl |
| B-82 | 6-bromopyrid-3-yl |
| B-83 | 2-methylsulfanylpyrid-3-yl |
| B-84 | 4-methylsulfanylpyrid-3-yl |
| B-85 | 5-methylsulfanylpyrid-3-yl |
| B-86 | 6-methylsulfanylpyrid-3-yl |
| B-87 | 2-ethylsulfanylpyrid-3-yl |
| B-88 | 4-ethylsulfanylpyrid-3-yl |
| B-89 | 5-ethylsulfanylpyrid-3-yl |
| B-90 | 6-ethylsulfanylpyrid-3-yl |
| B-91 | 2-naphthyl |
| B-92 | benzo-1,3-dioxolan-5-yl |
| B-93 | furan-2-yl |
| B-94 | furan-3-yl |
| B-95 | 2-methylfuran-3-yl |
| B-96 | 5-methylfuran-3-yl |
| B-97 | 2,5-dimethylfuran-3-yl |
| B-98 | 5-methylfuran-2-yl |
| B-99 | 5-chlorofuran-2-yl |
| B-100 | thien-2-yl |
| B-101 | thien-3-yl |
| B-102 | 2-methylthien-3-yl |
| B-103 | 5-methylthien-3-yl |
| B-104 | 2,5-dimethylthien-3-yl |
| B-105 | 5-methylthien-2-yl |
| B-106 | 5-chlorothien-2-yl |
| B-107 | oxazol-2-yl |
| B-108 | oxazol-4-yl |
| B-109 | oxazol-5-yl |
| B-110 | isoxazol-3-yl |
| B-111 | isoxazol-4-yl |
| B-112 | isoxazol-5-yl |
| B-113 | 5-methyl-3-(2-chlorophenyl)- |
| | isoxazol-4-yl |
| B-114 | pyrazol-3-yl |
| B-115 | pyrazol-4-yl |
| B-116 | pyrazol-5-yl |
| B-117 | 1-methylpyrazol-3-yl |
| B-118 | 1-methyl-5- |
| | ethylcarbonyloxypyrazol-4-yl |

The compounds of formula (I) can be obtained as outlined in schemes 1 to 14.

In the following, compounds (I) wherein Z is a group (II.A) are named compounds (I.A), compounds (I) wherein Z is a group (II.B) are named compounds (I.B) and compounds (I) wherein Z is a group (11.0) are named compounds (1.0).

The compounds of the formula (I.A) according to the invention wherein X is oxygen or sulphur and R^{2a} is hydrogen (referred herein as compounds I.A.a) can be prepared e.g. from the corresponding urea compounds IV and thiourea compounds III, respectively, as shown in scheme 1.

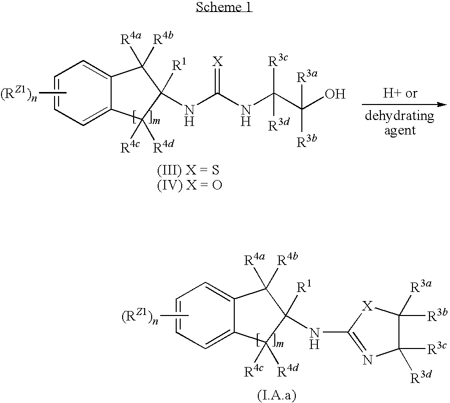

The variables R¹, R^{3a}, R^{3b}, R^{3c}, R^{3d}, R^{4a}, R^{4b}, R^{4c}, R^{4d}, R^{Z1}, m and n have the meanings given above.

The thiourea compound (III) and the urea compound (IV), respectively, can be cyclized by conventional means thereby obtaining the azoline compound of the formula (I.A.a). Cyclization of the compound (III) and (IV), respectively, can be achieved e.g. under acid catalysis or under dehydrating conditions e.g. by Mitsunobu's reaction (see Tetrahedron Letters 1999, 40, 3125-3128).

Alternatively, the cyclization can be carried out in the presence of a trialkyl- or tricycloalkylphosphine, such as trimethylphosphine, triethylphosphine, tripropylphosphine, triisopropylphosphine, tributylphosphine or tricyclohexylphosphine, a triarylphosphine, such as triphenylphosphine, or, preferably, in the presence of a phosphonium salt of the formula [P(R^{α})₃R^{β}]⁺ A⁻ wherein R^{α} is C₁-C₄-alkyl, cyclohexyl or phenyl; R^{β} is CH₂-U, where U is C₁-C₄-alylcarbonyl, C₁-C₄-alkoxycarbonyl or, preferably, CN; and A is an anion equivalent, for example a halide anion. A particularly suited phosphorus reagent is cyanomethyltrimethylphosphonium iodide (see Tetrahedron 2001, 57, 5451-54). Moreover, this variant is suitably carried out in the presence of a base, which is suitably an amine, such as triethylamine or diisopropylethylamine. This variant can be carried out in analogy to the method described in US 2005/0032814.

Alternatively, the compounds of the formula (I.A) according to the invention wherein X is O or S and R^{2a}, R^{3a}, R^{3b}, R^{3c} and R^{3d} are hydrogen (referred herein as compounds (I.A.b)) can be prepared by the method shown in scheme 2.

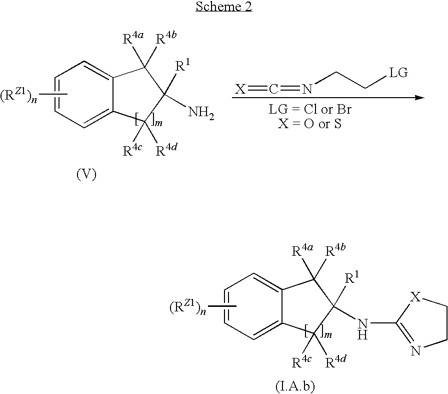

The variables R¹, R^{4a}, R^{4b}, R^{4c}, R^{4d}, R^{Z1}, m and n have the meanings given above.

An amine V or a salt thereof can be converted into an azoline (I.A.b) by reaction with 2-chloroethylisothiocyanate or 2-chloroethylisocyanate (e.g. as described in Bioorg. Med. Chem. Lett. 1994, 4, 2317-22) and subsequent cyclization in the presence or absence of a base.

1-Chloro-2-isothiocyanatoethane (CAS 6099-88-3), 2-chloroethylisocyanate (CAS 1943-83-5), 1-bromo-2-isothiocyanatoethane (CAS 1483-41-6) and 2-bromoethylisocyanate (CAS 42865-19-0) are commercially available.

This method often affords as by-product a compound (I.B.1):

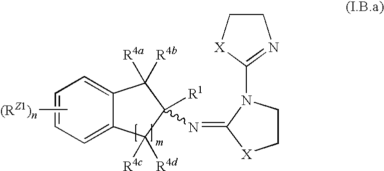

The variables R¹, R^{4a}, R^{4b}, R^{4c}, R^{4d}, R^{Z1}, m and n have the meanings given above. X is O or S.

Compounds of the formula (I.A) according to the invention wherein X is NR⁵ (referred herein as compounds (I.A.c)) may be prepared by the method outlined in scheme 3.

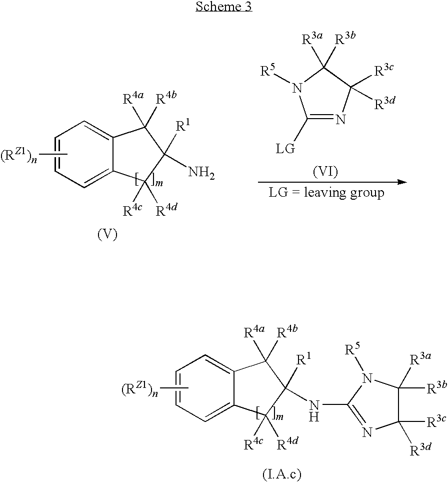

The variables R¹, R^{3a}, R^{3b}, R^{3c}, R^{3d}, R^{4a}, R^{4b}, R^{4c}, R^{4d}, R⁵, R^{Z1}, m and n have the meanings given above. LG is a leaving group, e.g. a halogen atom, a tosylate or a triflate group.

Compounds of the formula (I.A.c) may be obtained by reacting an appropriate substituted amine (V) or a salt thereof with a 2-substituted imidazoline (VI) in an appropriate solvent. This reaction can be carried out, for example analogous to the methods described in U.S. Pat. No. 5,130,441 or EP 0389765.

Amines (V) are known in the art or can be prepared by methods familiar to an organic chemist, for instance by application of general methods for the synthesis of amines. Suitable amine salts (V) are e.g. the acid addition salts formed by treating an amine (V) with an inorganic or organic acid. Anions of useful acids are e.g. sulfate, hydrogen sulfate, phosphate, dihydrogen phosphate, hydrogen phosphate, nitrate, bicarbonate, carbonate, chloride, bromide, p-toluene sulfonate, and the anions of C₁-C₄-alkanoic acids such as acetate, propionate, and the like.

For instance, amines (V) can be prepared from suitable ketones (VII) by the methods depicted in schemes 4 and 5 below.

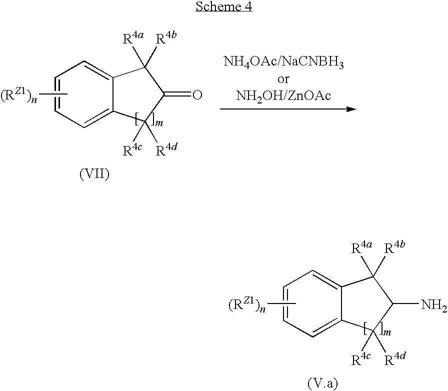

The variables R^{4a}, R^{4b}, R^{4c}, R^{4d}, R^{Z1}, m and n have the meanings given above.

Amines (V) wherein R¹ is hydrogen (V.a) can be obtained through reductive amination using e.g. NH₄OAc and NaCNBH₃ or NH₂OH/ZnOAc (see R. C. Larock, Comprehensive Organic Transformations 2^{nd} Ed., Wiley-VCH, 1999, p. 843-846). Alternatively, compounds (V.a) can be prepared by reduction of the ketone and subsequent amination of the resulting alcohol (see Mitsunobu, Hughes, Organic Reactions 1992, 42, 335-656).

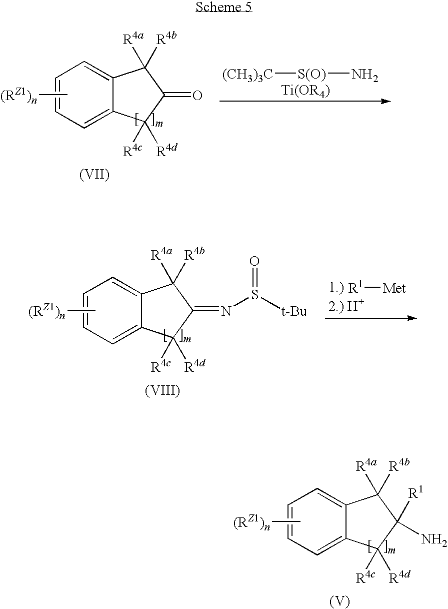

The variables R¹, R^{4a}, R^{4b}, R^{4c}, R^{4d}, R^{Z1}, m and n have the meanings given above. Met is a metalorganic residue, such as Mg-Hal (Hal=halogen), Li, Zn—R(R=alkyl) or SnR³ (R=alkyl), t-Bu is tert-butyl and R is C₁-C₆-alkyl.

Amines (V) wherein R¹ is different from hydrogen (V.b) can be obtained from the ketone (VII) via a two step synthesis. In a first step the ketone is reacted with a suitable sulfinamide such as (2-methyl-2-propane)sulfinamide in presence of a Lewis acid such as titanium tetraalkylate, e.g. titanium tetraisopropylate or titanium tetraethyl. The radical R¹ is subsequently introduced via a nucleophilic addition of a metallorganic compound R¹-Met followed by protonation and deprotection of the amino group.

Suitable ketones (VII) are known in the art or can be prepared by methods familiar to an organic chemist, for instance by application of general methods for the synthesis of ketones. Indanone (ketone (VII) wherein m is 1, n is 0 and R^{4a}, R^{4a}, R^{4a}, and R^{4a} are H) and β-tetralon (ketone (VII) wherein m is 2, n is 0 and R^{4a}, R^{4a}, R^{4a}, and R^{4a} are H) are commercially available. Schemes 6 and 7 below illustrate some synthetic routes towards substituted ketones (VII).

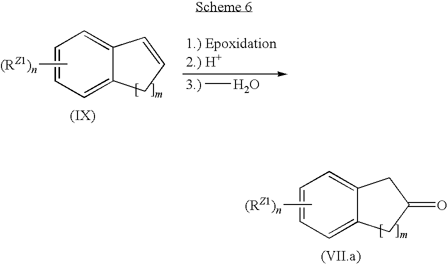

The variables R^{Z1}, m and n have the meanings given above.

Ketones (VII.a) wherein R^{4a}, R^{4b}, R^{4c} and R^{4d} are hydrogen can be prepared by epoxidation of an indene ((IX); m=1) or a dihydronaphthalene ((IX); m=2), acidic cleavage of the epoxide followed by elimination of water and tautomerization of the enol. Epoxidation and subsequent cleavage and water elimination can be carried out by standard procedures as described, for example, in J. March, Advanced Organic Chemistry, Wiley. Suitable epoxidation reagents are for example peracids, such as perfumaric acid, peracetic acid, perbenzoic acid and m-chloroperbenzoic acid.

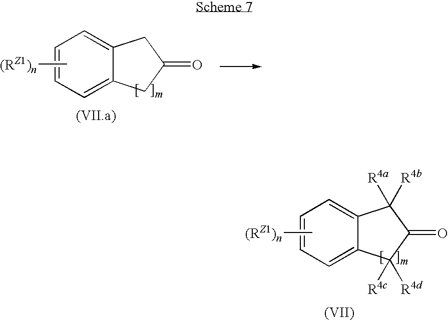

Ketones (VII) wherein at least one of the radicals R^{4a}, R^{4b}, R^{4c} and R^{4d} is different from hydrogen (if R^{4c} and/or R^{4d} are different from hydrogen, they are directly neighboured to the carbonyl group in case m is 2) can be obtained from a suitable α-unsubstituted ketone (VII.a) via alkylation or palladium-catalyzed arylation (see M. Palucki, S. L. Buchwald J. Am. Chem. Soc. 1997, 119, 11108-11109 or J. M. Fox, X. Huang, A. Chieffi, S. L. Buchwald J. Am. Chem. Soc. 2000, 122, 1360-1370) of the enolate species.

Alternatively, α-substituted ketones (VII) can be obtained from an intramolecular Friedel-Crafts-Acylation of a suitable aromatic acid chloride (see Y. Oshiro et al. J. Med. Chem. 1991, 34, 2004-2013 or W. Vaccaro et al. J. Med. Chem. 1996, 39, 1704-1719).

Compounds of the formula (I.A) and (I.B) wherein R^{2a} and R^{2b} are different from hydrogen can be obtained as outlined in scheme 8.

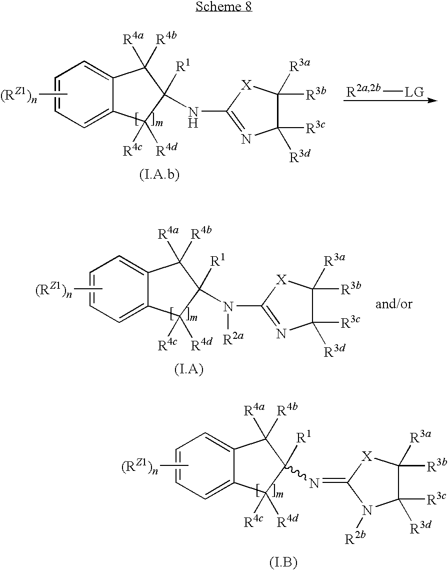

The variables X, R^{Z1}, R¹, R^{3a}, R^{3b}, R^{3c}, R^{3d}, R^{4a}, R^{4b}, R^{4c}, R^{4d}, m and n are as defined above. LG is a leaving group, e.g. a halide ion, such as CI or Br, a tosylate or a triflate group.

A compound (I.A.b) wherein R^{2a} and R^{2b}, respectively, are hydrogen is treated with a suitable electrophile. Suitable electrophiles are e.g. alkylating or acylating agents R^{2a,2b}-LG (LG=leaving group) e.g. as described in WO 2005/063724.

Compounds of the formula (III) and (IV), respectively, can be prepared as shown in schemes 9 and 10 below.

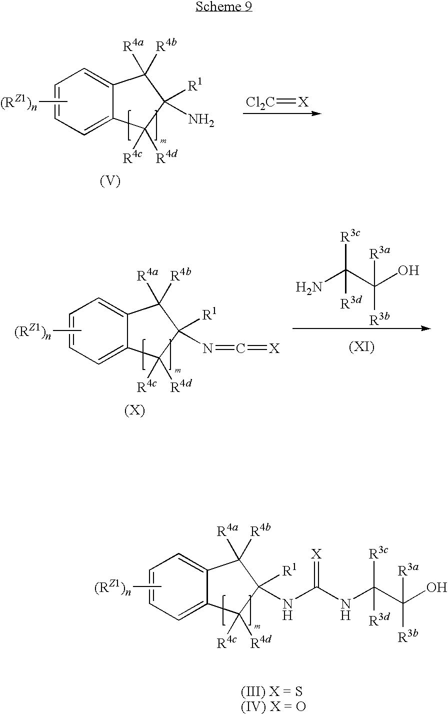

The variables R^{Z1}, R¹, R^{3a}, R^{3b}, R^{3c}, R^{3d}, R^{4a}, R^{4b}, R^{4c}, R^{4d}, m and n are as defined above. X is O or S.

An amine (V) or a salt thereof is converted to the corresponding iso(thio)cyanate (X) by conventional means, e.g. by reacting (V) with (thio)phosgene, as described for example in the case of thiophosgene in Houben-Weyl, E4, “Methoden der Organischen Chemie”, chapter IIc, pp. 837-842, Georg Thieme Verlag 1983. It may be advantageous to carry out the reaction in the presence of a base. The iso(thio)cyanate (X) is then reacted with an aminoethanol (XI) to form an amino(thio)carbonylaminoethane compound. The reaction of the aminoethanol (XI) with iso(thio)cyanate (X) can be performed in accordance with standard methods of organic chemistry, see e.g. Biosci. Biotech. Biochem. 56 (7), 1062-65 (1992).

Another method for preparing compounds of formula (III) wherein X is S is shown in scheme 10.

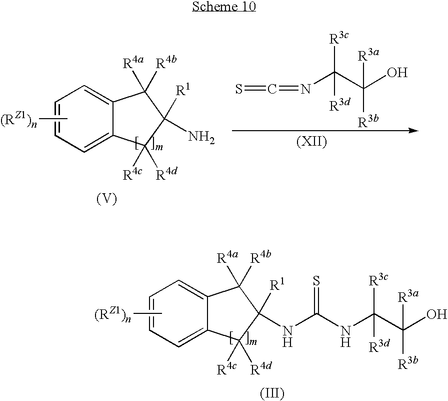

The variables R^{Z1}, R¹, R^{3a}, R^{3b}, R^{3c}, R^{3d}, R^{4a}, R^{4b}, R^{4c}, R^{4d}, m and n are as defined above.

An amine (V) or a salt thereof can be converted to the corresponding thiourea compound (III) by reaction of the amine (V) with an isothiocyanate (XII) and subsequent saponification (see G. Liu et al. J. Org. Chem. 1999, 64, 1278-1284). Isothiocyanates of formula (XII) can be prepared according to the procedures described in Coll. Czech. Chem. Commun. 1986, 51, 112-117.

Compounds III and IV belong to the group of compounds (I.C). They can be converted into compounds (I.C) wherein T is OR^{T1} with R^{T1} being different from hydrogen by standard etherification procedures, e.g. by reaction with an alkyl, alkenyl, alkynyl, aryl etc. halide R^{T1}-Hal (Hal=halide). Compounds (I.C) wherein T is OR^{T1} with R^{T1} being different from hydrogen can also be prepared in analogy to the syntheses outlined in schemes 9 and 10 by using instead of the alcohols (X1) or (XII) the etherified compounds (NH₂—C(R^{3c}R^{3d})—C(R^{3c}R^{3d})—OR^{T1} and S═C═N—C(R^{3c}R^{3d})—(R^{3c}R^{3d})—OR^{T1}, respectively.

Compounds (I.C) wherein T is SR^{T2} can also be prepared in analogy to the syntheses outlined in schemes 9 and 10 by using instead of the alcohols (XI) or (XII) the corresponding thiols (NH₂—C(R^{3c}R^{3d})—C(R^{3c}R^{3d})—SH and S═C═N—C(R^{3c}R^{3d})—C(R^{3c}R^{3d})—SH, respectively) or the corresponding thioethers (NH₂—C(R^{3c}R^{3d})—C(R^{3c}R^{3d})—SR^{T2} and S═C═N—C(R^{3c}R^{3d})—C(R^{3c}R^{3d})—SR^{T2}, respectively).

Compounds (III) and (IV) can also be converted into compounds (I.C) wherein T is —O—C(O)—R^{T3} or —O—C(S)—R^{T4} by esterification with the corresponding (thio)acid chloride Cl—C(V)—R^{T}, wherein V is O or S and R^{t} is R^{T3} if V is 0 and R^{T4} if V is S, as outlined below in scheme 11. X corresponds here to Y in the general formula (I.C).

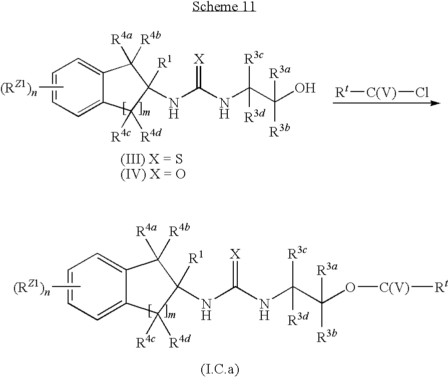

The variables R^{Z1}, R¹, R^{3a}, R^{3b}, R^{3c}, R^{3d}, R^{4a}, R^{4b}, R^{4c}, R^{4d}, m and n are as defined above. X is O or S, V is O or S, R^{t} is R^{T3} if V is 0 and R^{T4} if V is S.

The reaction is generally carried out in the presence of a base such as an amine. The (thio)acid chlorides are either commercially available or can be prepared from the corresponding acids or esters by chlorination e.g. with thionylchloride. The corresponding acids and esters can be prepared by standard procedures.

Compounds (I.C) wherein Y is O or S and T is —O—C(O)—R^{T3}, —O—C(S)—R^{T4} or S—C(S)—R^{T5} can also be prepared starting from an amine (V) and an iso(thio)cyanate (XIII) as shown in scheme 12.

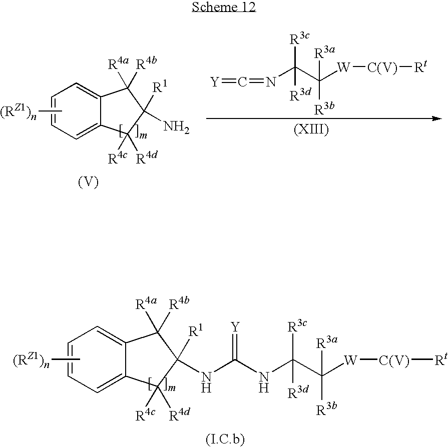

The variables R^{Z1}, R¹, R^{3a}, R^{3b}, R^{3c}, R^{3d}, R^{4a}, R^{4b}, R^{4c}, R^{4d}, m and n are as defined above. Y is O or S, V is O or S, R^{t} is R^{T3} if V and W are both O, R^{t} is R^{T4} if V is S and W is O, and R^{t} is R^{T5} if V and W are both S.

An amine (V) or a salt thereof can be converted to the corresponding (thio)urea compound (I.C.b) by reaction of the amine (V) with an iso(thio)cyanate (XIII) (see G. Liu et al. J. Org. Chem. 1999, 64, 1278-1284). Isothiocyanates of formula (XIII) can be prepared according to the procedures described in Coll. Czech. Chem. Commun. 1986, 51, 112-117.

Compounds of formula (I.C) wherein Y is NH can be synthesized in analogy to the method described in scheme 9, however using in the first step guanidine dichloride (Cl₂C═NH) instead of (thio)phosgene.

Alternatively, compounds of formula (I.C) wherein Y is NH can be synthesized as outlined in scheme 13 by reacting an amine (V) with the guanidine derivative (XIV):

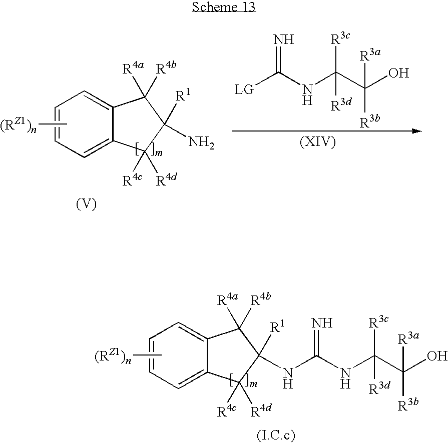

The variables R^{Z1}, R¹, R^{3a}, R^{3b}, R^{3c}, R^{3d}, R^{4a}, R^{4b}, R^{4c}, R^{4d}, m and n are as defined above. LG is Cl, imidazolyl or pyrazolyl.

Compounds of formula (I.C) wherein Y is NH can also be synthesized as outlined in scheme 14 by reacting an amine (V) with the guanidine derivative (XV) and subsequent alkylation of the NH₂ group:

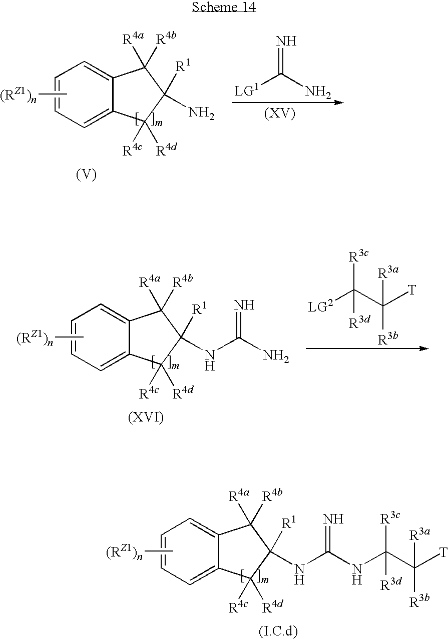

The variables R^{Z1}, R¹, R^{3a}, R^{3b}, R^{3c}, R^{3d}, R^{4a}, R^{4b}, R^{4c}, R^{4d}, m and n are as defined above. LG¹ is Cl, imidazolyl or pyrazolyl. LG¹ is a leaving group such as Cl, Br, a tosylate or a triflate group.

In a first step, an amine (V) is reacted with guanidine chloride or with imidazol or pyrazolamidine (XV) to the guanidine (XVI) which is then alkylated to give the compound (I.C.d). The alkylation can additionally take place at the NH groups of (XVI), thus providing compounds (I.C) wherein R²⁰ and/or Red and/or Rya are a group —C(R^{3c}R^{3d})—C(R^{3a}R^{3b})-T.

As a rule, the compounds of the formula (I) can be prepared by the methods described above. If individual compounds cannot be prepared via the above-described routes, they can be prepared by derivatization of other compounds (I) or by customary modifications of the synthesis routes described. For example, in individual cases, certain compounds (I) can advantageously be prepared from other compounds (I) by ester hydrolysis, amidation, esterification, ether cleavage, olefination, reduction, oxidation and the like.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or digestion.

Due to their excellent activity, the compounds of the general formula (I) may be used for controlling animal pests.

Accordingly, the present invention also provides a method for controlling animal pests which method comprises treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of a compound of the formula I or a salt thereof or a composition as defined above.

Preferably, the method of the invention serves for protecting seed and the plant which grows therefrom from animal pest attack or infestation and comprises treating the seed with a pesticidally effective amount of a compound of the formula I or an agriculturally acceptable salt thereof as defined above or with a pesticidally effective amount of an agricultural composition as defined above and below. The method of the invention is not limited to the protection of the “substrate” (plant, seed, soil material etc.) which has been treated according to the invention, but also has a preventive effect, thus, for example, according protection to a plant which grows from a treated seed, the plant itself not having been treated.

In the sense of the present incention, “animal pests” are preferably selected from arthropods and nematodes, more preferably from harmful insects, arachnids and nematodes, and even more preferably from insects, acarids and nematodes.

The invention further provides an agricultural composition for combating such animal pests, which comprises such an amount of at least one compound of the general formula (I) or at least one agriculturally useful salt thereof and at least one inert liquid and/or solid agronomically acceptable carrier that has a pesticidal action and, if desired, at least one surfactant.

Such a composition may contain a single active compound of the formula (I) or a salt thereof or a mixture of several active compounds (I) or their salts according to the present invention. The composition according to the present invention may comprise an individual isomer or mixtures of isomers as well as individual tautomers or mixtures of tautomers.

The compounds of the formula (I) and the pestidicidal compositions comprising them are effective agents for controlling arthropod pests and nematodes. Animal pests controlled by the compounds of formula (I) include for example

Insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lambdina fiscellaria, Laphygma exigua, Leucoptera coffeella, Leucoptera scitella, Lithocolletis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12-punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria;
dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;
thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;
hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;
heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;
homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla pin, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;
termites (Isoptera), e.g. Calotermes flavicollis, Leucotermes flavipes, Reticulitermes flavipes, Reticulitermes lucifugus and Termes natalensis;
orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;
Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidae spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;
Siphonatera, e.g. Xenopsylla cheopsis, Ceratophyllus spp;

The compositions and compounds of formula (I) are useful for the control of nematodes, especially plant parasitic nematodes such as root knot nematodes, Meloidogyne hapla, Meloidogyne incognita, Meloidogyne javanica, and other Meloidogyne species;

cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

In a preferred embodiment of the invention the compounds of formula (I) are used for controlling insects or arachnids, in particular insects of the orders Lepidoptera, Coleoptera, Thysanoptera and Homoptera and arachnids of the order Acarina. The compounds of the formula (I) according to the present invention are particularly useful for controlling insects of the order Thysanoptera and Homoptera.

The compounds of formula (I) or the pesticidal compositions comprising them may be used to protect growing plants and crops from attack or infestation by animal pests, especially insects, acaridae or arachnids by contacting the plant/crop with a pesticidally effective amount of compounds of formula (I). The term “crop” refers both to growing and harvested crops.

The compounds of formula (I) can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methyl-pyrrolidones [NMP], N-octyl-pyrrolidone [NOP]), acetates (glycol diacetate), glycols, fatty acid dimethylamides, fatty acids and fatty acid esters. In principle, solvent mixtures may also be used.

Suitable emulsifiers are nonionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

A suitable preservative is e.g. dichlorophen.

Seed treatment formulations may additionally comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of formula (I) can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:
1. Products for dilution with water for foliar applications. For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

### H) Gel-Formulation (GF)

In an agitated ball mill, 20 parts by weight of the active compound(s) are comminuted with addition of 10 parts by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active compound(s) is obtained.
2. Products to be applied undiluted for foliar applications. For seed treatment purposes, such products may be applied to the seed diluted or undiluted.

### I) Dustable Powders (DP, DS)

5 parts by weight of the active compound(s) are ground finely and mixed intimately with 95 parts by weight of finely divided kaolin. This gives a dustable product having 5% (w/w) of active compound(s)

### J) Granules (GR, FG, GG, MG)

0.5 parts by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

### K) ULV Solutions (UL)

10 parts by weight of the active compound(s) are dissolved in 90 parts by weight of an organic solvent, for example xylene. This gives a product having 10% (w/w) of active compound(s), which is applied undiluted for foliar use.

The compounds of formula (I) are also suitable for the treatment of seeds. Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Other preferred FS formulations of compounds of formula (I) for seed treatment comprise from 0.5 to 80 wt % of the active ingredient, from 0.05 to 5 wt % of a wetter, from 0.5 to 15 wt % of a dispersing agent, from 0.1 to 5 wt % of a thickener, from 5 to 20 wt % of an anti-freeze agent, from 0.1 to 2 wt % of an anti-foam agent, from 1 to 20 wt % of a pigment and/or a dye, from 0 to 15 wt % of a sticker/adhesion agent, from 0 to 75 wt % of a filler/vehicle, and from 0.01 to 1 wt % of a preservative.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula (I) are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula (I) are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickyness, moisture retention or aging characteristics.

The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula (I) as aerosols (e.g. in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula (I) and their respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of formula (I) and its respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-(+)trans-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculate, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons, such as styrene, and aliphatic diens, such as butadiene.

The impregnation of curtains and bednets is done in general by dipping the textile material into emulsions or dispersions of the insecticide or spraying them onto the nets.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of formula (I) into contact with each other, and “seed dressing” to methods of seed treatment which provide the seeds with an amount of the compounds of formula (I), i.e. which generate a seed comprising the compound of formula (I). In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of formula (I) or the enantiomers or veterinarily acceptable salts thereof are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula (I) or the enantiomers or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula (I) or the enantiomers or veterinarily acceptable salts thereof or a composition comprising it.

The invention also provides a process for the preparation of a composition for treating, controlling, preventing or protecting animals against infestation or infection by parasites which comprises a parasiticidally effective amount of a compound of formula (I) or the enantiomers or veterinarily acceptable salts thereof or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly, it has now been found that compounds of formula (I) are suitable for combating endo- and ectoparasites in and on animals.

Compounds of formula (I) or the enantiomers or veterinarily acceptable salts thereof and compositions comprising them are preferably used for controlling and preventing infestations and infections in animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula (I) or the enantiomers or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula (I) or the enantiomers or veterinarily acceptable salts thereof and compositions comprising them are suitable for systemic and/or non-systemic control of ecto- and/or endoparasites. They are active against all or some stages of development.

The compounds of formula (I) are especially useful for combating ectoparasites.

The compounds of formula (I) are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,
cockroaches (Blattaria—Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles mini-mus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,
Actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
Bugs (Heteropterida): Cimex lectularius, Cimex hemipterus, Reduvius senilis, Triatoma spp., Rhodnius ssp., Panstrongylus ssp. and Arilus critatus,
Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp,
Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,
Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula (I) and compositions containing them are particularly useful for the control of pests from the orders Diptera, Siphonaptera and Ixodida.

Moreover, the use of the formula (I) and compositions containing them for combating mosquitoes is especially preferred.

The use of the compounds of formula (I) and compositions containing them for combating flies is a further preferred embodiment of the present invention.

Furthermore, the use of the compounds of formula (I) and compositions containing them for combating fleas is especially preferred.

The use of the compounds of formula formula (I) and compositions containing them for combating ticks is a further preferred embodiment of the present invention.

The compounds of formula (I) also are especially useful for combating endoparasites (roundworms nematoda, thorny headed worms and planarians).

Administration can be carried out both prophylactically and therapeutically.

Administration of the active compounds is carried out directly or in the form of suitable preparations, orally, topically/dermally or parenterally.

For oral administration to warm-blooded animals, the formula (I) compounds may be formulated as animal feeds, animal feed premixes, animal feed concentrates, pills, solutions, pastes, suspensions, drenches, gels, tablets, boluses and capsules. In addition, the formula (I) compounds may be administered to the animals in their drinking water. For oral administration, the dosage form chosen should provide the animal with 0.01 mg/kg to 100 mg/kg of animal body weight per day of the formula (I) compound, preferably with 0.5 mg/kg to 100 mg/kg of animal body weight per day.

Alternatively, the formula (I) compounds may be administered to animals parenterally, for example, by intraruminal, intramuscular, intravenous or subcutaneous injection. The formula (I) compounds may be dispersed or dissolved in a physiologically acceptable carrier for subcutaneous injection. Alternatively, the formula (I) compounds may be formulated into an implant for subcutaneous administration. In addition the formula (I) compound may be transdermally administered to animals. For parenteral administration, the dosage form chosen should provide the animal with 0.01 mg/kg to 100 mg/kg of animal body weight per day of the formula (I) compound.

The formula (I) compounds may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the formula (I) compound. In addition, the formula (I) compounds may be formulated as ear tags for animals, particularly quadrupeds such as cattle and sheep.

Suitable preparations are:
Solutions such as oral solutions, concentrates for oral administration after dilution, solutions for use on the skin or in body cavities, pouring-on formulations, gels;
Emulsions and suspensions for oral or dermal administration; semi-solid preparations;
Formulations in which the active compound is processed in an ointment base or in an oil-in-water or water-in-oil emulsion base;
Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methyl-pyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminum monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointment-like consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N-methylpyrrolidone, 2-pyrrolidone, 2,2-dimethyl-4-oxy-methylene-1,3-diox-olane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter,
fatty alcohols such as isotridecyl alcohol, 2-octyldodecanol, cetylstearyl alcohol, oleyl alcohol, and
fatty acids such as oleic acid and
mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether;
ampholytic surfactants such as di-sodium N-lauryl-p-iminodipropionate or lecithin;
anionic surfactants, such as sodium lauryl sulfate, fatty alcohol ether sulfates, mono/dialkyl polyglycol ether orthophosphoric acid ester monoethanolamine salt;
cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminum oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compound of formula (I).

Generally, it is favorable to apply the compounds of formula (I) in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula (I) against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula (I) are applied dermally/topically.

In a further preferred embodiment, the topical application is conducted in the form of compound-containing shaped articles such as collars, medallions, ear tags, bands for fixing at body parts, and adhesive strips and foils.

Generally, it is favorable to apply solid formulations which release compounds of formula (I) in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula (I). A detailed list of plastics and elastomers as well as preparation procedures for the shaped articles is given e.g. in WO 03/086075.

Compositions to be used according to this invention may also contain other active ingredients, for example other pesticides, insecticides, herbicides, fungicides, other pesticides, or bactericides, fertilizers such as ammonium nitrate, urea, potash, and superphosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents used according to the invention in a weight ratio of 1:10 to 10:1. Mixing the compounds (I) or the compositions comprising them in the use form as pesticides with other pesticides frequently results in a broader pesticidal spectrum of action.

The following list of pesticides together with which the compounds (I) can be used, is intended to illustrate the possible combinations, but not to impose any limitation:

A.1. Organo(thio)phosphates: acephate, azamethiphos, azinphos-ethyl, azinphosmethyl, chlorethoxyfos, chlorfenvinphos, chlormephos, chlorpyrifos, chlorpyrifosmethyl, coumaphos, cyanophos, demeton-5-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
A.2. Carbamates: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
A.3. Pyrethroids: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-, yfluthrin, cyhalothrin, lambda-cyhalothrin, gamma-cyhalothrin, cypermethrin, alphacypermethrin, beta-cypermethrin, theta-cypermethrin, zeta-cypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, permethrin, phenothrin, prallethrin, resmethrin, RU 15525, silafluofen, tefluthrin, tetramethrin, tralomethrin, transfluthrin, ZXI 8901;
A.4. Juvenile hormone mimics: hydroprene, kinoprene, methoprene, fenoxycarb, pyriproxyfen;
A.5. Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), thiacloprid, thiocyclam, thiosultap-sodium, and AKD1022.
A.6. GABA gated chloride channel antagonist compounds: chlordane, endosulfan, gamma-HCH (lindane); acetoprole, ethiprole, fipronil, pyrafluprole, pyriprole, vaniliprole, the phenylpyrazole compound of formula Γ¹

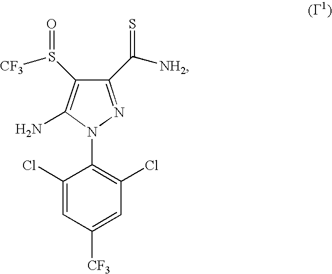

A.7. Chloride channel activators: abamectin, emamectin benzoate, milbemectin, lepimectin;
A.8. METI I compounds: fenazaquin, fenpyroximate, pyrimidifen, pyridaben, tebufenpyrad, tolfenpyrad, flufenerim, rotenone;
A.9. METI II and III compounds: acequinocyl, fluacyprim, hydramethylnon;
A.10. Uncouplers of oxidative phosphorylation: chlorfenapyr, DNOC;
A.11. Inhibitors of oxidative phosphorylation: azocyclotin, cyhexatin, diafenthiuron, fenbutatin oxide, propargite, tetradifon;
A.12. Moulting disruptors: cyromazine, chromafenozide, halofenozide, methoxyfenozide, tebufenozide;
A.13. Synergists: piperonyl butoxide, tribufos;
A.14. Sodium channel blocker compounds: indoxacarb, metaflumizone;
A.15. Fumigants: methyl bromide, chloropicrin sulfuryl fluoride;
A.16. Selective feeding blockers: crylotie, pymetrozine, flonicamid;
A.17. Mite growth inhibitors: clofentezine, hexythiazox, etoxazole;
A.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;
A.19. Lipid biosynthesis inhibitors: spirodiclofen, spiromesifen, spirotetramat;
A.20. octapaminergic agonsits: amitraz;
A.21. ryanodine receptor modulators: flubendiamide;
A.22. Various: aluminum phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, tartar emetic;
A.23. N—R′-2,2-dihalo-1-R″cyclo-propanecarboxamide-2-(2,6-dichloro-α,α,α-tri-fluoro-p-tolyl)hydrazone or N—R′-2,2-di(R′″)propionamide-2-(2,6-dichloro-α,α,α-trifluoro-p-tolyl)hydrazone, wherein R′ is methyl or ethyl, halo is chloro or bromo, R″ is hydrogen or methyl and R′″ is methyl or ethyl;
A.24. Anthranilamides: chloranthraniliprole, the compound of formula Γ²

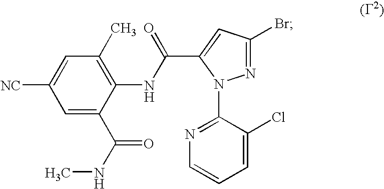

A.25. Malononitrile compounds: CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN)₂CH₂CH₂CF₂CF₃;
A.26. Microbial disruptors: Bacillus thuringiensis subsp. lsraelensi, Bacillus sphaericus, Bacillus thuringiensis subsp. Aizawai, Bacillus thuringiensis subsp. Kurstaki, Bacillus thuringiensis subsp. Tenebrionis;
A.27. Alkynylether compounds Γ⁴ and Γ⁵:

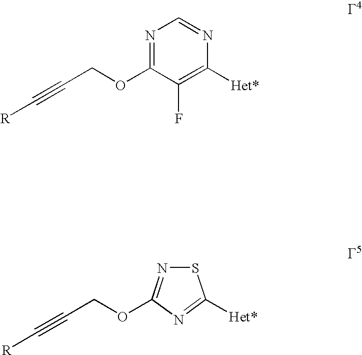

wherein R is methyl or ethyl and Het* is 3,3-dimethylpyrrolidin-1-yl, 3-methylpiperidin-1-yl, 3,5-dimethylpiperidin-1-yl, 3-trifluormethylpiperidin-1-yl, hexahydroazepin-1-yl, 2,6-dimethylhexahydroazepin-1-yl or 2,6-dimethylmorpholin-4-yl. These compounds are described e.g. in JP 2006131529.

The commercially available compounds of the group A may be found in The Pesticide Manual, 13th Edition, British Crop Protection Council (2003) among other publications.

Thioamides of formula Γ¹ and their preparation have been described in WO 98/28279. Lepimectin is known from Agro Project, PJB Publications Ltd, November 2004. Benclothiaz and its preparation have been described in EP-A1454621. Methidathion and Paraoxon and their preparation have been described in Farm Chemicals Handbook, Volume 88, Meister Publishing Company, 2001. Acetoprole and its preparation have been described in WO 98/28277. Metaflumizone and its preparation have been described in EP-A1462 456. Flupyrazofos has been described in Pesticide Science 54, 1988, p. 237-243 and in U.S. Pat. No. 4,822,779. Pyrafluprole and its preparation have been described in JP 2002193709 and in WO 01/00614. Pyriprole and its preparation have been described in WO 98/45274 and in U.S. Pat. No. 6,335,357. Amidoflumet and its preparation have been described in U.S. Pat. No. 6,221,890 and in JP 21010907. Flufenerim and its preparation have been described in WO 03/007717 and in WO 03/007718. AKD 1022 and its preparation have been described in U.S. Pat. No. 6,300,348. Chloranthraniliprole has been described in WO 01/70671, WO 03/015519 and WO 05/118552. Anthranilamide derivatives of formula Γ² have been described in WO 01/70671, WO 04/067528 and WO 05/118552. Cyflumetofen and its preparation have been described in WO 04/080180. The aminoquinazolinone compound pyrifluquinazon has been described in EP A 109 7932. The malononitrile compounds CF₃(CH₂)₂C(CN)₂CH₂(CF₂)₃CF₂H, CF₃(CH₂)₂C(CN)₂CH₂(CF₂)₅CF₂H, CF₃(CH₂)₂C(CN)₂(CH₂)₂C(CF₃)₂F, CF₃(CH₂)₂C(CN)₂(CH₂)₂(CF₂)₃CF₃, CF₂H(CF₂)₃CH₂C(CN)₂CH₂(CF₂)₃CF₂H, CF₃(CH₂)₂C(CN)₂CH₂(CF₂)₃CF₃, CF₃(CF₂)₂CH₂C(CN)₂CH₂(CF₂)₃CF₂H, CF₃CF₂CH₂C(CN)₂CH₂(CF₂)₃CF₂H, 2-(2,2,3,3,4,4,5,5-octafluoropentyl)-2-(3,3,4,4,4-pentafluorobutyl)-malonodinitrile, and CF₂HCF₂CF₂CF₂CH₂C(CN) 2CH₂CH₂CF₂CF₃ have been described in WO 05/63694.

Fungicidal mixing partners are those selected from the group consisting of acylalanines such as benalaxyl, metalaxyl, ofurace, oxadixyl,

amine derivatives such as aldimorph, dodine, dodemorph, fenpropimorph, fenpropidin, guazatine, iminoctadine, spiroxamin, tridemorph,
anilinopyrimidines such as pyrimethanil, mepanipyrim or cyrodinyl,
antibiotics such as cycloheximid, griseofulvin, kasugamycin, natamycin, polyoxin or streptomycin,
azoles such as bitertanol, bromoconazole, cyproconazole, difenoconazole, diniconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol,
dicarboximides such as iprodion, myclozolin, procymidon, vinclozolin, dithiocarbamates such as ferbam, nabam, maneb, mancozeb, metam, metiram, propineb, polycarbamate, thiram, ziram, zineb,
heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine,
copper fungicides such as Bordeaux mixture, copper acetate, copper oxychloride, basic copper sulfate,
nitrophenyl derivatives such as binapacryl, dinocap, dinobuton, nitrophthalisopropyl,
phenylpyrroles such as fenpiclonil or fludioxonil, sulfur,
other fungicides such as acibenzolar-5-methyl, benthiavalicarb, carpropamid, chlorothalonil, cyflufenamid, cymoxanil, diclomezin, diclocymet, diethofencarb, edifenphos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid,
strobilurins such as azoxystrobin, dimoxystrobin, fluoxastrobin, kresoxim-methyl, metominostrobin, orysastrobin, picoxystrobin or trifloxystrobin,
sulfenic acid derivatives such as captafol, captan, dichlofluanid, folpet, tolylfluanid, cinnemamides and analogs such as dimethomorph, flumetover or flumorph.

The animal pest, i.e. arthropodes and nematodes, the plant, soil or water in which the plant is growing can be contacted with the present compound(s) (I) or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

Moreover, animal pests may be controlled by contacting the target pest, its food supply, habitat, breeding ground or its locus with a pesticidally effective amount of compounds of formula (I). As such, the application may be carried out before or after the infection of the locus, growing crops, or harvested crops by the pest.

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula (I) and its compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of the invention can also be applied preventively to places at which occurrence of the pests is expected.

The compounds of formula (I) may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95% by weight, preferably from 0.1 to 45% by weight, and more preferably from 1 to 25% by weight of at least one repellent and/or insecticide.

For use in bait compositions, the typical content of active ingredient is from 0.001% by weight to 15% by weight, desirably from 0.001% by weight to 5% by weight of active compound.

For use in spray compositions, the content of active ingredient is from 0.001 to 80% by weight, preferably from 0.01 to 50% by weight and most preferably from 0.01 to 15% by weight.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the mixture are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

### PREPARATION EXAMPLES

### I. Compounds of the formula (I) wherein Z is a group of the formula II.A or II.B

### Example 1

### (4,5-Dihydro-thiazol-2-yl)-(7-methoxy-1,2,3,4-tetrahydro-naphthalen-2-yl)-amine (compound 1)

To a solution of 1-(2-hydroxy-ethyl)-3-(7-methoxy-1,2,3,4-tetrahydronaphthalen-2-yl)thiourea (0.28 g) and diisopropyethylamine (0.18 g) in propionitrile (15 mL) was added cyanomethyltrimethylphosphonium iodide (0.30 g, Tetrahedron 2001, 57, 5451-54). The reaction mixture was heated up to 90° C. for 5 h and stirring was continued overnight at room temperature. Purification by chromatography on silica gel yielded 0.11 g of the title compound.

### Example 2

### (8-Chloro-1,2,3,4-tetrahydro-naphthalen-2-yl)-(4,5-dihydro-thiazol-2-yl)-amine (compound 2a) and (8-chloro-1,2,3,4-tetrahydro-naphthalen-2-yl)-(4,5,4′,5′-tetrahydro-[2,3′]bithiazolyl-2′ylidene)amine (compound 2b)

A solution of 8-chloro-1,2,3,4-tetrahydro-naphthalen-2-ylamine (430 mg, 2.37 mmol) in diethyl ether (10 mL) was cooled to 0° C. Then a solution of 2-chloroethylisothiocyanate (0.23 mL, 2.37 mmol) in diethyl ether (10 mL) was added dropwise and the reaction mixture stirred for 3 h. After addition of aq. NaOH-solution (1 M, 4 mL) and water (15 mL), the phases were separated. The aq. phase was extracted with diethyl ether (3 times) and the combined organic phases washed with water (3 times) and dried over Na₂SO₄. Purification by flash chromatography (SiO₂, CH₂Cl₂/methanol 100:0→90:10) yielded (8-chloro-1,2,3,4-tetrahydro-naphthalen-2-yl)(4,5-dihydro-thiazol-2-yl)-amine (340 mg, 1.27 mmol, 54%) and (8-chloro-1,2,3,4-tetrahydro-naphthalen-2-yl)-(4,5,4′,5′-tetrahydro-[2,3′]bithiazolyl-2′-ylidene)amine (140 mg, 0.40 mmol, 17%).

Compounds of the formula (I) (wherein R¹, R^{3a}, R^{3b}, R^{3b}, R^{3c}, R^{3d}, R^{4a}, R^{4b}, R^{4c} and R^{4d} are hydrogen) listed in table I below were prepared in an analogous manner.

TABLE I

| | |
|---|---|
| 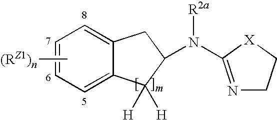 | 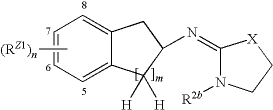 |

| | | | | | | Physico-chemical data |
|---|---|---|---|---|---|---|
| | | | | | | m.p. [° C.]/¹H-NMR |
| Comp. | (R^{Z1})ₙ | m | R^{2a} | R^{2b} | X | (CDCl₃) δ ppm/r.t.; M+ |
| 1 | 7-OCH₃ | 2 | H | — | S | 1.7-2.15 (m), 2.7-2.9 (m), |
| | | | | | | 3.15 (mc), 3.6 (mc), 3.75 |
| | | | | | | (mc), 4.45 (br s ), 6.6 (s), |
| | | | | | | 6.7 (d), 7.0 (d) |
| 2a | 8 Cl | 2 | H | | S | 180-184 |
| 2b | 8 Cl | 2 | | 4,5-dihydrothiazol-2-yl | S | 92-94 |
| 3a | n = 0 | 2 | H | | S | 136-138 |
| 3b | n = 0 | 2 | | 4,5-dihydrothiazol-2-yl | S | 146-148 |
| 4a | 5-OCH₃ | 2 | H | | S | 155-157 |
| 4b | 5-OCH₃ | 2 | | 4,5-dihydrothiazol-2-yl | S | 94.5-96.5 |
| 5a | 7 Cl | 2 | H | | S | 206-210 |
| 5b | 7 Cl | 2 | | 4,5-dihydrothiazol-2-yl | S | 2.91 min; 352 |
| 6a | 6 Cl | 2 | H | | S | 266-268 |
| 6b | 6 Cl | 2 | | 4,5-dihydrothiazol-2-yl | S | 126-130 |
| 7 | 6-Br | 2 | H | | S | 270-271 |
| 8a | 5-F | 2 | H | | S | 185-188 |
| 8b | 5-F | 2 | | 4,5-dihydrothiazol-2-yl | S | 162-164 |
| 9a | 6-OCH₃ | 2 | H | | S | 128-130 |
| 9b | 6-OCH₃ | 2 | | 4,5-dihydrothiazol-2-yl | S | 2.59 min; 348 |
| 10a | 5 Cl | 2 | H | | S | 180-185 |
| 10b | 5 Cl | 2 | | 4,5-dihydrothiazol-2-yl | S | 132-134 |
| 11 | n = 0 | 1 | H | | S | 161-162 |
| 12 | n = 0 | 1 | H | | O | 136-139 |
| 13 | n = 0 | 1 | H | | NH | |

comp. = compound number
m.p. = melting point
r.t. = retention time (H PLC)
M+ = molecular mass of the detected cationic species
s = singulet
d = doublet
m = multiplet
mc = centered multiplet
br = broad

### II. Compounds of the Formula (I) Wherein Z is a Group of the Formula (II.C)

### Example 14

### 1-(2-Hydroxyethyl)-3-(1,2,3,4-tetrahydronaphthalen-2-yl)-thiourea (compound 14)

A solution of thiophosgen (0.73 g) in chloroform (10 ml) was treated at room temperature with a solution of potassium carbonate (2.23 g) in water (15 mL) and a solution of 1,2,3,4-tetrahydro-naphthalen-2-ylamine hydrochloride (1.00 g) in chloroform (25 mL). After stirring overnight dichloromethane was added and the mixture extracted with water (3×), the organic phase dried over sodium sulphate and the solvent evaporated under reduced pressure to yield 0.83 g of the crude isothiocyanate. The latter was dissolved in toluene (20 ml) and treated with 2-aminoethanol at reflux for 4 h. Stirring was continued overnight at room temperature. The mixture was then dissolved in ethyl acetate and extracted with water (3×), the organic phase dried and evaporated in vacuo. The remainder was purified by column chromatography on silica gel to yield 1.00 g of the product, m.p. 89-91° C.

### Example 15

### 2-Acetoxy-2-methyl-propionic acid 2-[3-(1,2,3,4-tetrahydro-naphthalen-2-yl)thioureido]-ethyl ester (compound 15)

A solution of 1-(2-hydroxyethyl)-3-(1,2,3,4-tetrahydronaphthalen-2-yl)-thiourea (0.40 g) and triethylamine (0.16 g) in anhydrous tetrahydrofuran (20 mL) was treated with acetic acid 1-chlorocarbonyl-1-methylethyl ester (0.26 g) and stirred overnight. Purification by column chromatography on silica gel yielded 0.46 g of the product as a colourless oil.

### Example 16

### Benzoic acid 2-[3-(7-methoxy-1,2,3,4-tetrahydro-naphthalen-2-yl)-thioureido]-ethyl ester (compound 16)

A solution of 7-methoxy-1,2,3,4-tetrahydro-naphthalen-2-ylamine (0.34 g) in toluene (10 ml) was treated with benzoic acid 2-isothiocyanato-ethyl ester (0.39 g, cf. Collect. Czech. Chem. Commun. 1986, 51, 112-117), refluxed for 1 h and stirred at room temperature overnight. Purification by Chromatography on silica gel yielded the product (0.32 g), m.p. 103-106° C.

### Example 17

Acetic acid 2-[3-(7-methoxy-1,2,3,4-tetrahydro-naphthalen-2-yl)-thioureido]-ethyl ester (compound 17)

A solution of 7-methoxy-1,2,3,4-tetrahydro-naphthalen-2-ylamine (0.34 g) in toluene (10 mL) was treated with acetic acid 2-isothiocyanato-ethyl ester (0.28 g, cf. Collect. Czech. Chem. Commun. 1986, 51, 112-117), refluxed for 1 h and stirred at room temperature overnight. Purification by chromatography on silica gel yielded the product (0.45 g).

Compounds of the formula (I) (wherein R¹, R^{2c}, R^{2d}, R^{3a}, R^{3b}, R^{3b}, R^{3c}, R^{3d}, R^{4a}, R^{4b}, R^{4c} and R^{4d} are hydrogen and Y is sulfur) listed in table II below were prepared in an analogous manner.

TABLE II

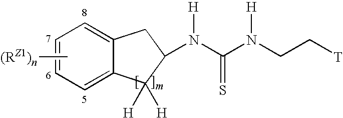

| | | | | Physico-chemical data |
|---|---|---|---|---|
| | | | | m.p. [° C.]/ |
| | | | | ¹H-NMR (CDCl₃) δ ppm/ |
| Comp | (R^{Z1})ₙ | m | T | r.t.; (M + H)⁺ |
| 14 | n = 0 | 2 | OH | 89-91 |
| 15 | n = 0 | 2 | —O—C(O)—C(CH₃)₂—O—C(O)—CH₃ | 1.55 (s), 1.8-2.2 (m), 2.75 |
| | | | | (mc), 2.9 (mc), 3.2 (mc), |
| | | | | 3.95 (mc), 4.45 (mc), 6.1 |
| | | | | (br s), 6.35 (br s), 7.1 (mc) |
| 16 | 7-OCH₃ | 2 | benzoyloxy | 103-106 |
| 17 | 7-OCH₃ | 2 | —O—C(O)—CH₃ | 1.6-2.2 (m), 2.7-2.9 (m), |
| | | | | 3.2 (mc), 3.75 (mc), 4.25 |
| | | | | (mc), 6.05 (br s), 6.2 (br |
| | | | | s), 6.6 (s), 6.75 (d), 7.0 (d) |
| 18 | 7-OCH₃ | 2 | —OH | 82-85 |
| 19 | 7-OCH₃ | 2 | —O—C(O)—CH₂—O—CH₃ | 1.7-2.15 (m), 2.7-3.5 (m), |
| | | | | 3.75 (s), 3.85 (mc), 4.05 |
| | | | | (s), 4.35 (mc), 6.1 (br s), |
| | | | | 6.2 (br s), 6.6 (s), 6.7 (d), |
| | | | | 7.0 (d) |
| 20 | 7-OCH₃ | 2 | O C(O) C(CH3)2 O C(O) CH₃ | 1.5-2.2 (m), 2.65-3.2 (m), |
| | | | | 3.75 (s), 3.95 (mc), 4.45 |
| | | | | (mc), 6.05 (br s), 6.25 (br |
| | | | | s), 6.6 (s), 6.7 (d), 7.0 (d) |
| 21 | 5-OCH₃ | 2 | OH | 122-125 |
| 22 | n = 0 | 2 | O C(O) CH₂ O CH₃ | 1.7-2.2 (m), 2.7-3.25 (m), |
| | | | | 3.45 (s), 3.85 (mc), 4.05 |
| | | | | (s), 4.35 (mc), 6.05 (br s), |
| | | | | 6.2 (br s), 7.0-7.2 (m) |
| 23 | 5-OCH₃ | 2 | O C(O) CH₂ O CH₃ | 1.85-2.15 (m), 2.75-2.85 |
| | | | | (m), 3.15 (mc), 3.4 (s), 3.8 |
| | | | | (s), 3.85 (mc), 4.05 (s), |
| | | | | 4.35 (mc), 6.05 (br s), 6.1 |
| | | | | (br s), 6.75 (mc), 7.1 (m) |
| 24 | 5-OCH₃ | 2 | O C(O) C(CH₃)₂ O C(O) CH₃ | 1.55 (s), 1.75-2.15 (m), |
| | | | | 2.65-2.85 (m), 3.15 (mc), |
| | | | | 3.8 (s), 3.95 (mc), 4.45 |
| | | | | (mc), 6.1 (br s), 6.35 (br |
| | | | | s), 6.65 (mc), 7.1 (mc) |
| 25 | n = 0 | 1 | OH | 119-121 |
| 26 | n = 0 | 1 | isoxazol-5-ylcarbonyloxy | 2.85 min; 332 |
| 27 | n = 0 | 1 | 5-bromopyridin-3- | 3.2 min; 421 |
| | | | ylcarbonyloxy | |
| 28 | n = 0 | 1 | 3-trifluorobenzoyloxy | 3.74 min; 409 |
| 29 | n = 0 | 1 | —O C(O) CH(CH₃)—O—Ph | 3.53 min; 385 |
| 30 | n = 0 | 1 | 2-ethylsulfanylpyridin-3- | 3.34 min; 402 |
| | | | ylcarbonyloxy | |
| 31 | n = 0 | 1 | 2-methylsulfanylpyridin-3- | 3.4 min; 388 |
| | | | ylcarbonyloxy | |
| 32 | n = 0 | 1 | benzylcarbonyloxy | 3.41 min; 355 |
| 33 | n = 0 | 1 | furan-2-ylcarbonyloxy | 3.05 min; 331 |
| 34 | n = 0 | 1 | 5-chlorothien-2-ylcarbonyloxy | 3.62 min; 381 |
| 35 | n = 0 | 1 | 2-acetoxyphenylcarbonyloxy | 3.31 min; 399 |
| 36 | n = 0 | 1 | —O C(O) CH(Ph)₂ | 3.86 min; 431 |
| 37 | n = 0 | 1 | 3-(2-chlorophenyl)-5-methyl- | 3.59 min; 456 |
| | | | isoxazol-4-yl-carbonyloxy | |
| 38 | n = 0 | 1 | O C(O) CH(Ph) CH₂ CH₃ | 3.75 min; 383 |
| 39 | n = 0 | 1 | cyclopentylcarbonyloxy | 3.45 min; 333 |
| 40 | n = 0 | 1 | —O C(O) CH₂ CH(CH₃)₂ | 3.4 min; 321 |
| 41 | n = 0 | 1 | naphthalene-2-carbonyloxy | 3.75 min; 391 |
| 42 | n = 0 | 1 | —O C(O) C(CH₃)₃ | 3.41 min; 321 |
| 43 | n = 0 | 1 | —O C(O) CH₂ CH₂—Ph | 3.57 min; 369 |
| 44 | n = 0 | 1 | thiophen-2-yl-acetoxy | 3.34 min; 360 |
| 45 | n = 0 | 1 | O C(O) CH₂ CH₃ | 2.97 min; 293 |
| 46 | n = 0 | 1 | —O C(O) CH₂—O—Ph | 3.38 min; 371 |
| 47 | n = 0 | 1 | —O C(O) CH₂ CH₂—SCH₃ | 3.12 min; 339 |
| 48 | n = 0 | 1 | 5-ethoxycarbonyl-1-methyl-1H- | 3.31 min; 417 |
| | | | pyrazole-4-carbonyloxy | |
| 49 | n = 0 | 1 | O C(O) CH₂ O CH₃ | 2.68 min; 309 |
| 50 | n = 0 | 1 | 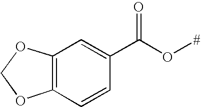 | 3.33 min; 385 |
| 51 | n = 0 | 1 | 6-chloropyridin-3-ylcarbonyloxy | 3.26 min; 376 |
| 52 | n = 0 | 1 | O C(O) C(CH₃)₂ O C(O) CH₃ | 3.19 min; 365 |
| 53 | n = 0 | 1 | 2,5-dimethylfuran-3- | 3.5 min; 359 |
| | | | ylcarbonyloxy | |

comp. = compound
m.p. = melting point
r.t. = retention time (H PLC)
(M + H)⁺= molecular mass of the detected cationic species
Ph = phenyl
# is the binding site to the remainder of the molecule
s = singlet
d = doublet
m = multiplet
mc = centered multiplet
br = broad

2. Biological Examples
   II.1 Cotton Aphid (Aphis gossypii), Mixed Life Stages

The active compounds were formulated in 50:50 acetone: water and 100 ppm Kinetic® surfactant.

Cotton plants at the cotyledon stage (one plant per pot) were infested by placing a heavily infested leaf from the main colony on top of each cotyledon. The aphids were allowed to transfer to the host plant overnight, and the leaf used to transfer the aphids was removed. The cotyledons were dipped in the test solution and allowed to dry. After 5 days, mortality counts were made.

In this test, the compounds 2a, 3a, 8b, 9a, 11, 12, 18, 22, 24, 25, 26, 27, 28, 30, 31, 32, 33, 34, 35, 36, 39, 40, 43, 44, 45, 46, 47, 48, 49, 51 and 52 at 300 ppm showed a mortality of at least 50% in comparison with untreated controls.

II.2 Green Peach Aphid (Myzus persicae), Mixed Life Stages

The active compounds were formulated in 50:50 acetone:water and 100 ppm Kinetic® surfactant.

Pepper plants in the 2^{nd} leaf-pair stage (variety ‘California Wonder’) were infested with approximately 40 laboratory-reared aphids by placing infested leaf sections on top of the test plants. The leaf sections were removed after 24 hr. The leaves of the intact plants were dipped into gradient solutions of the test compound and allowed to dry. Test plants were maintained under fluorescent light (24 hour photoperiod) at about 25° C. and 20-40% relative humidity. Aphid mortality on the treated plants, relative to mortality on check plants, was determined after 5 days.

In this test, compounds 3a, 4a, 11, 12, 18, 25, 26, 27, 28, 30, 31, 32, 33, 34, 35, 36, 38, 39, 40, 43, 44, 45, 46, 47, 48, 49 and 52 at 300 ppm showed a mortality of at least 50% in comparison with untreated controls.

II.3 Cowpea Aphid (Aphis craccivora)

The active compounds were formulated in 50:50 acetone:water and 0.1% (vol/vol) Alkamuls EL 620 surfactant.

Potted cowpea beans of 7-10 days of age are inoculated with aphids 24 h before treatment by clipping a leaf infested with cowpea aphid approximately 30 individuals. The treated beans are sprayed with 5 ml of the test solution using air driven hand atomizer (Devillbis atomizer) at 25 psi, allowed to air dry and kept at 25-27° C. and 50-60% humidity for 3 days. After 72 h, mortality was assessed.

In this test, the compounds 3a, 4b, 5a, 11, 12, 18, 25, 26, 29, 30, 34, 35, 38, 39, 40, 43, 44, 45 and 47 at 300 ppm showed a mortality of at least 60% in comparison with untreated controls.

II.4 Silverleaf Whitefly (Bemisia argentifolii), Adult

The active compounds were formulated in 50:50 acetone:water and 100 ppm Kinetic® surfactant.

Selected cotton plants were grown to the cotyledon state (one plant per pot). The cotyledons were dipped into the test solution to provide complete coverage of the foliage and placed in a well-vented area to dry. Each pot with treated seedling was placed in a plastic cup and 10 to 12 whitefly adults (approximately 3-5 day old) were introduced. The insects were collected using an aspirator and an 0.6 cm, non-toxic Tygon® tubing (R-3603) connected to a barrier pipette tip. The tip, containing the collected insects, was then gently inserted into the soil containing the treated plant, allowing insects to crawl out of the tip to reach the foliage for feeding. The cups were covered with a reusable screened lid (150 micron mesh polyester screen PeCap from Tetko Inc). Test plants were maintained in the holding room at about 25° C. and 20-40% relative humidity for 3 days avoiding direct exposure to the fluorescent light (24 hour photoperiod) to prevent trapping of heat inside the cup. Mortality was assessed 3 days after treatment of the plants.

In this test, compounds 3a, 3b, 4a, 16, 17, 18 and 40, at 300 ppm showed a mortality of at least 50% in comparison with untreated controls.

II.5 Brown Planthopper (Nilaparvata lugens)

The active compounds were formulated as a 20:80 acetone:water solution. Surfactant (Alkamuls EL 620) was added at the rate of 0.1% (vol/vol).

Potted rice plants of 3-4 weeks of age are sprayed with 10 ml of the test solution using air driven hand atomizer (Devillbis atomizer) at 1.7 bar. The treated plants are allowed to dry for about 1 hour and covered with Mylar cages. The plants are inoculated with 10 adults of the specie (5 male and 5 females) and kept at 25-27° C. and 50-60% humidity for 3 days. Mortality is assed after 24, 48 and 72 hours after treatment. Dead insects are usually found in the water surface. Each treatment is replicated once.

In this test, compounds 4b, 12, 17 and 19 at 300 ppm showed a mortality of at least 50% in comparison with untreated controls.

II.6 Southern Armyworm (Spodoptera eridania), 2^{nd}-3^{rd} Instar Larvae

The active compounds were formulated as a 10.000 ppm solution in a mixture of 35% acetone and water, which was diluted with water, if needed.

A Sieva lima bean leaf expanded to 7-8 cm in length is dipped in the test solution with agitation for 3 seconds and allowed to dry in a hood. The leaf is then placed in a 100×10 mm petri dish containing a damp filter paper on the bottom and ten 2nd instar caterpillars. At 4 days, observations are made of mortality, reduced feeding, or any interference with normal molting.

In this test, compounds 4a, 4b, 23, 32 and 49 at 300 ppm showed a mortality of at least 50% in comparison with untreated controls.

II.7 Activity Against Vetch Aphid (Megoura viciae)

The active compounds were formulated in DMSO:water (1:3). Bean leaf disks were placed into microtiterplates filled with 0.8% agar-agar and 2.5 ppm OPUS™. The leaf disks were sprayed with 2.5 μl of the test solution and 5 to 8 adult aphids were placed into the microtiterplates which were then closed and kept at 22-24° C. and 35-45% under fluorescent light for 6 days. Mortality was assessed on the basis of vital, reproduced aphids. Tests were replicated 2 times.

In this test, the compounds 8a, 9a, 9b, 18, 33, 39, 45, 47, 49 and 53 at a concentration of the test solution of 2500 mg/L showed a mortality of at least 50%.

II.8 Activity Against Boll Weevil (Anthonomus grandis)

The active compounds were formulated in DMSO:water (1:3). 10 to 15 eggs were placed into microtiterplates filled with 2% agar-agar in water and 300 ppm formaline. The eggs were sprayed with 20 μl of the test solution, the plates were sealed with pierced foils and kept at 24-26° C. and 75-85% humidity with a day/night cycle for 3 to 5 days. Mortality was assessed on the basis of the remaining unhatched eggs or larvae on the agar surface and/or quantity and depth of the digging channels caused by the hatched larvae. Tests were replicated 2 times.

In this test, the compounds 4a, 5a, 25 and 40, at a concentration of the test solution of 2500 mg/L showed a mortality of at least 50%.

II.9 Activity against Mediterranean fruitfly (Ceratitis capitata)

The active compounds were formulated in 1:3 Dimethylsulfoxide/water. 50 to 80 eggs were placed into microtiterplates filled with 0.5% agar-agar and 14% diet in water. The eggs were sprayed with 5 μl of the test solution, the plates were sealed with pierced foils and kept at 27-29° C. and 75-85% humidity under fluorescent light for 6 days. Mortality was assessed on the basis of the agility of the hatched larvae. Tests were replicated 2 times.

In this test the eggs which have been treated with 2500 ppm of the active compound 18 showed a mortality of at least 50%.

1-33. (canceled)

34. A method for protecting seed and/or the plants which grow therefrom, or for controlling animal pests comprising treating the pests, their food supply, their habitat or their breeding ground or a plant, seed, soil, area, material or environment in which the pests are growing or may grow, or the materials, plants, seeds, soils, surfaces or spaces to be protected from pest attack or infestation with a pesticidally effective amount of a compound of the formula I or a salt thereof: 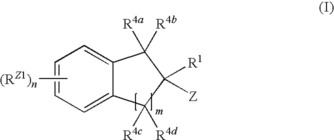
wherein
Z is a group of the formulae (II.A), (II.B) or (II.C) 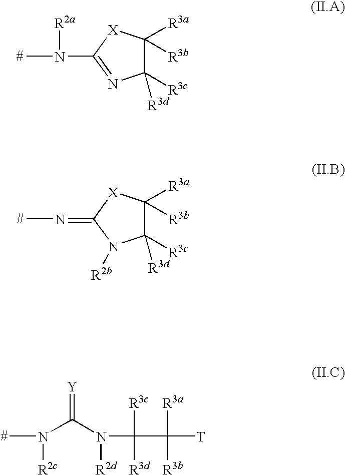
n is 0, 1, 2, 3 or 4;
m is 1 or 2;
# is the binding site to the remainder of the molecule;
X is S, O or NR^{S};
Y is S, O or NR^{5a};
T is —OR^{T1}, —SR^{T2}, —O—C(O)—R^{T3}, —O—C(S)—R^{T4} or —S—C(S)—R^{T5};
R^{T1}, R^{T2}, R^{T3}, R^{T4} and R^{T5} are selected from the group consisting of hydrogen,
C₁-C₆-alkyl, C₂-C₆-alkenyl, C₂-C₆-alkynyl, where the three last-mentioned radicals may carry 1, 2 or 3 substituents R^{Ta};
C₃-C₆-cycloalkyl which may carry 1, 2 or 3 substituents R^{Tb}; and
phenyl which may carry 1, 2 or 3 substituents R^{Tc}; where the phenyl ring may also be fused to another phenyl ring or to a 5- or 6-membered saturated, partially unsaturated or aromatic 5- or 6-membered heterocyclic ring containing 1, 2 or 3 heteroatoms selected from the group consisting of O, S and N and optionally 1 or 2 carbonyl groups as ring members, where the fused ring system may carry 1, 2 or 3 substituents R^{Tc};
saturated, partially unsaturated or aromatic 5- or 6-membered heterocyclyl containing 1, 2 or 3 heteroatoms selected from the group consisting of O, S and N and optionally 1 or 2 carbonyl groups as ring members, which may carry 1, 2 or 3 substituents R^{Td}; or
R^{T3}, R^{T4} and R^{T5} are NR^{T31}R^{T32}, where R^{T31} and R^{T32}, independently of each other, are selected from the group consisting of hydrogen, C₁-C₆-alkyl, C₂-C₆-alkenyl, C₂-C₆-alkynyl, where the 3 last-mentioned groups may carry 1, 2 or 3 substituents R^{Ta}, C₃-C₆-cycloalkyl, which may carry 1, 2 or 3 substituents R^{Tb}; aryl and aryl-C₁-C₄-alkyl, where the aryl moiety in the 2 last-mentioned groups may carry 1, 2 or 3 substituents R^{Tc}; or
R^{T31} and R^{T32} together with the nitrogen atom to which they are bound form a 3-, 4-, 5- or 6-membered saturated, partially unsaturated or aromatic 5- or 6-membered heterocyclic containing 1, 2 or 3 heteroatoms selected from the group consisting of O, S and N and optionally 1 or 2 carbonyl groups as ring members, where the heterocyclic ring may carry 1, 2 or 3 substituents R^{Td};
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl, benzyl, phenoxycarbonyl, 5- or 6-membered hetaryl and 5- or 6-membered hetarylmethyl wherein each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzoyl, phenoxycarbonyl, benzyl, benzylcarbonyl, benzyloxycarbonyl, a 5- or 6-membered saturated, partially unsaturated or aromatic heterocyclic ring, 5- or 6-membered hetarylmethyl, 5- or 6-membered hetarylcarbonyl, and 5- or 6-membered hetarylmethylcarbonyl, wherein each of the 10 last mentioned radicals may be unsubstituted or may carry any combination of 1, 2, 3, 4 or 5 radicals R^{b2}, and wherein the 5- or 6-membered heteroaromatic ring in hetarylmethyl, hetarylcarbonyl and hetarylmethylcarbonyl contains 1, 2, 3 or 4 heteroatoms selected from the group consisting of oxygen, sulfur and nitrogen as ring members, and wherein the 5- or 6-membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and where one or two methylene groups of the C₃-C₅-alkandiyl radical may be replaced by 1 or 2 non-adjacent heteroatoms or heteroatom-containing groups selected from the group consisting of O, S and NR²²;
R¹ together with R^{2b} may also be a bridging carbonyl group C(O);
R^{2c} and R^{2d}, independently of each other, are selected from the group consisting of hydrogen, formyl, C₁-C₆-alkyl, C₂-C₆-alkenyl, C₂-C₆-alkynyl, aryl, aryl-C₁-C₄-alkyl, hetaryl, hetaryl-C₁-C₄-alkyl, C₁-C₆-alkylcarbonyl, C₁-C₆-alkylthiocarbonyl, C₂-C₆-alkenylcarbonyl, C₂-C₆-alkynylcarbonyl, C₁-C₆-alkoxycarbonyl, C₁-C₆-alkoxythiocarbonyl, arylcarbonyl, hetarylcarbonyl, aryloxycarbonyl and hetaryloxycarbonyl, where the aliphatic, aromatic or heteroaromatic moieties in the 17 last-mentioned radicals may carry 1, 2, 3, 4 or 5 radicals R^{c2}, and where hetaryl is a 5- or 6-membered heteroaromatic ring containing 1, 2, 3 or 4 heteroatoms selected from the group consisting of O, S and N as ring members;
R^{3a}, R^{3b}, R^{3c}, R^{3d}, independently of each other, are selected from the group consisting of hydrogen, halogen, cyano, nitro, hydroxy, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, wherein each of the last three mentioned radicals may be unsubstituted or may carry any combination of 1, 2, 3, 4 or 5 radicals R^{b3};
R^{4a}, R^{4b}, independently of each other, are selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, wherein each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O
R^{4a} together with R^{4b} may also be C₂-Cₛ-alkandiyl which may carry 1, 2, 3, 4, 5 or 6 substituents R⁴¹ and where one or two methylene groups of the C₂-C₅-alkandiyl radical may be replaced by 1 or 2 non-adjacent heteroatoms or heteroatom-containing groups selected from the group consisting of O, S and NR⁴²; or
R^{2a} together with R^{4a} may form a bridging bivalent radical selected from the group consisting of C(O)—C(R^{24d})(R^{24b}), C(S)—C(R^{24d})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, and S(O)—NH;
R^{4c}, R^{4d} are independently defined like R^{4a} and R^{4b};
R⁵, R^{5a}, independently of each other, are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl, wherein each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
each R^{Z1} is independently selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2, 3, 4 or 5 radicals R^{az},
C(O)NR^{a}R^{b}, (SO₂)NR^{a}R^{b}, and radicals of the formula L-Cy, wherein
L is a single bond, oxygen, sulfur or C₁-C₆-alkandiyl, wherein one carbon might be replaced by oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{Z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, and C₂-C₆-alkynyl, wherein the carbon atoms in these groups may carry any combination of 1, 2 or 3 radicals R^{aw};
R^{c} has one of the meanings given for R^{a} and R^{b} or is selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino;
R^{a2}, R^{a3}, R^{a4}, R^{a5}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{bc}, R^{bz} and R^{c2} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a}, R^{24b} and R⁴¹ have independently one of the meanings given for R^{b1} or two radicals R²¹ bound to the same carbon atom may together with this carbon atom form a carbonyl group or two radicals R⁴¹ bound to the same carbon atom may together with this carbon atom form a carbonyl group;
R²², R⁴² independently are hydrogen, C₁-C₆-alkyl, C₂-C₆-alkenyl, C₁-C₆-alkynyl, where the 3 last-mentioned groups may carry 1, 2 or 3 substituents R^{a42}, C₃-C₆-cycloalkyl, which may carry 1, 2 or 3 substituents R^{b42}; aryl and aryl-C₁-C₄-alkyl, where aryl moiety in the 2 last-mentioned groups may carry 1, 2 or 3 substituents R^{c42};
each R^{Ta} is independently selected from the group consisting of halogen, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-C₄-haloalkyl, C₁-C₄-alkylcarbonyloxy and C₁-C₄-haloalkylcarbonyloxy;
each R^{Tb} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₃-C₆-cycloalkoxy, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-C₄-haloalkyl, C₁-C₄-alkylcarbonyloxy and C₁-C₄-haloalkylcarbonyloxy;
each R^{Tc} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-C₄-haloalkyl, C₁-C₄-alkylcarbonyloxy and C₁-C₄-haloalkylcarbonyloxy;
each R^{Td} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-C₄-haloalkyl, C₁-C₄-alkylcarbonyloxy and C₁-C₄-haloalkylcarbonyloxy;
R^{a42} is independently defined like R^{Ta};
R^{b42} is independently defined like R^{Tb}; and
R^{c42} is independently defined like R^{Tc}.

35. The method of claim 34, where R^{Z1} is selected from the group consisting of halogen, OH, SH, C₁-C₆-alkyl, C₁-C₆-haloalkyl, C₁-C₆-alkylthio, C₁-C₆-haloalkylthio, C₁-C₆-alkoxy and C₁-C₆-haloalkoxy.

36. The method of claim 34, where n is 0 or 1.

37. The method of claim 34, where n is 2.

38. The method of claim 34, where R¹ is hydrogen.

39. The method of claim 34, where R¹ is C₁-C₆-alkyl, phenyl or benzyl, where the phenyl moiety in the 2 last-mentioned radicals may carry 1, 2, 3, 4 or 5 radicals R^{b1}.

40. The method of claim 34, where R^{4a}, R^{4b}, R^{4c} and R^{4d} are independently selected from the group consisting of hydrogen, C₁-C₆-alkyl, C₁-C₆-haloalkyl, phenyl and benzyl, where the phenyl moiety in the 2 last-mentioned radicals may carry 1, 2, 3, 4 or 5 radicals R^{b4}.

41. The method of claim 34, where Z is a group of the formula (II.A) or (II.B).

42. The method of claim 34, where R^{2a} and R^{2b} are independently selected from the group consisting of hydrogen, C₁-C₆-alkyl, formyl, CN, C₁-C₆-alkylcarbonyl, C₁-C₆-haloalkylcarbonyl, C₁-C₆-alkoxycarbonyl, C₁-C₄-alkoxy-C₁-C₄-alkoxycarbonyl, C₁-C₆-alkylthiocarbonyl, phenyl, benzoyl, benzyl, benzylcarbonyl, a 5- or 6-membered saturated, partially unsaturated or aromatic heterocyclic ring containing 1, 2 or 3 heteroatoms selected from the group consisting of O, S and N as ring members, hetarylmethyl, hetarylcarbonyl and hetarylmethylcarbonyl, wherein each of the 8 last mentioned radicals may be unsubstituted or may carry any combination of 1, 2 or 3 radicals R^{b2}, and wherein the heteroaromatic ring in hetarylmethyl, hetarylcarbonyl and hetarylmethylcarbonyl is 5- or 6-membered and contains 1, 2, 3 or 4 heteroatoms selected from the group consisting of oxygen, sulfur and nitrogen as ring members.

43. The method of claim 42, where R^{2b} is selected from the group consisting of 5- or 6-membered saturated, partially unsaturated or aromatic heterocyclic rings containing 1, 2 or 3 heteroatoms selected from the group consisting of O, S and N as ring members, where the heterocyclic ring may carry 1, 2 or 3 radicals R^{b2}.

44. The method of claim 43, where R^{2b} is selected from the group consisting of pyrazolinyl, imidazolinyl, oxazolinyl and thiazolinyl.

45. The method of claim 34, where R^{3a}, R^{3b}, R^{3c} and R^{3d} are hydrogen.

46. The method of claim 45, where X is O.

47. The method of claim 45, where X is S.

48. The method of claim 45, where X is NR⁵.

49. The method of claim 40, where Z is a group of the formula (II.C).

50. The method of claim 49, where Y is S.

51. The method of claim 50, where R^{2c} and R^{2d} are hydrogen.

52. The method of claim 51, where T is —OR^{T1} or —O—C(O)—R^{T3}.

53. The method of claim 52, where R^{T1} is hydrogen.

54. The method of claim 52, where R^{T3} is selected from the group consisting of C₁-C₆-alkyl which may carry 1, 2 or 3 substituents R^{Ta}, phenyl which may carry 1, 2 or 3 substituents R^{Tc} and 5- or 6-membered hetaryl containing 1, 2 or 3 heteroatoms selected from the group consisting of O, S and N as ring members which may carry 1, 2 or 3 substituents R^{Td}.

55. The method of claim 34, wherein said animal pests are arthropod pests and/or nematodes.

56. The method of claim 34, wherein said animal pests are insects.

57. The method of claim 56, wherein said insects are of the order Homoptera.

58. The method of claim 56, wherein said insects are of the order Thysanoptera.

59. The method of claim 55, wherein said pests are acaridae.

60. A seed, comprising a compound of the formula I or a salt thereof: 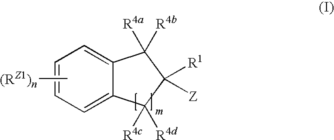
wherein
Z is a group of the formulae (II.A), (II.B) or (II.C) 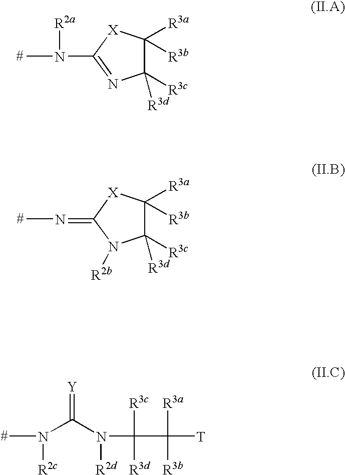
n is 0, 1, 2, 3 or 4;
m is 1 or 2;
# is the binding site to the remainder of the molecule;
X is S, O or NR⁵;
Y is S, O or NR^{5a};
T is —OR^{T1}, —SR^{T2}, —O—C(O)—R^{T3}, —O—C(S)—R^{T4} or —S—C(S)—R^{T5};
R^{T1}, R^{T2}, R^{T3}, R^{T4} and R^{T5} are selected from the group consisting of hydrogen,
C₁-C₆-alkyl, C₂-C₆-alkenyl, C₂-C₆-alkynyl, where the three last-mentioned radicals may carry 1, 2 or 3 substituents R^{Ta};
C₃-C₆-cycloalkyl which may carry 1, 2 or 3 substituents R^{Tb}; and
phenyl which may carry 1, 2 or 3 substituents R^{Tc}; where the phenyl ring may also be fused to another phenyl ring or to a 5- or 6-membered saturated, partially unsaturated or aromatic 5- or 6-membered heterocyclic ring containing 1, 2 or 3 heteroatoms selected from the group consisting of O, S and N and optionally 1 or 2 carbonyl groups as ring members, where the fused ring system may carry 1, 2 or 3 substituents R^{Tc}; and
saturated, partially unsaturated or aromatic 5- or 6-membered heterocyclyl containing 1, 2 or 3 heteroatoms selected from the group consisting of O, S and N and optionally 1 or 2 carbonyl groups as ring members, which may carry 1, 2 or 3 substituents R^{Td}; or
R^{T3}, R^{T4} and R^{T5} are NR^{T31}R^{T32}, where R^{T31} and R^{T32}, independently of each other, are selected from the group consisting of hydrogen, C₁-C₆-alkyl, C₂-C₆-alkenyl, C₂-C₆-alkynyl, where the 3 last-mentioned groups may carry 1, 2 or 3 substituents R^{Ta}, C₃-C₆-cycloalkyl, which may carry 1, 2 or 3 substituents R^{Tb}; aryl and aryl-C₁-C₄-alkyl, where the aryl moiety in the 2 last-mentioned groups may carry 1, 2 or 3 substituents R^{Tc}; or
R^{T31} and R^{T32} together with the nitrogen atom to which they are bound form a 3-, 4-, 5- or 6-membered saturated, partially unsaturated or aromatic 5- or 6-membered heterocyclic containing 1, 2 or 3 heteroatoms selected from the group consisting of O, S and N and optionally 1 or 2 carbonyl groups as ring members, where the heterocyclic ring may carry 1, 2 or 3 substituents R^{Td};
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl, benzyl, phenoxycarbonyl, 5- or 6-membered hetaryl and 5- or 6-membered hetarylmethyl, wherein each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzoyl, phenoxycarbonyl, benzyl, benzylcarbonyl, benzyloxycarbonyl, a 5- or 6-membered saturated, partially unsaturated or aromatic heterocyclic ring, 5- or 6-membered hetarylmethyl, 5- or 6-membered hetarylcarbonyl, 5- or 6-membered hetarylmethylcarbonyl, wherein each of the 10 last mentioned radicals may be unsubstituted or may carry any combination of 1, 2, 3, 4 or 5 radicals R^{b2}, and wherein the 5- or 6-membered heteroaromatic ring in hetarylmethyl, hetarylcarbonyl and hetarylmethylcarbonyl contains 1, 2, 3 or 4 heteroatoms selected from oxygen, sulfur and nitrogen as ring members, and wherein the 5- or 6-membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and where one or two methylene groups of the C₃-C₅-alkandiyl radical may be replaced by 1 or 2 non-adjacent heteroatoms or heteroatom-containing groups selected from the group consisting of O, S and NR²²;
R¹ together with R^{2b} may also be a bridging carbonyl group C(O);
R^{2c} and R^{2d}, independently of each other, are selected from the group consisting of hydrogen, formyl, C₁-C₆-alkyl, C₂-C₆-alkenyl, C₂-C₆-alkynyl, aryl, aryl-C₁-C₄-alkyl, hetaryl, hetaryl-C₁-C₄-alkyl, C₁-C₆-alkylcarbonyl, C₁-C₆-alkylthiocarbonyl, C₂-C₆-alkenylcarbonyl, C₂-C₆-alkynylcarbonyl, C₁-C₆-alkoxycarbonyl, C₁-C₆-alkoxythiocarbonyl, arylcarbonyl, hetarylcarbonyl, aryloxycarbonyl and hetaryloxycarbonyl, where the aliphatic, aromatic or heteroaromatic moieties in the 17 last-mentioned radicals may carry 1, 2, 3, 4 or 5 radicals R^{c2}, and where hetaryl is a 5- or 6-membered heteroaromatic ring containing 1, 2, 3 or 4 heteroatoms selected from the group consisting of O, S and N as ring members;
R^{3a}, R^{3b}, R^{3c}, R^{3d}, independently of each other, are selected from the group consisting of hydrogen, halogen, cyano, nitro, hydroxy, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, wherein each of the last three mentioned radicals may be unsubstituted or may carry any combination of 1, 2, 3, 4 or 5 radicals R^{b3};
R^{4a}, R^{4b}, independently of each other, are selected from the group consisting of hydrogen, halogen, C₁-C₆-alkyl, C₂-C₆-alkenyl, C₂-C₆-alkynyl, wherein the carbon atoms in these groups may carry any combination of 1, 2 or 3 radicals R^{a4},
phenyl, benzyl, 5 or 6 membered hetaryl and 5 or 6 membered hetarylmethyl, wherein each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e}; or
R^{4a} together with R^{4b} may also be C₂-C₅-alkandiyl which may carry 1, 2, 3, 4, 5 or 6 substituents R⁴¹ and where one or two methylene groups of the C₂-C₅-alkandiyl radical may be replaced by 1 or 2 non-adjacent heteroatoms or heteroatom-containing groups selected from the group consisting of O, S and NR⁴²; or
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, and S(O)—NH;
R^{4c}, R^{4d} are independently defined like R^{4a} and R^{4b};
R⁵, R^{5a}, independently of each other, are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl, wherein each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
each R^{Z1} is independently selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2, 3, 4 or 5 radicals R^{az},
C(O)NR^{a}R^{b}, (SO₂)NR^{a}R^{b}, and radicals of the formula L-Cy, wherein
L is a single bond, oxygen, sulfur or C₁-C₆-alkandiyl, wherein one carbon might be replaced by oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{Z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, and C₂-C₆-alkynyl, wherein the carbon atoms in these groups may carry any combination of 1, 2 or 3 radicals R^{aw};
R^{c} has one of the meanings given for R^{a} and R^{b} or is selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino;
R^{a2}, R^{a3}, R^{a4}, R^{a5}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{bc}, R^{bz} and R^{c2} are independently of each other selected from the group consisting of halogen, cyano, nitro, hydroxy, mercapto, amino, carboxyl, C₁-C₆-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a}, R^{24b} and R⁴¹ have independently one of the meanings given for R^{b1} or two radicals R²¹ bound to the same carbon atom may together with this carbon atom form a carbonyl group or two radicals R⁴¹ bound to the same carbon atom may together with this carbon atom form a carbonyl group;
R²², R⁴² independently are hydrogen, C₁-C₆-alkyl, C₂-C₆-alkenyl, C₁-C₆-alkynyl, where the 3 last-mentioned groups may carry 1, 2 or 3 substituents R^{a42}, C₃-C₆-cycloalkyl, which may carry 1, 2 or 3 substituents R^{b42}; aryl and aryl-C₁-C₄-alkyl, where aryl moiety in the 2 last-mentioned groups may carry 1, 2 or 3 substituents R^{c42};
each R^{Ta} is independently selected from the group consisting of halogen, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-C₄-haloalkyl, C₁-C₄-alkylcarbonyloxy and C₁-Cₐ-haloalkylcarbonyloxy;
each R^{Tb} is independently selected from the group consisting of halogen, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-Cₐ-haloalkylthio, C₃-C₆-cycloalkoxy, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-Cₐ-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-Cₐ-alkyl, C₁-Cₐ-haloalkyl, C₁-C₄-alkylcarbonyloxy and C₁-C₄-haloalkylcarbonyloxy;
each R^{Tc} is independently selected from the group consisting of halogen, C₁-Cₐ-alkyl, C₁-C₄-haloalkyl, C₃-C₆-cycloalkyl, C₃-C₆-halocycloalkyl, C₁-Cₐ-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-C₄-haloalkyl, C₁-C₄-alkylcarbonyloxy and C₁-C₄-haloalkylcarbonyloxy;
each R^{Td} is independently selected from the group consisting of halogen, C₁-Cₐ-alkyl, C₁-Cₐ-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-Cₐ-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-Cₐ-alkyl, C₁-C₄-haloalkyl, C₁-C₄-alkylcarbonyloxy and C₁-C₄-haloalkylcarbonyloxy;
R^{a42} is independently defined like R^{Ta};
R^{b42} is independently defined like R^{Tb}; and
R^{c42} is independently defined like R^{Tc}.

61. A method for treating or protecting an animal from infestation or infection by parasites which comprises bringing the animal in contact with a parasiticidally effective amount of a compound of the formula I or a salt thereof: 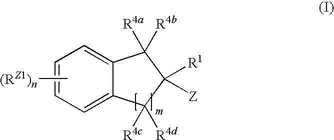
wherein
Z is a group of the formulae (II.A), (II.B) or (II.C) 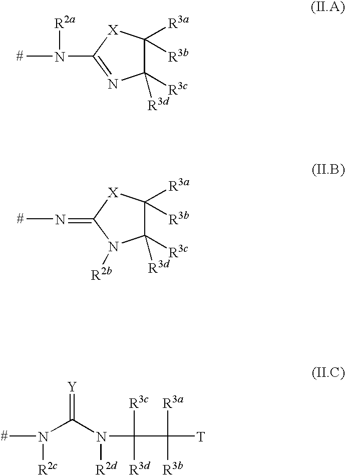
n is 0, 1, 2, 3 or 4;
m is 1 or 2;
# is the binding site to the remainder of the molecule;
X is S, O or NR⁵;
Y is S, O or NR^{5a};
T is —OR^{T1}, —SR^{T2}, —O—C(O)—R^{T3}, —O—C(S)—R^{T4} or —S—C(S)—R^{T5};
R^{T1}, R^{T2}, R^{T3}, R^{T4} and R^{T5} are selected from hydrogen,
C₁-C₆-alkyl, C₂-C₆-alkenyl, C₂-C₆-alkynyl, where the three last-mentioned radicals may carry 1, 2 or 3 substituents R^{Ta};
C₃-C₆-cycloalkyl which may carry 1, 2 or 3 substituents R^{Tb}; and
phenyl which may carry 1, 2 or 3 substituents R^{Tc}; where the phenyl ring may also be fused to another phenyl ring or to a 5- or 6-membered saturated, partially unsaturated or aromatic 5- or 6-membered heterocyclic ring containing 1, 2 or 3 heteroatoms selected from the group consisting of O, S and N and optionally 1 or 2 carbonyl groups as ring members, where the fused ring system may carry 1, 2 or 3 substituents R^{Tc};
saturated, partially unsaturated or aromatic 5- or 6-membered heterocyclyl containing 1, 2 or 3 heteroatoms selected from the group consisting of O, S and N and optionally 1 or 2 carbonyl groups as ring members, which may carry 1, 2 or 3 substituents R^{Td}; or
R^{T3}, R^{T4} and R^{T5} are NR^{T31}R^{T32}, where R^{T31} and R^{T32}, independently of each other, are selected from the group consisting of hydrogen, C₁-C₆-alkyl, C₂-C₆-alkenyl, C₂-C₆-alkynyl, where the 3 last-mentioned groups may carry 1, 2 or 3 substituents R^{Ta}, C₃-C₆-cycloalkyl, which may carry 1, 2 or 3 substituents R^{Tb}; aryl and arylC₁-C₄-alkyl, where the aryl moiety in the 2 last-mentioned groups may carry 1, 2 or 3 substituents R^{Tc}; or
R^{T31} and R^{T32} together with the nitrogen atom to which they are bound form a 3-, 4-, 5- or 6-membered saturated, partially unsaturated or aromatic 5- or 6-membered heterocyclic containing 1, 2 or 3 heteroatoms selected from the group consisting of O, S and N and optionally 1 or 2 carbonyl groups as ring members, where the heterocyclic ring may carry 1, 2 or 3 substituents R^{Td};
R¹ is selected from the group consisting of hydrogen, cyano, C₁-C₆-alkyl, C₁-C₆-haloalkyl, C₂-C₆-alkenyl, C₂-C₆-alkynyl, C₁-C₆-alkoxycarbonyl, C₁-C₆-alkylcarbonyl, C₂-C₆-alkenylcarbonyl, C₂-C₆-alkynylcarbonyl;
C₃-C₆-cycloalkyl, phenyl, benzyl, phenoxycarbonyl, 5- or 6-membered hetaryl and 5- or 6-membered hetarylmethyl, wherein each of the six last mentioned radicals may be unsubstituted or may carry any combination of 1, 2, 3, 4 or 5 radicals R^{b1};
R^{2a}, R^{2b} are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, (C₁-C₆-alkyl)thiocarbonyl, (C₁-C₆-alkoxy)thiocarbonyl, wherein the carbon atoms in the aliphatic radicals of the aforementioned groups may carry any combination of 1, 2 or 3 radicals R^{a2},
C(O)NR^{a}R^{b}, C(S)NR^{a}R^{b}, (SO₂)NR^{a}R^{b},
phenyl, benzoyl, phenoxycarbonyl, benzyl, benzylcarbonyl, benzyloxycarbonyl, a 5- or 6-membered saturated, partially unsaturated or aromatic heterocyclic ring, 5- or 6-membered hetarylmethyl, 5- or 6-membered hetarylcarbonyl, and 5- or 6-membered hetarylmethylcarbonyl, wherein each of the 10 last mentioned radicals may be unsubstituted or may carry any combination of 1, 2, 3, 4 or 5 radicals R^{b2}, and wherein the 5- or 6-membered heteroaromatic ring in hetarylmethyl, hetarylcarbonyl and hetarylmethylcarbonyl contains 1, 2, 3 or 4 heteroatoms selected from the group consisting of oxygen, sulfur and nitrogen as ring members, and wherein the 5- or 6-membered heterocyclic ring contains 1, 2 or 3 heteroatoms selected from the group consisting of oxygen, sulfur and nitrogen as ring members; or
R¹ together with R^{2a} may be C₃-C₅-alkandiyl which may carry 1, 2, 3, 4 or 5 radicals R²¹ and where one or two methylene groups of the C₃-C₅-alkandiyl radical may be replaced by 1 or 2 non-adjacent heteroatoms or heteroatom-containing groups selected from the group consisting of O, S and NR²²;
R¹ together with R^{2b} may also be a bridging carbonyl group C(O);
R^{2c} and R^{2d}, independently of each other, are selected from the group consisting of hydrogen, formyl, C₁-C₆-alkyl, C₂-C₆-alkenyl, C₂-C₆-alkynyl, aryl, aryl-C₁-C₄-alkyl, hetaryl, hetaryl-C₁-C₄-alkyl, C₁-C₆-alkylcarbonyl, C₁-C₆-alkylthiocarbonyl, C₂-C₆-alkenylcarbonyl, C₂-C₆-alkynylcarbonyl, C₁-C₆-alkoxycarbonyl, C₁-C₆-alkoxythiocarbonyl, arylcarbonyl, hetarylcarbonyl, aryloxycarbonyl and hetaryloxycarbonyl, where the aliphatic, aromatic or heteroaromatic moieties in the 17 last-mentioned radicals may carry 1, 2, 3, 4 or 5 radicals R^{c2}, and where hetaryl is a 5- or 6-membered heteroaromatic ring containing 1, 2, 3 or 4 heteroatoms selected from O, S and N as ring members;
R^{3a}, R^{3b}, R^{3c}, R^{3d} independently of each other, are selected from the group consisting of hydrogen, halogen, cyano, nitro, hydroxy, mercapto, amino, C₁-C₆-haloalkyl, C₁-C₆-alkyl, C₁-C₆-alkylamino, di-(C₁-C₆-alkyl)amino, C₁-C₆-alkoxy, wherein the carbon atoms in the last 4 mentioned radicals may be unsubstituted or may carry any combination of 1, 2 or 3 radicals R^{a3},
C₃-C₆-cycloalkyl, phenyl and benzyl, wherein each of the last three mentioned radicals may be unsubstituted or may carry any combination of 1, 2, 3, 4 or 5 radicals R^{b3};
R^{4a}, R^{4b}, independently of each other, are selected from the group consisting of hydrogen, halogen, C₁-C₆-alkyl, C₂-C₆-alkenyl, C₂-C₆-alkynyl, C₁-C₆-haloalkyl, wherein the carbon atoms in these groups may carry any combination of 1, 2 or 3 radicals R^{a4},
phenyl, benzyl, 5 or 6 membered hetaryl and 5 or 6 membered hetarylmethyl, wherein each of the four last mentioned radicals may be unsubstituted or may carry any combination of 1, 2, 3, 4 or 5 radicals R^{b4}, and wherein the 5 or 6 membered heteroaromatic ring in hetarylmethyl and hetaryl contains 1, 2, 3 or 4 heteroatoms selected from the group consisting of oxygen, sulfur and nitrogen as ring members; or
R^{4a} together with R^{4b} may also be ═O, ═NR^{c} or ═CR^{d}R^{e}; or
R^{4a} together with R^{4b} may also be C₂-C₅-alkandiyl which may carry 1, 2, 3, 4, 5 or 6 substituents R⁴¹ and where one or two methylene groups of the C₂-C₅-alkandiyl radical may be replaced by 1 or 2 non-adjacent heteroatoms or heteroatom-containing groups selected from the group consisting of O, S and NR⁴²; or
R^{2a} together with R^{4a} may form a bridging bivalent radical selected from the group consisting of C(O)—C(R^{24a})(R^{24b}), C(S)—C(R^{24a})(R^{24b}), CH₂—C(R^{24a})(R^{24b}), S(O)₂—C(R^{24a})(R^{24b}), S(O)—C(R^{24a})(R^{24b}), C(O)—O, C(S)—O, S(O)₂—O, S(O)—O, C(O)—NH, C(S)—NH, S(O)₂—NH, and S(O)—NH;
R^{4c}, R^{4d} are independently defined like R^{4a} and R^{4b};
R⁵, R^{5a}, independently of each other, are selected from the group consisting of hydrogen, formyl, CN, C₁-C₆-alkyl, C₂-C₆-alkenyl, C₂-C₆-alkynyl, C₁-C₆-alkylcarbonyl, C₂-C₆-alkenylcarbonyl, C₂-C₆-alkynylcarbonyl, C₁-C₆-alkoxycarbonyl, C₁-C₆-alkylthiocarbonyl, wherein the carbon atoms in the aliphatic radicals of the aforementioned groups may carry any combination of 1, 2 or 3 radicals R^{a5},
C(O)NR^{a}R^{b}, (SO₂)NR^{a}R^{b}, C(S)NR^{a}R^{b}
phenyl, benzyl, phenoxycarbonyl, 5 or 6 membered hetarylmethyl, 5 or 6 membered hetarylcarbonyl and benzoyl, wherein each of the last six mentioned radicals may be unsubstituted or may carry any combination of 1, 2, 3, 4 or 5 radicals R^{b5}, and wherein the 5 or 6 membered heteroaromatic ring in hetarylmethyl and hetarylcarbonyl contains 1, 2, 3 or 4 heteroatoms selected from the group consisting of oxygen, sulfur and nitrogen as ring members;
each R^{Z1} is independently selected from the group consisting of halogen, OH, SH, SO₃H, COOH, cyano, nitro, C₁-C₆-alkyl, C₁-C₆-alkoxy, C₁-C₆-alkylthio, C₂-C₆-alkenyl, C₂-C₆-alkenyloxy, C₂-C₆-alkenylthio, C₂-C₆-alkynyl, C₂-C₆-alkynyloxy, C₂-C₆-alkynylthio, C₁-C₆-alkylsulfonyl, C₁-C₆-alkylsulfoxyl, C₂-C₆-alkenylsulfonyl, C₂-C₆-alkynylsulfonyl, a radical NR^{a}R^{b}, formyl, C₁-C₆-alkylcarbonyl, C₂-C₆-alkenylcarbonyl, C₂-C₆-alkynylcarbonyl, C₁-C₆-alkoxycarbonyl, C₂-C₆-alkenyloxycarbonyl, C₂-C₆-alkynyloxycarbonyl, formyloxy, C₁-C₆-alkylcarbonyloxy, C₂-C₆-alkenylcarbonyloxy, C₂-C₆-alkynylcarbonyloxy, wherein the carbon atoms in the aliphatic radicals of the aforementioned groups may carry any combination of 1, 2, 3, 4 or 5 radicals R^{az},
C(O)NR^{a}R^{b}, (SO₂)NR^{a}R^{b}, and radicals of the formula L-Cy, wherein
L is a single bond, oxygen, sulfur or C₁-C₆-alkandiyl, wherein one carbon might be replaced by oxygen,
Cy is selected from the group consisting of C₃-C₁₂-cycloalkyl, which is unsubstituted or substituted with any combination of 1, 2, 3, 4 or 5 radicals R^{bz}, phenyl, naphthyl and mono- or bicyclic 5- to 10-membered heterocyclyl, which contains 1, 2, 3 or 4 heteroatoms selected from the group consisting of oxygen, sulfur and nitrogen as ring members, wherein Cy is unsubstituted or may carry any combination of 1, 2, 3, 4 or 5 radicals R^{bz};
and wherein two radicals R^{Z1} that are bound to adjacent carbon atoms may form together with said carbon atoms a fused benzene ring, a fused saturated or partially unsaturated 5, 6, or 7 membered carbocycle or a fused 5, 6, or 7 membered heterocycle, which contains 1, 2, 3 or 4 heteroatoms selected from the group consisting of oxygen, sulfur and nitrogen as ring members, and wherein the fused ring is unsubstituted or may carry any combination of 1, 2, 3, or 4 radicals R^{bz};
R^{a}, R^{b} are independently of each other selected from the group consisting of hydrogen, C₁-C₆-alkyl, phenyl, benzyl, 5 or 6 membered hetaryl, C₂-C₆-alkenyl, and C₂-C₆-alkynyl, wherein the carbon atoms in these groups may carry any combination of 1, 2 or 3 radicals R^{aw};
R_{c} has one of the meanings given for R^{a} and R^{b} or is selected from the group consisting of C₁-C₆-alkoxy, OH, NH₂, C₁-C₆-alkylamino, di(C₁-C₆-alkyl)amino, arylamino, N—(C₁-C₆-alkyl)-N-arylamino and diarylamino, wherein aryl is phenyl which may be unsubstituted or may carry 1, 2 or 3 substituents R^{bc};
R^{d}, R^{e} have one of the meanings given for R^{a} and R^{b} or are independently of each other selected from the group consisting of C₁-C₆-alkoxy and di(C₁-C₆-alkyl)amino;
R^{a2}, R^{a3}, R^{a4}, R^{a5}, R^{aw} and R^{az} are independently of each other selected from the group consisting of halogen, cyano, nitro, hydroxy, mercapto, amino, carboxyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylcarbonyl, C₁-C₆-alkoxycarbonyl, C₁-C₆-alkylthio, C₁-C₆-haloalkylthio, C₁-C₆-alkylsulfonyl and C₁-C₆-haloalkylsulfonyl;
R^{b1}, R^{b2}, R^{b3}, R^{b4}, R^{b5}, R^{bz} and R^{c2} are independently of each other selected from the group consisting of halogen, cyano, nitro, hydroxy, mercapto, amino, carboxyl, C₁-Cₒ-alkyl, C₁-C₆-haloalkyl, C₃-C₆-cycloalkyl, C₁-C₆-alkoxy, C₂-C₆-alkenyloxy, C₂-C₆-alkynyloxy, C₁-C₆-haloalkoxy, C₁-C₆-alkylthio, C₁-C₆-alkylamino, di(C₁-C₆-alkyl)amino, C₁-C₆-alkylsulfonyl, C₁-C₆-alkylsulfoxyl, formyl, C₁-C₆-alkylcarbonyl, C₁-C₆-alkoxycarbonyl, formyloxy, and C₁-C₆-alkylcarbonyloxy;
R²¹, R^{24a}, R^{24b} and R⁴¹ have independently one of the meanings given for R^{b1} or two radicals R²¹ bound to the same carbon atom may together with this carbon atom form a carbonyl group or two radicals R⁴¹ bound to the same carbon atom may together with this carbon atom form a carbonyl group;
R²², R⁴² independently are hydrogen, C₁-C₆-alkyl, C₂-C₆-alkenyl, C₁-C₆-alkynyl, where the 3 last-mentioned groups may carry 1, 2 or 3 substituents R^{a42}, C₃-C₆-cycloalkyl, which may carry 1, 2 or 3 substituents R^{b42}; aryl or aryl-C₁-C₄-alkyl, where aryl moiety in the 2 last-mentioned groups may carry 1, 2 or 3 substituents R^{c42};
each R^{Ta} is independently selected from the group consisting of halogen, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-C₄-alkyl, C₁-C₄-haloalkyl, C₁-C₄-alkylcarbonyloxy and C₁-C₄-haloalkylcarbonyloxy;
each R^{Tb} is independently selected from the group consisting of halogen, C₁-Cₐ-alkyl, C₁-C₄-haloalkyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₃-C₆-cycloalkoxy, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-C₄-alkyl, C₁-Cₐ-haloalkyl, C₁-C₄-alkylcarbonyloxy and C₁-C₄-haloalkylcarbonyloxy;
each R^{Tc} is independently selected from the group consisting of halogen, C₁-C₄-alkyl, C₁-Cₐ-haloalkyl, C₃-C₆-cycloalkyl, C₃-C₆-halocycloalkyl, C₁-C₄-alkoxy, C₁-C₄-haloalkoxy, C₁-Cₐ-alkylthio, C₁-Cₐ-haloalkylthio, C₁-C₄-alkylcarbonyl, C₁-C₄-haloalkylcarbonyl, C₁-C₄-alkoxycarbonyl, C₁-C₄-haloalkoxycarbonyl, C₁-C₄-alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from the group consisting of halogen, C₁-Cₐ-alkyl, C₁-C₄-haloalkyl, C₁-C₄-alkylcarbonyloxy and C₁-Cₐ-haloalkylcarbonyloxy;
each R^{Td} is independently selected from the group consisting of halogen, C₁-C₄-alkyl, C₁-Cₐ-haloalkyl, C₃-C₆-cycloalkyl, C₃-C₆-halocycloalkyl, C₁-C₄-alkoxy, C₁-C₄-haloalkoxy, C₁-Cₐ-alkylthio, C₁-C₄-haloalkylthio, C₁-C₄-alkylcarbonyl, C₁-C₄-haloalkylcarbonyl, C₁-C₄-alkoxycarbonyl, C₁-C₄-haloalkoxycarbonyl, alkylcarbonyloxy, C₁-C₄-haloalkylcarbonyloxy, phenyl, phenyloxy, phenylthio and 5- or 6-membered hetaryl, where the phenyl moiety and the hetaryl ring in the four last-mentioned substituents may carry 1, 2 or 3 substituents selected from halogen, C₁-C₄-alkyl, C₁-Cₐ-haloalkyl, C₁-C₄-alkylcarbonyloxy and C₁-C₄-haloalkylcarbonyloxy;
R^{a42} is independently defined like R^{Ta};
R^{b42} is independently defined like R^{Tb}; and
R^{c42} is independently defined like R^{TC}.

62. Compounds of formula (I) and salts thereof 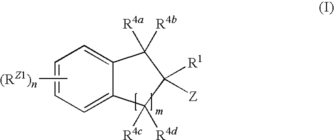
wherein
Z is a group of the formulae (II.A), (II.B) or (EC) 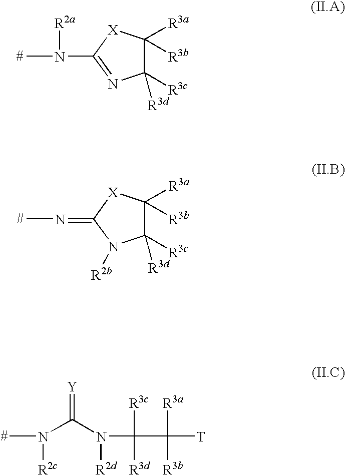
and wherein X, Y, R¹, R^{2a}, R^{2b}, R^{2c}, R^{2d}, R^{3a}, R^{3b}, R^{3c}, R^{3d}, R^{4a}, R^{4b}, R^{4c}, R^{4d}, R^{Z1}, T, m and n have the meanings given in claim 34;
except for compounds wherein n is 0, or n is 1 or 2 with R^{Z1} being C₁-C₆-alkyl, C₁-C₆-alkoxy or halogen, Z is a group of formula (II.A) or (II.B) and R^{2a}, R^{2b}, R^{4a}, R^{4b}, R^{4c} and R^{4d} are all hydrogen.

63. Compounds of formula (I) and salts thereof 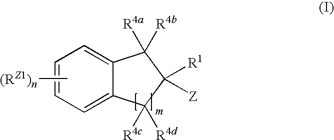
wherein
Z is a group of the formulae (II.A), (II.B) or (II.C) 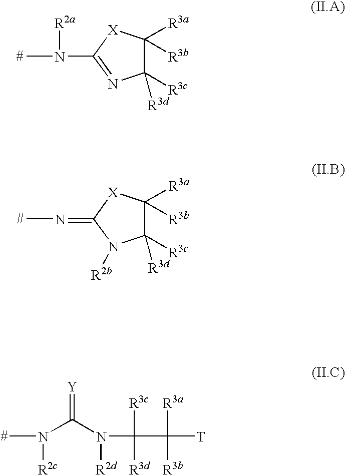
and wherein X, Y, R¹, R^{2a}, R^{2b}, R^{2c}, R^{2d}, R^{3a}, R^{3b}, R^{3c}, R^{3d}, R^{4a}, R^{4b}, R^{4c}, R^{4d}, R^{Z1}, T, m and n have the meanings given in claim 34;
except for compounds wherein n is 0 or n is 1 or 2 with R^{Z1} being C₁-C₆-alkyl, C₁-C₆-alkoxy, halogen, phenyl or naphthyl, and simultaneously Z is a group of formula (II.A) or (II.B), R^{2a}, R^{2b}, R^{3a}, R^{3b}, R^{3c} and R^{3d} are all hydrogen and one of R^{4a} and R^{4b} is hydrogen and the other is hydrogen or C₁-C₆-alkyl and one of R^{4c} and R^{4d} is hydrogen and the other is hydrogen or C₁-C₆-alkyl.
